(19) United States
(12) Patent Application Publication
(10) Pub. No.: US 2023/0064809 A1
(43) Pub. Date: Mar. 2, 2023

(54) SUBSTITUTED AMINOQUINOLONES AS DGKALPHA INHIBITORS FOR IMMUNE ACTIVATION

(71) Applicants: Bayer Aktiengesellschaft, Leverkusen (DE); Bayer Pharma Aktiengesellschaft, Berlin (DE); Deutsches Krebsforschungszentrum, Heidelberg (DE)

(72) Inventors: Norbert SCHMEES, Berlin (DE); Lars WORTMANN, Biberbach an der Riß (DE); Dennis KIRCHHOFF, Berlin (DE); Thi Thanh Uyen NGUYEN, Berlin (DE); Nicolas WERBECK, Berlin (DE); Ulf BÖMER, Glienicke (DE); Kirstin PETERSEN, Berlin (DE); Christina KOBER, Lollar (DE); Detlef STÖCKIGT, Potsdam (DE); Christian LECHNER, Berlin (DE); Rienk OFFRINGA, Heidelberg (DE)

(73) Assignees: Bayer Aktiengesellschaft, Leverkusen (DE); Bayer Pharma Aktiengesellschaft, Berlin (DE); Deutsches Krebsforschungszentrum, Heidelberg (DE)

(21) Appl. No.: 17/780,814

(22) PCT Filed: Nov. 24, 2020
(86) PCT No.: PCT/EP2020/083197

§ 371 Date: May 27, 2022

(30) Foreign Application Priority Data
Nov. 28, 2019 (EP) 19212257.0

(51) Int. Cl. A61K 31/4709; C07D 471/10; C07D 487/10; A61K 31/5377; A61K 31/496; A61P 37/04; A61P 35/00; A61K 45/06

(52) U.S. Cl. CPC A61K 31/4709; C07D 471/10; C07D 487/10; A61K 31/5377; A61K 31/496; A61P 37/04; A61P 35/00; A61K 45/06

(57) ABSTRACT

The present invention covers aminoquinolone compounds of general formula (I), in which R¹, R², R³, R⁴, R⁵, R⁶, R⁷, R⁸, m, n, o and p are as defined herein, methods of preparing said compounds, intermediate compounds useful for preparing said compounds, pharmaceutical compositions and combinations comprising said compounds and the use of said compounds for manufacturing pharmaceutical compositions for the treatment and/or prophylaxis of diseases, in particular of diacylglycerol kinase alpha regulated disorders, as a sole agent or in combination with other active ingredients.

The present invention covers substituted aminoquinolone compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of diacylglycerol kinase alpha (DGKalpha, DGKα) regulated disorders, as a sole agent or in combination with other active ingredients.

The compounds of general formula (I) inhibit DGKα and enhance T cell mediated immune response. This is a new strategy to use the patient's own immune system to overcome immunoevasive strategies utilized by many neoplastic disorders, respectively cancer and by this enhancing anti-tumor immunity. Furthermore, said compounds are used in particular to treat disorders such as viral infections or conditions with dysregulated immune responses or other disorders associated with aberrant DGKα signaling.

The present invention further relates to the use, respectively to the use of the compounds of general formula (I) for manufacturing pharmaceutical compositions for enhancement of T cell mediated immune response.

The present invention further relates to the use, respectively to the use of the compounds of general formula (I) for manufacturing pharmaceutical compositions for the treatment of cancer.

The present invention further relates to the use, respectively to the use of the compounds of general formula (I) for manufacturing pharmaceutical compositions for the treatment or prophylaxis of fibrotic disorders, virus infections, cardiac diseases and lymphoproliferative disorders.

### BACKGROUND

Diacylglycerol kinases (DGKs) represent a family of enzymes that catalyze phosphorylation of the membrane lipid sn-1,2 diacylglycerol (DAG) to form phosphatidic acid (PA) (Eichmann and Lass, Cell Mol Life Sci. 2015; 72: 3931). In T cells, DAG is formed downstream of the T cell receptor (TCR) after activation of the gamma 1 isoform of phospholipase C (PLCγ1) and cleavage of phosphatidylinositol 4,5-biphosphate (PIP2) into DAG and an additional second messenger, inositol 1,4,5-triphosphate (IP3) (Krishna and Zhong, Front. Immunol 2013, 4, 178). Whereas, IP3 is important in facilitating release of calcium from the endoplasmic reticulum, DAG interacts with other proteins important in TCR signal transduction, such as Protein kinase Cθ (Quann et al., Nat Immunol 2011(7), 647) and the Ras activating protein RasGRP1 (Krishna and Zhong, Front. Immunol 2013, 4:178). Although, three isoforms of DGK are known to be present within T cells [DGKα (DGKalpha), DGKδ (DGKdelta), and DGKζ (DGKzeta)], only two, DGKα and DGKζ, are thought to play an important role in facilitating DAG metabolism downstream of the TCR (Joshi and Koretzky, Int. J. Mol. Sci. 2013, 14, 6649).

Targeting the activity of DGKα in T cells, either by germline deletion, or with chemical inhibitors, results in enhanced and sustained signaling downstream of T cells, as assessed by prolonged phosphorylation of downstream molecules, such as extracellular signal-related kinases 1/2 (ERK1/2 (Zhong et al., Nat Immunol 2003, 4, 882; Olenchock et al., Nat Immunol 2006, 7, 1174; Riese et al., J. Biol. Chem 2011, 286, 5254). Furthermore, the overexpression of DGKα induces a state of decreased functional activity resembling an anergy-like state (Zha et al., Nat Immunol 2006, 7, 1166). In contrast, deletion of DGKα in T cells with enhanced production of effector cytokines, such as IL2 and IFNγ, and enhanced proliferation (Zhong et al., Nat Immunol 2003, 4, 882 Olenchock et al., Nat Immunol 2006, 7, 1174).

These findings suggest that DGKα might serve as a useful target for enhancing T cell anti-tumor activity. The role of DGKα in anti-tumor responses was studied recently in human tumor-infiltrating CD8+ T cells (CD8-TILs) from patients with renal cell carcinoma (RCC) (Prinz et al., J. Immunol 2012, 188, 5990). CD8-TILs from RCCs were defective in lytic granule exocytosis and their ability to kill target cells. While proximal signaling events were intact in response to TCR engagement, CD8-TILs exhibited decreased phosphorylation of ERK when compared to non-tumor-infiltrating CD8+ T cells. Treatment of CD8-TILs with an inhibitor of DGKα activity rescued killing ability of target cells, increased basal levels of phosphorylation of ERK, and increased PMA/ionomycin-stimulated phosphorylation of ERK.

In addition, Arranz-Nicolas et al show that DGK inhibitors promoted not only Ras/ERK signaling but also AP-1 (Activator protein-1) transcription, facilitated DGKα membrane localization, reduced the requirement for costimulation, and cooperated with enhanced activation following DGKζ silencing/deletion. In contrast with enhanced activation triggered by pharmacological inhibition, DGKα silencing/genetic deletion led to impaired Lck (lymphocyte-specific protein tyrosine kinase) activation and limited costimulation responses. (Arranz-Nicolas et al., Canc Immun, Immunother 2018, 67(6), 965).

In addition, antigen-specific CD8⁺ T cells from DGKα^{-/-} and DGKζ^{-/-} mice show enhanced expansion and increased cytokine production following (Lymphocytic choriomeningitis virus) infection (Shin et al. J. Immunol, 2012).

Additionally, the adoptive transfer of CAR (chimeric antigen receptor)-T cells deficient in DGKα demonstrated increased efficacy compared to wild type CAR T cells T cells in the treatment of murine mesothelioma (Riese et al., Cancer Res 2013, 73(12), 3566) and a glioblastoma xenograft mouse model (Jung et al. Cancer Res. 2018, 78(16), 4692).

Apart from T-cell regulation, DGKα also plays a role in cancer, mediating numerous aspects of cancer cell progression including survival (Bacchiocchi et al., Blood, 2005, 106(6), 2175; Yanagisawa et al. Biochim Biophys Acta 2007, 1771, 462), migration and invasion of cancer cells (Baldanzi et al., Oncogene 2008, 27, 942; Filigheddu et al., Anticancer Res 2007, 27, 1489; Rainero et al., J Cell Biol 2012, 196(2): 277). In particular, it has been reported that DGKα is over expressed in hepatocellular carcinoma (Takeishi et al., J Hepatol 2012, 57, 77) and melanoma cells (Yanagisawa et al., Biochim Biophys Acta 2007, 1771, 462) while other reports suggested that the growth of colon and breast cancer cell lines was significantly inhibited by DGKα-siRNA16 and DGKα/atypical PKC/b1 integrin signalling pathway was crucial for matrix invasion of breast carcinoma cells (Rainero et al., PLoS One 2014, 9(6): e97144) In addition, expression is also higher in lymphonodal metastasis than in breast original tumour (Hao et al., Cancer 2004, 100, 1110).

Additionally, a study testing the importance of DGKα in glioblastoma multiforme (GBM) cells found that concurrent administration of the relatively non-specific DGKα inhibitor R59022 resulted in decreased growth of intracranially injected GBM tumors. (Dominguez et al. Cancer Discov 2013, 3(7): 782).

Also, DGKα promotes esophageal squamous cell carcinoma (ESCC) progression, supporting DGKα as a potential target for ESCC therapy (Chen et al., Oncogene, 2019, 38 (14) 2533).

In addition, pharmacological inhibition of DGK diminished both airway inflammation and airway hyperresponsiveness in mice and also reduced bronchoconstriction of human airway samples in vitro by blocking T helper 2 (T_{H}2) differentiation (Singh et al., Sci Signal. 2019, 12, eaax3332).

Furthermore, inhibition of DGKα has the potential to reverse the life-threatening Epstein-Barr virus (EBV)-associated immunopathology that occurs in patients X-linked lymphoproliferative disease (XLP-1) patients (Ruffo et al., Sci Transl Med. 2016, 13, 8, 321; Velnati et al., Eur J Med Chem. 2019, 164, 378).

In addition, DGKα exacerbates cardiac injury after ischemia/reperfusioncardiac diseases (Sasaki et al., Heart Vessels, 2014, 29, 110).

Taken together, the findings from these studies argue that restraining DGKα activity in T cells and tumor cells may prove valuable in generating more vigorous immune responses against pathogens and tumors and in amoiroting Th2 driven (ato) immune diseases (in re-balancing the immune-systeme). In addition, inhibiting DGKα activity has a therapeutic potential in targeting tumors directly as well as addressing fibrotic disorders, virus infection associated pathologies, cardiac diseases and lymphoproliferative disorders.

### PRIOR ART

DGKα ignitors were reported in the literature. R59022 (A) was identified to act on DGKα in red blood cells (de Chaffoy de Courcelles et. al., J. Biol. Chem. Vol 260, No. 29, (1985), p15762-70). Structurally related R59949 (B) was identified to act on DGKα in T-lymphocytes by inhibiting the transformation of 1,2-diacylglycerols to their respective phosphatidic acids (Jones et. al., J. Biol. Chem. Vol 274, No. 24, (1999), p16846-52). Ritanserin (C), originally identified as a serotonine receptor antagonist, showed comparable activity on DGKα such as the two R cpds (A) and (B) (Boroda et. al., BioChem. Pharm. 123, (2017), 29-39).

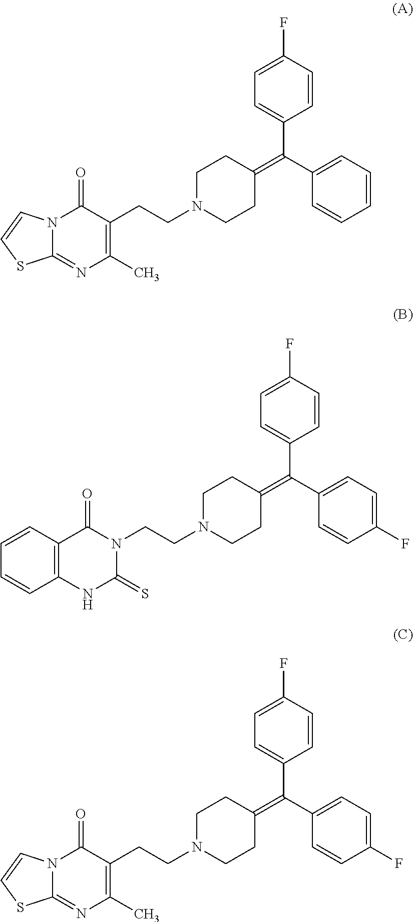 [SMILES: Cc1nc2sccn2c(=O)c1CCN1CCC(=C(c2ccccc2)c2ccc(F)cc2)CC1 ; O=c1c2ccccc2[nH]c(=S)n1CCN1CCC(=C(c2ccc(F)cc2)c2ccc(F)cc2)CC1 ; Cc1nc2sccn2c(=O)c1CCN1CCC(=C(c2ccc(F)cc2)c2ccc(F)cc2)CC1]

A further structure, CU-3 (D) was identified as a first compound with sub-micromolar inhibitory activity on DGKα (Sakane et. al., J. Lipid Res. Vol 57, (2016), p368-79).

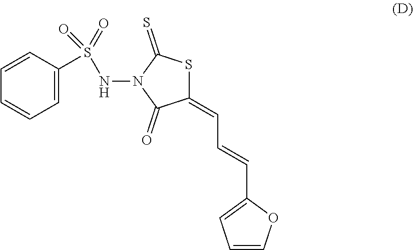 [SMILES: O=C1/C(=C\C=C\c2ccco2)SC(=S)N1NS(=O)(=O)c1ccccc1]

AMB639752 (E) was describe as a further DGKα selective inhibitor with micromolar activity (S. Velnati et al. Eur J. Med. Chem 2019, 164, p378-390).

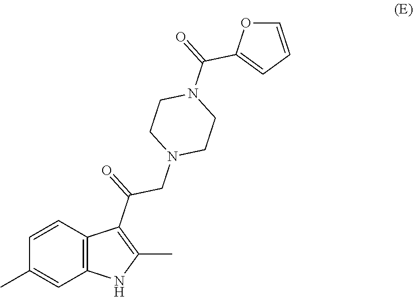 [SMILES: Cc1ccc2c(C(=O)CN3CCN(C(=O)c4ccco4)CC3)c(C)[nH]c2c1]

WO2020/151636 relates to azaquinolinones as PDE9 inhibitor compounds for treatment of PDE9 mediated diseases.

WO2020/143626 relates to quinolinones as PDE9 inhibitor compounds for treatment of PDE9 mediated diseases.

WO2019/241157 describe Naphthydrin compounds as KRAS G12C inhibitors for treatment of disorders, among them pancreatic, colorectal and lung cancers.

WO2020/006016 and WO2020/006018 describe Naphthydrinone compounds as T cell activators, which inhibit the activity of DGKα and/or DGKζ, for treatment of viral infections and proliferative disorders, such as cancer.

WO2017/019723 A1 relates to azacyanoquinolinone compounds which may be useful as therapeutic agents for the treatment of central nervous system disorders associated with phosphodiesterase 9 (PDE9). It also relates to the use of the compounds compounds for treating neurological and psychiatric disorders.

WO2004/074218 describes MIF-inhibitors and multiple uses thereof, among others for treatment of cancer.

WO2007/109251 describes the use of TNFα inhibitors for treatment of diseases, among others for treatment of cancer.

WO 2012/142498 and WO2012/009649 describe MIF-inhibitors and multiple uses thereof, among others in cancer therapy. These patent applications claim an extremely high number of compounds. However, many of these theoretical compounds are not specifically disclosed.

However, the state of the art does not describe:
the specific substituted aminoquinolone compounds of general formula (I) of the present invention as described and defined herein, i.e. compounds having a 2-oxo-1,2-dihydroquinoline core bearing:
   in its 1-position a methyl- or an ethyl group,
   in its 3-position a cyano-, carbamoyl-, alkylcarbamoyl-, dialkylcarbamoyl- or alkoxycarbonyl group,
   in its 4-position a spirocyclic diamine group, which group is bound to the 2-oxo-1,2-dihydroquinoline core via a nitrogen atom of said spirocyclic diamine group, and
   as a substituent of said spirocyclic diamine group a phenyl-, naphthyl- or 5- to 10-membered heteroaryl group, which group is bound to a nitrogen atom of said spirocyclic diamine group,
   or stereoisomers, tautomers, N-oxides, hydrates, solvates, salts thereof, or mixtures of same, as described and defined herein, and as hereinafter referred to as “compounds of general formula (I)” or “compounds of the present invention”,
or their pharmacological activity.

It is desirable to provide novel compounds having prophylactic and therapeutic properties.

Accordingly, it is an object of the present invention to provide compounds and pharmaceutical compositions comprising these compounds used for prophylactic and therapeutic use in DGKα regulated disorders in a T cell immune-stimulatory or immune-modifying manner. DGKα regulated disorders comprise conditions with dysregulated immune responses, particularly in an immunologically suppressed tumor microenvironment in cancer, autoimmune diseases, viral infections as well as other disorders associated with aberrant DGKα signalling, e.g. fibrotic diseases. Said compounds can be used as sole agent or in combination with other active ingredients.

It has now been found, and this constitutes the basis of the present invention, that the compounds of the present invention have surprising and advantageous properties.

In particular, the compounds of the present invention have surprisingly been found to effectively inhibit the DGKα protein and enhance T-cell mediated immunity. Accordingly, they provide novel structures for the therapy of human and animal disorders, in particular of cancers, and may therefore be used for the treatment or prophylaxis of hyperproliferative disorders, such as cancer, for example.

### DESCRIPTION OF THE INVENTION

In accordance with a first aspect, the present invention covers compounds of general formula (I):

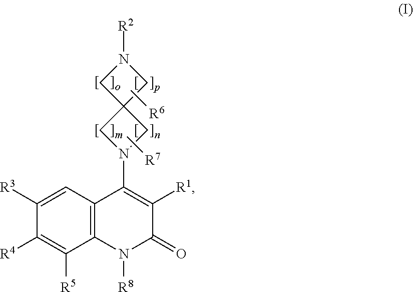

in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from
   C₁-C₆-alkyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from C₃-C₄-cycloalkyl, phenyl and 4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₆-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂alkyl, C₁-C₂-haloalkyl, cyano, hydroxy, C₁-C₂-alkoxy, C₃-C₄-cycloalkyl and —N(R⁹)(R¹⁰),
R³ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from cyano and hydroxy,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, N(R⁹)(R¹⁰), N(R¹⁶)(R¹⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl and —N(R⁹)(R¹⁰),
R⁵ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a C₁-C₄-alkyl group,
R⁷ represents a hydrogen atom, or a fluorine atom or a C₁-C₄-alkyl group,
R⁸ represents a group selected from methyl and ethyl,
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
      R⁹ and R¹⁰ together with the nitrogen to which they are attached represent a nitrogen containing 4- to 7-membered heterocycloalkyl group, wherein said nitrogen containing 4- to 7-membered heterocycloalkyl group is optionally substituted, one, two or three times, each substituent independently selected from a halogen atom or a group selected from
      C₁-C₄-alkyl, C₃-C₄-cycloalkyl, hydroxy and oxo,
      or
      two substituents, which are attached to the same carbon atom of said nitrogen containing 4- to 7-membered heterocycloalkyl group, together with the carbon atom to which they are attached, represent a 4- to 7-membered heterocycloalkyl group,
         wherein said 4- to 7-membered heterocycloalkyl group is optionally substituted, one or two times, each substituent independently selected from a halogen atom or a group selected from
         C₁-C₄-alkyl, C₃-C₄-cycloalkyl, C₁-C₄-haloalkyl, hydroxy and oxo,
R¹¹ represents a hydrogen atom or group selected from
   C₁-C₄-alkyl, C₁-C₄-hydroxyalkyl, C₁-C₄-haloalkyl, phenyl and
   5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from
   C₁-C₆-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a group selected from C₁-C₆-alkyl, C₁-C₆-haloalkyl, C₃-C₆-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl,
   C₁-C₂-haloalkyl, cyano, hydroxy, C₁-C₂-alkoxy, C₃-C₄-cycloalkyl and —N(R⁹)(R¹⁰),
R¹⁵ represents a hydrogen atom or a C₁-C₄-alkyl group,
R¹⁶ represents a hydrogen atom or a group selected from
   C₁-C₄-alkyl, C₃-C₄-cycloalkyl and C₂-C₄-haloalkyl,
R¹⁷ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
m and n represents, independently of each other, an integer selected from 1, 2 and 3, and
and p represents, independently of each other, an integer selected from 1, 2 and 3,
and stereoisomers, tautomers, N-oxides, hydrates, solvates, and salts thereof, and mixtures of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3 or 4, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one part, e.g. (C₁-C₂-alkoxy)-(C₁-C₆-alkyl)-, it is possible for a given part to be attached at any suitable position of said composite substituent, e.g. it is possible for the C₁-C₂-alkoxy part to be attached to any suitable carbon atom of the C₁-C₆-alkyl part of said (C₁-C₂-alkoxy)-(C₁-C₆-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethybutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group, more particularly 1 or 2 carbon atoms (“C₁-C₂alkyl”), e.g. a methyl or ethyl group.

1 or 2 carbon atoms (“C₁-C₂-alkyl”), e.g. a methyl or ethyl group.

The term “C₁-C₆-hydroxyalkyl” means a linear or branched, saturated, monovalent hydrocarbon group in which the term “C₁-C₆-alkyl” is defined supra, and in which 1 or 2 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl, 4-hydroxybutyl group, or an isomer thereof.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, it being understood that in the case in which said alkenyl group contains two double bonds, then it is possible for said double bonds to be conjugated with each other, or to form an allene. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group.

The term “C₂-C₆-alkynyl” means a linear or branched, monovalent hydrocarbon group which contains one triple bond, and which contains 2, 3, 4, 5 or 6 carbon atoms, particularly 2, 3 oder 4 carbon atoms (“C₂-C₄-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₃-C₆-cycloalkyl” means a saturated, monovalent, monocyclic hydrocarbon ring which contains 3, 4, 5 or 6 carbon atoms. Said C₃-C₆-cycloalkyl group is for example a cyclopropyl, cyclobutyl, cyclopentyl or cyclohexyl group. Particularly, said group has 3 or 4 carbon atoms (“C₃-C₄-cycloalkyl”), e.g. a cyclopropyl or cyclobutyl group.

The term “C₄-C₆-cycloalkenyl” means a monocyclic hydrocarbon ring which contains 4, 5 or 6 carbon atoms and one double bond. Particularly, said ring contains 5 or 6 carbon atoms (“C₅-C₆-cycloalkenyl”). Said C₄-C₆-cycloalkenyl group is for example, a monocyclic hydrocarbon ring, e.g. a cyclobutenyl, cyclopentenyl, cyclohexenyl or cycloheptenyll group.

The term “C₃-C₆-cycloalkyloxy” means a saturated, monovalent group of formula (C₃-C₆-cycloalkyl)-O—, in which the term “C₃-C₆-cycloalkyl” is as defined supra, e.g. a cyclopropyloxy, cyclobutyloxy, cyclopentyloxy or cyclohexyloxy group.

The term “4- to 7-membered heterocycloalkyl” means a monocyclic, saturated heterocycle with 4, 5, 6 or 7 ring atoms in total, which contains one or two identical or different ring heteroatoms from the series N, O and S.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

The term “5- to 7-membered heterocycloalkenyl” means a monocyclic, unsaturated, non-aromatic heterocycle with 5, 6 or 7 ring atoms in total, which contains one or two double bonds and one or two identical or different ring heteroatoms from the series N, O and S.

Said heterocycloalkenyl group is, for example, 4H-pyranyl, 2H-pyranyl, 2,5-dihydro-1H-pyrrolyl, [1,3]dioxolyl, 4H+[1,3,4]thiadiazinyl, 2,5-dihydrofuranyl, 2,3-dihydrofuranyl, 2,5-dihydrothio-phenyl, 2,3-dihydrothiophenyl, 4,5-dihydrooxazolyl or 4H-[1,4]thiazinyl.

The term “(4- to 7-membered heterocycloalkyl)oxy” means a monocyclic, saturated heterocycloalkyl of formula (4- to 7-membered heterocycloalkyl)-O— in which the term “4- to 7-membered heterocycloalkyl” is as defined supra.

The term “nitrogen containing 4- to 7-membered heterocycloalkyl group” means a monocyclic, saturated heterocycle with 4, 5, 6 or 7 ring atoms in total, which contains one ring nitrogen atom and optionally one further ring heteroatom from the series N, O and S.

Said nitrogen containing 4- to 7-membered heterocycloalkyl group, without being limited thereto, can be a 4-membered ring, such as azetidinyl, for example; or a 5-membered ring, such as pyrrolidinyl, imidazolidinyl, pyrazolidinyl, 1,2-oxazolidinyl, 1,3-oxazolidinyl or 1,3-thiazolidinyl, for example; or a 6-membered ring, such as piperidinyl, morpholinyl, thiomorpholinyl, piperazinyl, or 1,2-oxazinanyl, for example, or a 7-membered ring, such as azepanyl, 1,4-diazepanyl or 1,4-oxazepanyl, for example.

The term “heteroaryl” means a monovalent, monocyclic or bicyclic aromatic ring having 5, 6, 8, 9 or 10 ring atoms (a “5- to 10-membered heteroaryl” group), which contains at least one ring heteroatom and optionally one, two or three further ring heteroatoms from the series: N, O and/or S, and which is bound via a ring carbon atom.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₆-cycloalkyl”, means a cycloalkyl group having a finite number of carbon atoms of 3 to 6, i.e. 3, 4, 5 or 6 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₁-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₆” encompasses C₃, C₄, C₅, C₆, C₃-C₆, C₃-C₅, C₃-C₄, C₄-C₆, C₄-C₅, and C₅-C₆;

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I) having 1, 2, 3 or 4 deuterium atoms, particularly with 1, 2 or 3 deuterium atoms.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention.

The purification and the separation of such materials can be accomplished by standard techniques known in the art.

Preferred compounds are those which produce the more desirable biological activity. Separated, pure or partially purified isomers and stereoisomers or racemic or diastereomeric mixtures of the compounds of the present invention are also included within the scope of the present invention.

The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, the compounds of the present invention may contain an amide moiety and can exist as an amide, or an imidic acid, or even a mixture in any amount of the two tautomers, namely:

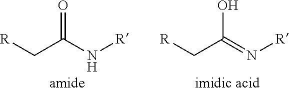

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the “Experimental Section”, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

The invention further includes all possible cyclodextrin clathrates, i.e alpha-, beta-, or gamma-cyclodextrins, hydroxypropyl-beta-cyclodextrins, methylbetacyclodextrins.

In accordance with a second embodiment of the first aspect, the present invention covers compounds of general formula (I), supra, in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂alkyl, C₁-C₂-haloalkyl, cyano, hydroxy, C₁-C₂alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from C₃-C₄-cycloalkyl, phenyl and
      4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₅-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R³ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, N(R⁹)(R¹⁰), N(R¹⁶)(R¹⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl and 5- or 6-membered heteroaryl group is optionally substituted, one or two times, each substituent independently selected from a halogen atom or a group selected from
   C₁-C₂alkyl, C₁-C₂-haloalkyl, cyano, hydroxy, C₁-C₂-alkoxy, C₃-C₄-cycloalkyl and —N(R⁹)(R¹⁰),
R⁵ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a C₁-C₄-alkyl group,
R⁷ represents a hydrogen atom, or a fluorine atom or a C₁-C₄-alkyl group,
R⁸ represents a group selected from methyl and ethyl,
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
      C₁-C₄-alkyl, C₃-C₄-cycloalkyl, hydroxy and oxo,
      or
      two substituents, which are attached to the same carbon atom of said nitrogen containing 4- to 7-membered heterocycloalkyl group, together with the carbon atom to which they are attached, represent a 4- to 7-membered heterocycloalkyl group,
         wherein said 4- to 7-membered heterocycloalkyl group is optionally substituted, one or two times, each substituent independently selected from a halogen atom or a group selected from
         C₁-C₄-alkyl, C₃-C₄-cycloalkyl, C₁-C₄-haloalkyl, hydroxy and oxo,
R¹¹ represents a hydrogen atom or group selected from
   C₁-C₄-alkyl, C₁-C₄-hydroxyalkyl, C₁-C₄-haloalkyl, phenyl and
   5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from
   C₁-C₄-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁵ represents a hydrogen atom or a C₁-C₄-alkyl group,
R¹⁶ represents a hydrogen atom or a group selected from
   C₁-C₄-alkyl, C₃-C₄-cycloalkyl and C₂-C₄-haloalkyl,
R¹⁷ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
m and n represents, independently of each other, an integer selected from 1, 2 and 3,
and
and p represents, independently of each other, an integer selected from 1, 2 and 3,
and stereoisomers, tautomers, N-oxides, hydrates, solvates, and salts thereof, and mixtures of same.

In accordance with a third embodiment of the first aspect, the present invention covers compounds of general formula (I), supra, in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from
   C₁-C₄-alkyl, C₁-C₄-haloalkyl, (C₁-C₂-alkoxy)-(C₁-C₄-alkyl)-, C₁-C₄-alkoxy, C₁-C₄-haloalkoxy and —N(R⁹)(R¹⁰),
   wherein said C₁-C₄-alkyl and C₁-C₄-alkoxy group is optionally substituted with a group selected from C₃-C₄-cycloalkyl, phenyl and
   4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from cyano and hydroxy,
R³ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy and —P(═O)(R¹⁴)₂,
   wherein said C₁-C₄-alkyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
R⁴ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy, N(R⁹)(R¹⁰), N(R¹⁶)(R¹⁷), —P(═O)(R¹⁴)₂ and (4- to 7-membered heterocycloalkyl)oxy,
   wherein said (4- to 7-membered heterocycloalkyl)oxy group is optionally substituted, one or two times, each substituent independently selected from a halogen atom or a group selected from
   C₁-C₂-alkyl, C₁-C₂haloalkyl, C₁-C₂-alkoxy, C₃-C₄-cycloalkyl and oxo, and
   wherein said C₁-C₄-alkyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
R⁵ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy, N(R⁹)(R¹⁰) and —P(═O)(R¹⁴)₂,
   wherein said C₁-C₄-alkyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
R⁶ represents a hydrogen atom,
R⁷ represents a hydrogen atom,
R⁸ represents a group selected from methyl and ethyl,
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
      C₁-C₄-alkyl, C₃-C₄-cycloalkyl, hydroxy and oxo,
      or
      two substituents, which are attached to the same carbon atom of said nitrogen containing 4- to 7-membered heterocycloalkyl group, together with the carbon atom to which they are attached, represent a 4- to 7-membered heterocycloalkyl group,
         wherein said 4- to 7-membered heterocycloalkyl group is optionally substituted, one or two times, each substituent independently selected from a halogen atom or a group selected from
         C₁-C₄-alkyl, C₃-C₄-cycloalkyl, C₁-C₄-haloalkyl, hydroxy and oxo,
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl,
   C₁-C₂-haloalkyl, cyano, hydroxy, C₁-C₂-alkoxy, C₃-C₄-cycloalkyl and —N(R⁹)(R¹⁰),
R¹⁵ represents a hydrogen atom or a C₁-C₄-alkyl group,
R¹⁶ represents a hydrogen atom or a group selected from
   C₁-C₄-alkyl, C₃-C₄-cycloalkyl and C₂-C₄-haloalkyl,
R¹⁷ represents a 4- to 7-membered heterocycloalkyl group,
   wherein said 4- to 7-membered heterocycloalkyl group is optionally substituted, one or two times, with a C₁-C₄-alkyl group,
m and n represents, independently of each other, an integer selected from 1, 2 and 3,
and
and p represents, independently of each other, an integer selected from 1, 2 and 3,
and stereoisomers, tautomers, N-oxides, hydrates, solvates, and salts thereof, and mixtures of same.

In accordance with a fourth embodiment of the first aspect, the present invention covers compounds of general formula (I), supra, in which:
R¹ represents a group selected from cyano —C(═O)NH₂, —C(═O)N(H)CH₃ and —C(═O)N(CH₃)₂,
R² represents a phenyl group,
   which group is optionally substituted, one, two, three or four times, each substituent independently selected from a halogen atom or a group selected from
   C₁-C₄-alkoxy, C₁-C₄-haloalkoxy and —N(R⁹)(R¹⁰),
R³ represents a hydrogen atom or a halogen atom or a —P(═O)(R¹⁴)₂ group,
R⁴ represents a hydrogen atom or a halogen atom or a group selected from N(R⁹)(R¹⁰), N(R¹⁶)(R¹⁷), —P(═O)(R¹⁴)₂ and (4- to 7-membered heterocycloalkyl)oxy, wherein said (4- to 7-membered heterocycloalkyl)oxy group is optionally substituted, one or two times, each substituent independently selected from a halogen atom or a group selected from
   C₁-C₂-alkyl, C₁-C₂-haloalkyl, C₁-C₂-alkoxy, C₃-C₄-cycloalkyl and oxo,
R⁵ represents a hydrogen atom or a halogen atom or a —P(═O)(R¹⁴)₂ group,
R⁶ represents a hydrogen atom,
R⁷ represents a hydrogen atom,
R⁸ represents a group selected from methyl and ethyl,
R⁹ and R¹⁰ represent, independently from each occurrence, a hydrogen atom or a group selected from
   C₁-C₄-alkyl and (C₁-C₄-alkoxy)-(C₂-C₄-alkyl)-,
   or
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
      C₁-C₄-alkyl and oxo,
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl,
   C₁-C₂-haloalkyl, cyano, hydroxy, C₁-C₂-alkoxy, C₃-C₄-cycloalkyl and —N(R⁹)(R¹⁰),
R¹⁶ represents a hydrogen atom or a group selected from
   C₁-C₄-alkyl, C₃-C₄-cycloalkyl and C₂-C₄-haloalkyl,
R¹⁷ represents a 4- to 7-membered heterocycloalkyl group,
   wherein said 4- to 7-membered heterocycloalkyl group is optionally substituted, one or two times, with a C₁-C₄-alkyl group,
m and n represents, independently of each other, an integer selected from 1, 2 and 3,
and
and p represents, independently of each other, an integer selected from 1, 2 and 3,
and stereoisomers, tautomers, N-oxides, hydrates, solvates, and salts thereof, and mixtures of same.

In accordance with a fifth embodiment of the first aspect, the present invention covers compounds of general formula (I), supra, in which:
R¹ represents a group selected from cyano and —C(═O)NH₂,
R² represents a phenyl group,
   which group is optionally substituted, one, two, three or four times, each substituent independently selected from a halogen atom or a group selected from
   C₁-C₄-alkoxy, C₁-C₄-haloalkoxy and —N(R⁹)(R¹⁰),
R³ represents a hydrogen atom,
R⁴ represents a hydrogen atom or a halogen atom or a group selected from N(R⁹)(R¹⁰), N(R¹⁶)(R¹⁷) and (4- to 7-membered heterocycloalkyl)oxy,
R⁵ represents a hydrogen atom,
R⁶ represents a hydrogen atom,
R⁷ represents a hydrogen atom,
R⁸ represents a methyl group,
R⁹ and R¹⁰ represent, independently from each occurrence, a hydrogen atom or a group selected from
   C₁-C₄-alkyl and (C₁-C₄-alkoxy)-(C₂-C₄-alkyl)-,
   or
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
      C₁-C₄-alkyl and oxo,
R¹⁶ represents a hydrogen atom or a C₁-C₄-alkyl group,
R¹⁷ represents a 4- to 7-membered heterocycloalkyl group,
   wherein said 4- to 7-membered heterocycloalkyl group is connected to the rest of the molecule via a carbon atom of the 4- to 7-membered heterocycloalkyl group,
m and n represents, independently of each other, an integer selected from 1, 2 and 3,
and
and p represents, independently of each other, an integer selected from 1, 2 and 3,
and stereoisomers, tautomers, N-oxides, hydrates, solvates, and salts thereof, and mixtures of same.

In accordance with a sixth embodiment of the first aspect, the present invention covers compounds of general formula (I), supra, in which:
R¹ represents a group selected from cyano and —C(═O)NH₂,
R² represents a phenyl group,
   which group is optionally substituted, one or two times, each substituent independently selected from a fluorine or a chlorine atom or a group selected from
   methoxy, trifluoromethoxy, morpholin-4-yl, N,N-dimethylamino and 2-oxopyrrolidin-1-yl,
R³ represents a hydrogen atom,
R⁴ represents a hydrogen atom or a bromine atom or a group selected from 4-methylpiperazin-1-yl, (2-methoxyethyl)(methyl)amino, methyl(tetrahydrofuran-3-yl)amino, (tetrahydrofuran-3-yl)oxy, (tetrahydro-2H-pyran-3-yl)oxy and (tetrahydro-2H-pyran-4-yl)oxy,
R⁵ represents a hydrogen atom,
R⁶ represents a hydrogen atom,
R⁷ represents a hydrogen atom,
R⁸ represents a methyl group,
m and n represents, independently of each other, an integer selected from 1 and 2,
represents, an integer of 1,
p represents an integer selected from 1, 2 and 3,
and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
   and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano —C(═O)NH₂, —C(═O)N(H)CH₃ and —C(═O)N(CH₃)₂,
   and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano and —C(═O)NH₂,
   and stereoisomers, tautomers, N-oxides, hydrates, solvates, and salts thereof, and mixtures of same.

In a further embodiment of the first aspect, the present invention covers compounds of formula (I), supra, in which:
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from
   C₁-C₆-alkyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from C₃-C₄-cycloalkyl, phenyl and
      4- to 7-membered heterocycloalkyl,
         wherein said 4- to 7-membered heterocycloalkyl group,
         is connected to the rest of the molecule via a carbon atom of said
         4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₆-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from C₁-C₄-alkyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from C₃-C₄-cycloalkyl, phenyl and
      4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R² represents a group selected from phenyl, naphthyl and 5- to 10-membered heteroaryl, which phenyl, naphthyl and 5- to 10-membered heteroaryl group is optionally substituted, one, two, three or four times, each substituent independently selected from a halogen atom or a group selected from
   C₁-C₄-alkyl, C₁-C₄-haloalkyl, (C₁-C₂-alkoxy)-(C₁-C₄-alkyl)-, C₁-C₄-alkoxy, C₁-C₄-haloalkoxy and —N(R⁹)(R¹⁰),
   wherein said C₁-C₄-alkyl and C₁-C₄-alkoxy group is optionally substituted with a group selected from C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
         and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₆-alkyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from C₃-C₄-cycloalkyl, phenyl and 4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from C₃-C₄-cycloalkyl, phenyl and
      4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
      C₁-C₄-alkyl, C₁-C₄-haloalkyl, (C₁-C₂-alkoxy)-(C₁-C₄-alkyl)-, C₁-C₄-alkoxy, C₁-C₄-haloalkoxy and —N(R⁹)(R¹⁰),
      wherein said C₁-C₄-alkyl and C₁-C₄-alkoxy group is optionally substituted with a group selected from C₃-C₄-cycloalkyl, phenyl and 4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
            and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkoxy, C₁-C₄-haloalkoxy and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one or two times, each substituent independently selected from a fluorine or a chlorine atom or a group selected from
   methoxy, trifluoromethoxy, morpholin-4-yl, N,N-dimethylamino and 2-oxopyrrolidin-1-yl,
      and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂alkyl, C₁-C₂-haloalkyl, cyano, hydroxy, C₁-C₂alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy and —P(═O)(R¹⁴)₂,
   wherein said C₁-C₄-alkyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group,
      is optionally substituted, one, two or three times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
         and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a —P(═O)(R¹⁴)₂ group,
   and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom,
   and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, N(R⁹)(R¹⁰), N(R¹⁶)(R¹⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₆-cycloalkyl and C₄-C₀-cycloalkenyl group is optionally substituted, one or two times, each substituent independently selected from a halogen atom or a C₁-C₄-alkyl group,
   and
   wherein said phenyl and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, N(R⁹)(R¹⁰), N(R¹⁶)(R¹⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said
      4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy, N(R⁹)(R¹⁰), N(R¹⁶)(R¹⁷), —P(═O)(R¹⁴)₂ and (4- to 7-membered heterocycloalkyl)oxy,
   wherein said (4- to 7-membered heterocycloalkyl)oxy group is optionally substituted, one or two times, each substituent independently selected from a halogen atom or a group selected from
   C₁-C₂-alkyl, C₁-C₂haloalkyl, C₁-C₂-alkoxy, C₃-C₄-cycloalkyl and oxo,
   and
   wherein said C₁-C₄-alkyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group is connected to the rest of the molecule via a carbon atom of said
      4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
         and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   N(R⁹)(R¹⁰), N(R¹⁶)(R¹⁷), —P(═O)(R¹⁴)₂ and (4- to 7-membered heterocycloalkyl)oxy,
      wherein said (4- to 7-membered heterocycloalkyl)oxy group is optionally substituted, one or two times, each substituent independently selected from a halogen atom or a group selected from
      C₁-C₂-alkyl, C₁-C₂haloalkyl, C₁-C₂-alkoxy, C₃-C₄-cycloalkyl and oxo,
         and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from N(R⁹)(R¹⁰), N(R¹⁶)(R¹⁷) and (4- to 7-membered heterocycloalkyl)oxy,
   and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a bromine atom or a group selected from 4-methylpiperazin-1-yl, (2-methoxyethyl)(methyl)amino, methyl(tetrahydrofuran-3-yl)amino, (tetrahydrofuran-3-yl)oxy, (tetrahydro-2H-pyran-3-yl)oxy and (tetrahydro-2H-pyran-4-yl)oxy,
   and stereoisomers, tautomers, N-oxides, hydrates, solvates, and salts thereof, and mixtures of same.

R⁵ represents a hydrogen atom or a halogen atom or a group selected from
C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
wherein said 4- to 7-membered heterocycloalkyl group and
5- to 7-membered heterocycloalkenyl group is connected to the rest of the molecule via a carbon atom of said 4- to 7-membered heterocycloalkyl group and 5- to 7-membered heterocycloalkenyl group,
and
wherein said 4- to 7-membered heterocycloalkyl group,
5- to 7-membered heterocycloalkenyl group and
(4- to 7-membered heterocycloalkyl)oxy group is optionally substituted, one, two or three times, each substituent independently selected from a halogen atom or a group selected from
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
wherein said C₁-C₆-alkyl, C₂-C₆-alkenyl, C₂-C₆-alkynyl and C₁-C₆-alkoxy group is optionally substituted with a group selected from
C₃-C₄-cycloalkyl, phenyl and 4- to 7-membered heterocycloalkyl,
   wherein said 4- to 7-membered heterocycloalkyl group,
   is connected to the rest of the molecule via a carbon atom of said 4- to 7-membered heterocycloalkyl group,
   and
   wherein said 4- to 7-membered heterocycloalkyl group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   which phenyl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
   and
   which C₃-C₄-cycloalkyl group is optionally substituted, one or two times, each substituent independently selected from a halogen atom or a group selected from
   cyano and hydroxy,
and
wherein said C₃-C₆-cycloalkyl and C₄-C₆-cycloalkenyl group is optionally substituted, one or two times, each substituent independently selected from a halogen atom or a C₁-C₄-alkyl group,
and
wherein said phenyl, phenoxy and 5- or 6-membered heteroaryl group is optionally substituted, one or two times, each substituent independently selected from a halogen atom or a group selected from
C₁-C₂-alkyl, C₁-C₂-haloalkyl, cyano, hydroxy, C₁-C₂-alkoxy, C₃-C₄-cycloalkyl and —N(R⁹)(R¹⁰),
   and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and
   5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group,
   5- to 7-membered heterocycloalkenyl group and
   (4- to 7-membered heterocycloalkyl)oxy group is optionally substituted, one, two or three times, each substituent independently selected from a halogen atom or a group selected from
   C₁-C₂-alkyl, C₁-C₂-haloalkyl, cyano, hydroxy, C₁-C₂alkoxy, C₃-C₄-cycloalkyl, —N(R⁹)(R¹⁰) and oxo,
   and
   wherein said C₁-C₄-alkyl, C₂-C₄-alkenyl, C₂-C₄-alkynyl and C₁-C₄-alkoxy group is optionally substituted with a group selected from
   C₃-C₄-cycloalkyl, phenyl and 4- to 7-membered heterocycloalkyl,
      wherein said 4- to 7-membered heterocycloalkyl group,
      is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₃-C₅-cycloalkyl and C₄-C₅-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from
   C₁-C₄-alkyl, C₁-C₄-haloalkyl, (C₁-C₂-alkoxy)-(C₁-C₄-alkyl)-, C₁-C₄-alkoxy, N(R⁹)(R¹⁰) and —P(═O)(R¹⁴)₂,
      wherein said C₁-C₄-alkyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group,
         is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
            and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a —P(═O)(R¹⁴)₂ group,
   and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom,
   and stereoisomers, tautomers, N-oxides, hydrates, solvates, and salts thereof, and mixtures of same.

In a further embodiment of the first aspect, the present invention covers compounds of formula (I), supra, in which:
R⁶ represents a hydrogen atom, or a fluorine atom or a C₁-C₄-alkyl group,
   and stereoisomers, tautomers, N-oxides, hydrates, solvates, and salts thereof, and mixtures of same.

In a further embodiment of the first aspect, the present invention covers compounds of formula (I), supra, in which:
R⁶ represents a hydrogen atom,
   and stereoisomers, tautomers, N-oxides, hydrates, solvates, and salts thereof, and mixtures of same.

In a further embodiment of the first aspect, the present invention covers compounds of formula (I), supra, in which:
R⁷ represents a hydrogen atom, or a fluorine atom or a C₁-C₄-alkyl group,
   and stereoisomers, tautomers, N-oxides, hydrates, solvates, and salts thereof, and mixtures of same.

In a further embodiment of the first aspect, the present invention covers compounds of formula (I), supra, in which:
R⁷ represents a hydrogen atom,
   and stereoisomers, tautomers, N-oxides, hydrates, solvates, and salts thereof, and mixtures of same.

In a further embodiment of the first aspect, the present invention covers compounds of formula (I), supra, in which:
R⁸ represents a group selected from methyl and ethyl,
   and stereoisomers, tautomers, N-oxides, hydrates, solvates, and salts thereof, and mixtures of same.

In a further embodiment of the first aspect, the present invention covers compounds of formula (I), supra, in which:
R⁸ represents a methyl group,
   and stereoisomers, tautomers, N-oxides, hydrates, solvates, and salts thereof, and mixtures of same.

In a further embodiment of the first aspect, the present invention covers compounds of formula (I), supra, in which:
R⁸ represents an ethyl group,
   and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
      R⁹ and R¹⁰ together with the nitrogen to which they are attached represent a nitrogen containing 4- to 7-membered heterocycloalkyl group, wherein said nitrogen containing 4- to 7-membered heterocycloalkyl group is optionally substituted, one, two or three times, each substituent independently selected from a halogen atom or a group selected from
      C₁-C₄-alkyl, C₃-C₄-cycloalkyl, hydroxy and oxo,
      or
      two substituents, which are attached to the same carbon atom of said nitrogen containing 4- to 7-membered heterocycloalkyl group, together with the carbon atom to which they are attached, represent a 4- to 7-membered heterocycloalkyl group,
         wherein said 4- to 7-membered heterocycloalkyl group is optionally substituted, one or two times, each substituent independently selected from a halogen atom or a group selected from
         C₁-C₄-alkyl, C₃-C₄-cycloalkyl, C₁-C₄-haloalkyl, hydroxy and oxo,
            and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
      and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ together with the nitrogen to which they are attached represent a nitrogen containing 4- to 7-membered heterocycloalkyl group, wherein said nitrogen containing 4- to 7-membered heterocycloalkyl group is optionally substituted, one, two or three times, each substituent independently selected from a halogen atom or a group selected from
C₁-C₄-alkyl, C₃-C₄-cycloalkyl, hydroxy and oxo,
or
two substituents, which are attached to the same carbon atom of said nitrogen containing 4- to 7-membered heterocycloalkyl group, together with the carbon atom to which they are attached, represent a 4- to 7-membered heterocycloalkyl group,
   wherein said 4- to 7-membered heterocycloalkyl group is optionally substituted, one or two times, each substituent independently selected from a halogen atom or a group selected from
   C₁-C₄-alkyl, C₃-C₄-cycloalkyl, C₁-C₄-haloalkyl, hydroxy and oxo,
      and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl and (C₁-C₄-alkoxy)-(C₂-C₄-alkyl)-,
   or
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
      C₁-C₄-alkyl and oxo,
         and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl and (C₁-C₄-alkoxy)-(C₂-C₄-alkyl)-,
      and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ together with the nitrogen to which they are attached represent a
   nitrogen containing 4- to 7-membered heterocycloalkyl group,
      wherein said nitrogen containing 4- to 7-membered heterocycloalkyl group is optionally substituted, one, two or three times, each substituent independently selected from a halogen atom or a group selected from
   C₁-C₄-alkyl and oxo,
      and stereoisomers, tautomers, N-oxides, hydrates, solvates, and salts thereof, and mixtures of same.

In a further embodiment of the first aspect, the present invention covers compounds of formula (I), supra, in which:
R¹¹ represents a hydrogen atom or group selected from
   C₁-C₄-alkyl, C₁-C₄-hydroxyalkyl, C₁-C₄-haloalkyl, phenyl and
   5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R¹² represents a hydrogen atom or a C₁-C₄-alkyl group,
   and stereoisomers, tautomers, N-oxides, hydrates, solvates, and salts thereof, and mixtures of same.

In a further embodiment of the first aspect, the present invention covers compounds of formula (I), supra, in which:
R¹³ represents a hydrogen atom or a group selected from
   C₁-C₆-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R¹³ represents a hydrogen atom or a group selected from
   C₁-C₄-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
         and stereoisomers, tautomers, N-oxides, hydrates, solvates, and salts thereof, and mixtures of same.

In a further embodiment of the first aspect, the present invention covers compounds of formula (I), supra, in which:
R¹⁴ represents a group selected from C₁-C₆-alkyl, C₁-C₆-haloalkyl, C₃-C₆-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl,
   C₁-C₂-haloalkyl, cyano, hydroxy, C₁-C₂-alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl,
   C₁-C₂-haloalkyl, cyano, hydroxy, C₁-C₂-alkoxy, C₃-C₄-cycloalkyl and —N(R⁹)(R¹⁰),
      and stereoisomers, tautomers, N-oxides, hydrates, solvates, and salts thereof, and mixtures of same.

In a further embodiment of the first aspect, the present invention covers compounds of formula (I), supra, in which:
R¹⁵ represents a hydrogen atom or a C₁-C₄-alkyl group,
   and stereoisomers, tautomers, N-oxides, hydrates, solvates, and salts thereof, and mixtures of same.

In a further embodiment of the first aspect, the present invention covers compounds of formula (I), supra, in which:
R¹⁶ represents a hydrogen atom or a group selected from
   C₁-C₄-alkyl, C₃-C₄-cycloalkyl and C₂-C₄-haloalkyl,

In a further embodiment of the first aspect, the present invention covers compounds of formula (I), supra, in which:
R¹⁶ represents a hydrogen atom or a C₁-C₄-alkyl group,
   and stereoisomers, tautomers, N-oxides, hydrates, solvates, and salts thereof, and mixtures of same.

In a further embodiment of the first aspect, the present invention covers compounds of formula (I), supra, in which:
R¹⁷ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
      and stereoisomers, tautomers, N-oxides, hydrates, solvates, and salts thereof, and mixtures of same.

In a further embodiment of the first aspect, the present invention covers compounds of formula (I), supra, in which:
R¹⁷ represents a 4- to 7-membered heterocycloalkyl group,
   wherein said 4- to 7-membered heterocycloalkyl group is optionally substituted, one or two times, with a C₁-C₄-alkyl group,
   wherein said 4- to 7-membered heterocycloalkyl group is connected to the rest of the molecule via a carbon atom of the 4- to 7-membered heterocycloalkyl group,
      and stereoisomers, tautomers, N-oxides, hydrates, solvates, and salts thereof, and mixtures of same.

In a further embodiment of the first aspect, the present invention covers compounds of formula (I), supra, in which:
R¹⁷ represents a 4- to 7-membered heterocycloalkyl group,
   wherein said 4- to 7-membered heterocycloalkyl group is connected to the rest of the molecule via a carbon atom of the 4- to 7-membered heterocycloalkyl group,
      and stereoisomers, tautomers, N-oxides, hydrates, solvates, and salts thereof, and mixtures of same.

In a further embodiment of the first aspect, the present invention covers compounds of formula (I), supra, in which:
m and n represents, independently of each other, an integer selected from 1, 2 and 3,
   and stereoisomers, tautomers, N-oxides, hydrates, solvates, and salts thereof, and mixtures of same.

In a further embodiment of the first aspect, the present invention covers compounds of formula (I), supra, in which:
m and n represents, independently of each other, an integer selected from 1 and 2,
   and stereoisomers, tautomers, N-oxides, hydrates, solvates, and salts thereof, and mixtures of same.

In a further embodiment of the first aspect, the present invention covers compounds of formula (I), supra, in which:
o and p represents, independently of each other, an integer selected from 1, 2 and 3,
   and stereoisomers, tautomers, N-oxides, hydrates, solvates, and salts thereof, and mixtures of same.

In a further embodiment of the first aspect, the present invention covers compounds of formula (I), supra, in which:
o represents, an integer of 1,
   and stereoisomers, tautomers, N-oxides, hydrates, solvates, and salts thereof, and mixtures of same.

In a further embodiment of the first aspect, the present invention covers compounds of formula (I), supra, in which:
p represents an integer selected from 1, 2 and 3,
   and stereoisomers, tautomers, N-oxides, hydrates, solvates, and salts thereof, and mixtures of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers the compounds of general formula (I) which are disclosed in the Example Section of this text, infra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit DGKα and it is possible therefore that said compounds be used for the treatment or prophylaxis of diseases, preferably conditions with dysregulated immune responses, particularly cancer or other disorders associated with aberrant DGKα signaling, in humans and animals.

Disorders and conditions particularly suitable for treatment with an DGKα inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention are used in combination with radiation: i.e. radiation treatment sensitizes cancers to anti-tumor immune responses by induction of tumor cell death and subsequent presentation of tumor neoantigens to tumor-reactive Tcells. As DGKα is enhancing the antigen specific activation of T cells, the overall effect results in a much stronger cancer cell attack as compared to irradiation treatment alone.

Thus, the present invention also provides a method of killing a tumor, wherein conventional radiation therapy is employed previous to administering one or more of the compounds of the present invention.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists. PD-1, along with its ligands PD-L1 and PD-L2, function as negative regulators of T cell activation. DGKα suppresses immune cell function. PD-L1 is overexpressed in many cancers and overexpression of PD-1 often occurs concomitantly in tumor infiltrating T cells. This results in attenuation of T cell activation and evasion of immune surveillance, which contributes to impaired antitumor immune responses. (Keir M E et al. (2008) Annu. Rev. Immunol. 26:677).

In accordance with a further aspect, the present invention covers combinations comprising one or more of the compounds of general formula (I), as described herein, or stereoisomers, tautomers, N-oxides, hydrates, solvates, and salts thereof, particularly pharmaceutically acceptable salts thereof, or mixtures of same, and one or more immune checkpoint inhibitors. Preferably, the immune checkpoint inhibitor is a aPD-1/-L1 axis antagonist.

The compounds of the invention can further be combined with chimeric antigen receptor T cells (CAR-T cells), such as Axicabtagen-Ciloleucel or Tisagenlecleucel. The activity of CAR-T cells can be suppressed by the tumor micro environment (TME). Knock out of DGKα by techniques such as Crispr had been shown to enhance CAR-T cell activity in a suppressive TME (Mol. Cells 2018; 41(8): 717-723).

In accordance with a further aspect, the present invention covers combinations comprising one or more compounds of general formula (I), as described herein, or stereoisomers, tautomers, N-oxides, hydrates, solvates, and salts thereof, particularly pharmaceutically acceptable salts thereof, or mixtures of same, with chimeric antigen receptor T cells, (CAR-T cells), CAR-NKT cells or CAR-NK cells.

Preferably, the chimeric antigen receptor T cells (CAR-T cells) are Axicabtagen-Ciloleucel or Tisagenlecleucel.

The present invention further provides the use of the compounds according to the invention for expansion of T cells including CAR-T and tumor infiltrated lymphocytes ex-vivo. Inhibition of DGKα was shown to reactivate ex vivo treated T cells (Prinz et al. (2012) J. Immunol).

In accordance with a further aspect, the present invention covers compounds of general formula (I), as described herein, or stereoisomers, tautomers, N-oxides, hydrates, solvates, and salts thereof, particularly pharmaceutically acceptable salts thereof, or mixtures of same, for use in the expansion of T cells including CAR-T cells, CAR-NKT cells or CAR-NK cells and tumor infiltrated lymphocytes ex-vivo.

Hence, the present invention also relates to the use of the compounds according to the invention for the expansion of T cells, including CAR-T cell, CAR-NKT cells or CAR-NK cells and tumor infiltrated lymphocytes, ex-vivo.

The present invention also comprises an ex-vivo method for the expansion of T cells, including CAR-T cells, CAR-NKT cells or CAR-NK cells and tumor infiltrated lymphocytes, contacting said T cells with compounds according to the invention.

The compounds of the invention can further be combined with inhibitors of DGKζ, such as those inhibitors of DGKζ disclosed in WO2020/006016 and WO2020/006018. As DGKζ in T cells operates in a similar fashion as DGKα, a dual inhibition profoundly enhances T cell effector functions compared with cells with deletion of either DGK isoform alone or wild-type cells (Riese et al., Cancer Res 2013, 73(12), 3566).

Compounds of the present invention can be utilized to inhibit, block, reduce or decrease DGKα activity resulting in the modulation of dysregulated immune responses e.g. to block immunosuppression and increase immune cell activation and infiltration in the context of cancer and cancer immunotherapy that will eventually lead to reduction of tumour growth.

This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating a variety of other disorders wherein DGKα is involved such as, but not limited to, disorders with dysregulated immune responses, inflammation, vaccination for infection & cancer, viral infections, obesity and diet-induced obesity, adiposity, metabolic disorders, fibrotic disorders, cardiac diseases and lymphoproliferative disorders.

These disorders have been well characterized in humans, but also exist with a similar etiology in other mammals, and can be treated by administering pharmaceutical compositions of the present invention.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer or conditions with dysregulated immune responses or other disorders associated with aberrant DGKα signaling.

The pharmaceutical activity of the compounds according to the invention can be explained by their activity as DGKα inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer or conditions with dysregulated immune responses or other disorders associated with aberrant DGKα signaling, particularly liquid and solid tumours.

In accordance with a further aspect, the present invention covers the compounds of general formula (I), as described supra, or stereoisomers, tautomers, N-oxides, hydrates, solvates, and salts thereof, particularly pharmaceutically acceptable salts thereof, or mixtures of same, for the use of treatment or prophylaxis of diseases, in particular cancer or conditions with dysregulated immune responses or other disorders associated with aberrant DGKα signaling, particularly liquid and solid tumours.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer or conditions with dysregulated immune responses or other disorders associated with aberrant DGKα signaling, particularly liquid and solid tumours.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer or conditions with dysregulated immune responses or other disorders associated with aberrant DGKα signaling, particularly liquid and solid tumours.

In accordance with a further aspect, the present invention covers a method of treatment or prophylaxis of diseases, in particular cancer or conditions with dysregulated immune responses or other disorders associated with aberrant DGKα signaling, particularly liquid and solid tumours, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit)),
capsule materials (for example gelatine, hydroxypropylmethylcellulose),
synthetic polymers (for example polylactides, polyglycolides, polyacrylates, polymethacrylates (such as, for example, Eudragit), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of cancer or conditions with dysregulated immune responses or other disorders associated with aberrant DGKα signaling, particularly liquid and solid tumours.

Particularly, the present invention covers a pharmaceutical combination, which comprises:
one or more first active ingredients, in particular compounds of general formula (I) as defined supra, and
one or more further active ingredients, in particular in particular immune checkpoint inhibitors.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Based upon standard laboratory techniques known to evaluate compounds useful for the treatment of cancer or conditions with dysregulated immune responses or other disorders associated with aberrant DGKα signaling, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Syntheses of Compounds

The compounds according to the invention of general formula (I) can be prepared according to the following schemes 1-9. The schemes and procedures described below illustrate synthetic routes to the compounds of general formula (I) of the invention and are not intended to be limiting. It is clear to the person skilled in the art that the order of transformations as exemplified in schemes 1-9 can be modified in various ways. The order of transformations exemplified in these schemes is therefore not intended to be limiting. In addition, interconversion of any of the substituents, R¹, R², R³, R⁴, R⁵, R⁶, R⁷ or R⁸, can be achieved before and/or after the exemplified transformations. These modifications can be such as the introduction of protecting groups, cleavage of protecting groups, reduction or oxidation of functional groups, halogenation, metalation or substitution known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 4^{th} edition, Wiley 2006). Specific examples are described in the subsequent paragraphs.

Isatoic anhydrides 1 are widely available from commercial suppliers or described in the literature. For example the isatoic anhydrides 1 can be prepared from 2-aminobenzoic acids 2 (in analogy to the procedure in Tetrahedron Lett. 2014, 55, 3607-3609) using triphosgene in an organic solvent such as THF or 1,4-dioxane or (in analogy to the procedure in Tetrahedron Lett. 2013, 54, 6897-6899) using di-tert-butyl dicarbonate and a base such as NaOH followed by treatment with 2-chloromethylpyridinium iodide and subsequent acidic workup (Scheme 1).

Alternatively, preparation of the isatoic anhydrides 1 can also be achieved (for example in analogy to the procedure in J. Org. Chem. 2014, 79, 4196-4200) using Pd-catalyzed oxidative double carbonylation of o-iodoanilines 3.

The obtained isatoic anhydrides 1 can then be alkylated at the nitrogen to obtain compounds of the general formula 4. Typically an alkylating agent such as for example an alkylbromide, alkyliodide or alkylsulfonate, a base such as disopropylethylamine, K₂CO₃ or KOtBu in an organic solvent is used.

Alternatively the alkylated isatoic anhydrides 4 can be prepared directly from secondary anilines (in analogy to the procedure in Tetrahedron Lett. 2014, 55, 3607-3609) using triphosgene in an organic solvent such as THF or 1,4-dioxane or (in analogy to the procedure in Tetrahedron Lett. 2013, 54, 6897-6899) using di-tert-butyl dicarbonate and a base such as NaOH followed by treatment with 2-chloromethylpyridinium iodide and subsequent acidic workup.

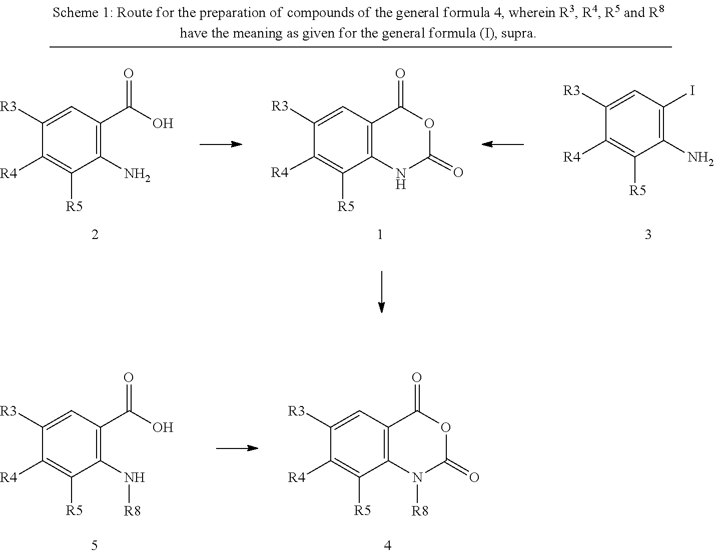

Isatoic anhydrides 4 can be converted to the corresponding quinolones 7 using ethyl acetate derivatives 6 such as for example ethylcyano acetate (for R¹═CN), a base such as for example triethylamine in an organic solvent such as for example THF (Scheme 2).

Hydroxy quinolones 7 can be converted to the corresponding halides 8 using for example phosphoryl chloride (X=chloro) or phosphoryl bromide (X=bromo).

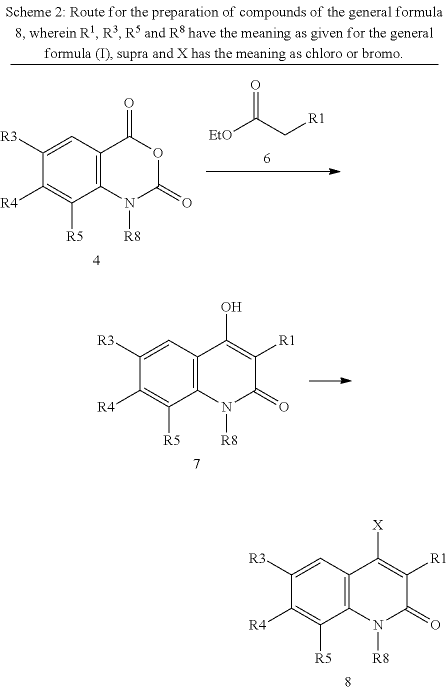

Halides of the general formula 8 can be reacted with amines 9 to yield compounds of the general formula 10 (Scheme 3). Typically the reaction is performed in an organic solvent such as for example isopropanol and a base such as for example diisopropylethylamine or triethylamine.

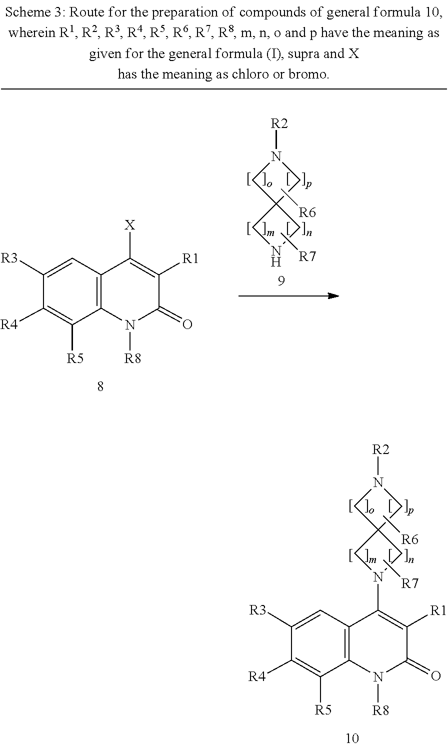

Nitriles of the general formula 11 can be converted to the amides of the general formula 12 (Scheme 4). Typically the reaction is performed with palladium(II)acetate and acetaldoxime in an organic solvent such as for example ethanol (see for example J. Med. Chem. 2016, 59, 6281 ff, Degorce et al.).

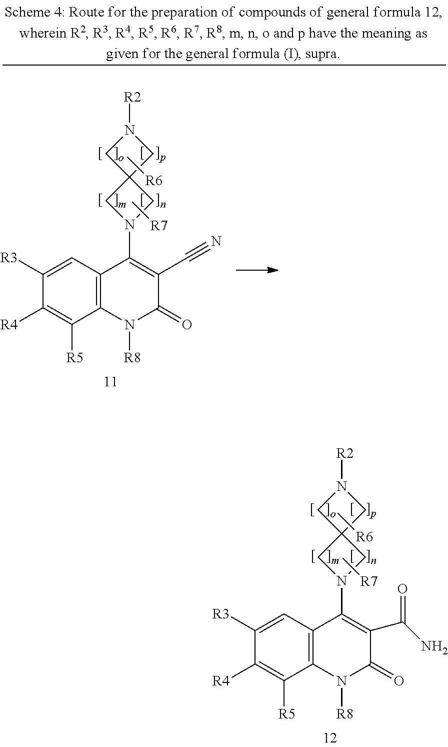

Chinolones of general formular 10 can also be prepared from chloro or bromo-substituted chinolones of general formula 8 through reaction with spirocyclic amines of general formula 13, wherein M represents a BOC-protecting group, and a base such as triethylamine or DiPEA to give intermediates of general formula 14. Many amines of the general formula 13 are commercially available or described in the literature. Several spirocyclic building blocks consisting of 5/6, 6/6, 5/5, 4/5 and 6/4 ring systems containing BOC protection groups on either side are commercially available. The BOC-protecting group can be cleaved with a strong acid such as TFA or HCl in dioxane to provide the free amine 15. In a late-stage functionalization approach the amine intermediate 15 can be coupled to various bromides under Buchwald-Hartwig conditions by using for example chloro(2-dicyclohexylphosphino-2,4,6-triisopropyl-1,1-biphenyl)[2-(2-amino-1,1-biphenyl)]palladium(II) as a Pd-catalyst, Cs₂CO₃ as a base in 1,4-dioxane as a solvent at elevated temperatures, to yield compounds of general formula 10.

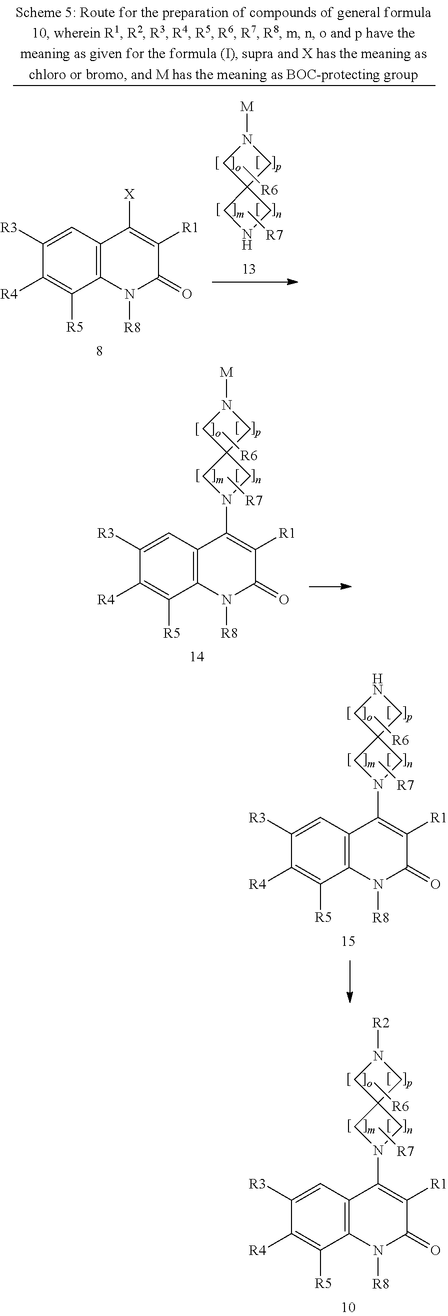

Isatoic anhydrides 4 can be converted to the corresponding quinolones 16 using diethylmalonate, a base such as for example triethylamine in an organic solvent such as for example THF (Scheme 6).

Hydroxy quinolones 16 can be converted to the corresponding halides 17 using for example phosphoryl chloride (X=chloro) or phosphoryl bromide (X=bromo).

Halides of the general formula 17 can be reacted with amines 9 to yield compounds of the general formula 18. Typically the reaction is performed in an organic solvent such as for example isopropanol and with a base such as for example diisopropylethylamine.

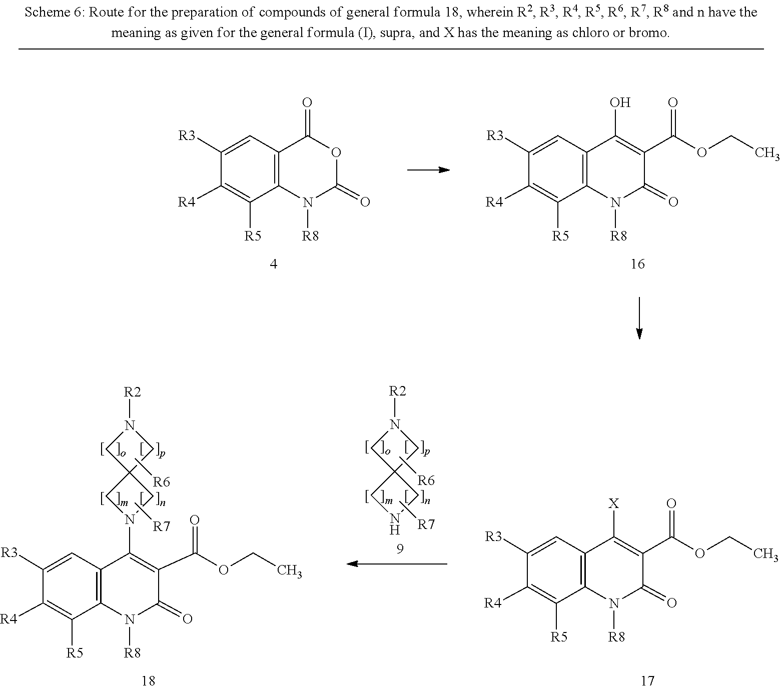

Esters of the general formula 18 can be converted to the corresponding carboxylic acids 19 using classical ester hydrolysis conditions. Typically LIOH, KOH or NaOH in water/ethanol/THF at elevated temperatures is used for this reaction (Scheme 7).

The carboxylic acids of the general formula 19 and amines of general formula 20 can be converted to the corresponding amides 21 using standard amide forming reaction known to the person skilled in the art. For a review see for example Chem. Rev. 2011, 111, 6557-6602. Compounds of general formula 20 are commercially available or described in the literature.

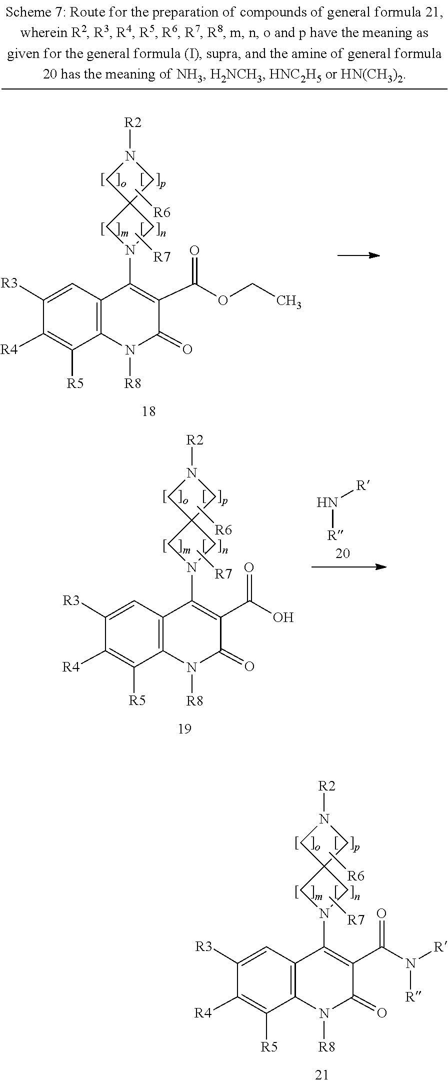

Alternatively, a spirocycle such as 26 (Scheme 8) can be prepared by alkylation of nitrile 22 using LDA and gaseous formaldehyde followed by switching the protecting group from benzyl (23) to BOO to give intermediated 24. Tosylation of the primary alcohol to give 25 and reduction of the nitrile to the primary amine using LAH results in cyclization to the desired azetidine 26 (WO2007030061).

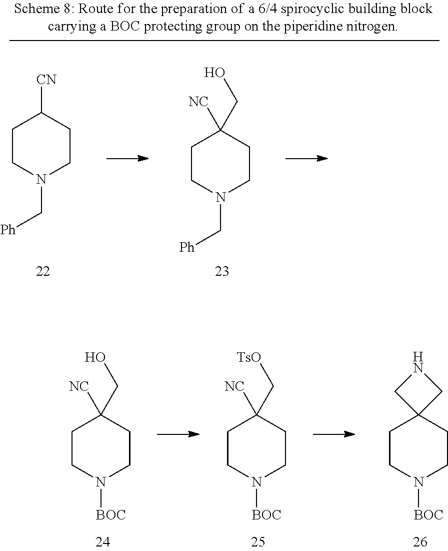 [SMILES: N#CC1CCN(Cc2ccccc2)CC1 ; N#CC1(CO)CCN(Cc2ccccc2)CC1 ; CC(C)(C)OC(=O)N1CCC(C#N)(CO)CC1 ; Cc1ccc(S(=O)(=O)OCC2(C#N)CCN(C(=O)OC(C)(C)C)CC2)cc1 ; CC(C)(C)OC(=O)N1CCC2(CC1)CNC2]

Spirocyclic amines of general formula 9, wherein M represents a BOO-protecting group, can be prepared from commercial BOO-protected amines of general formula 13 by Buchwald-Hartwig coupling followed by cleavage of the BOO group of intermediate 27 (Scheme 9).

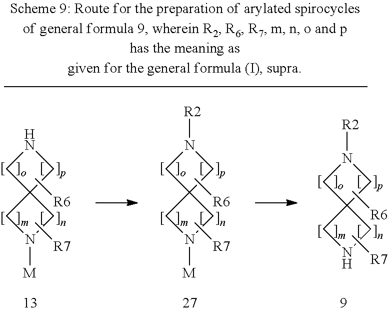

In accordance with a second aspect, the present invention covers methods of preparing compounds of general formula (I), said methods comprising the step of allowing an intermediate compound of general formula (II):

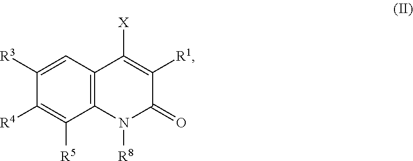

in which R¹, R³, R⁴, R⁵ and R⁸ are as defined for the compound of general formula (I) as defined supra, and X has the meaning of chloro or bromo,
to react with a compound of general formula (III):

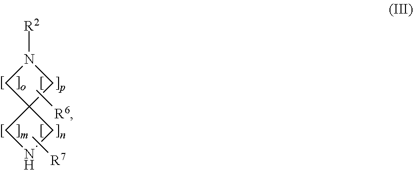

in which R², R⁶, R⁷, m, n, o and p are as defined for the compound of general formula (I) as defined supra,
thereby giving a compound of general formula (I):

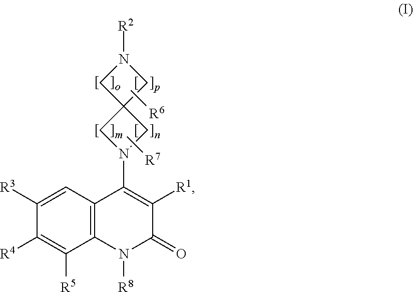

in which R¹, R², R³, R⁴, R⁵, R⁶, R⁷, R⁸, m, n, o and p are as defined supra.

In accordance with a second embodiment of the second aspect, the present invention covers methods of preparing compounds of general formula (I), said methods comprising the step of allowing an intermediate compound of general formula (IV):

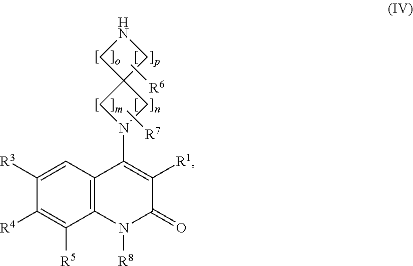

in which R¹, R³, R⁴, R⁵, R⁶, R⁷, R⁸, m, n, o and p are as defined for the compound of general formula (I) as defined supra, and X has the meaning of chloro or bromo,
to react with a compound of general formula (V):

R²—Br (V),

in which R² is as defined for the compound of general formula (I) as defined supra,
in the presence of a Pd-catalyst, such as for example chloro(2-dicyclohexylphosphino-2,4,6-triisopropyl-1,1-biphenyl)[2-(2-amino-1,1-biphenyl)]palladium(II),
thereby giving a compound of general formula (I):

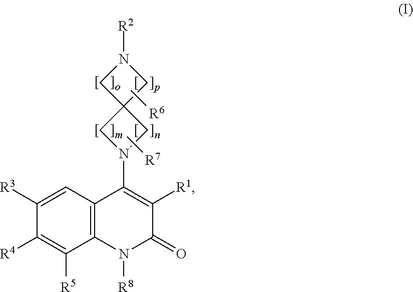

in which R¹, R², R³, R⁴, R⁵, R⁶, R⁷, R⁸, m, n, o and p are as defined supra.

In accordance with a third embodiment of the second aspect, the present invention covers methods of preparing compounds of general formula (I-b), which are compounds of general formula (I) in which R², R³, R⁴, R⁵, R⁶, R⁷, R⁸, m, n, o and p are as defined for the compound of general formula (I) as defined supra, and R¹ represents a carbamoyl group, said methods comprising the step of allowing a compound of general formula (I-a):

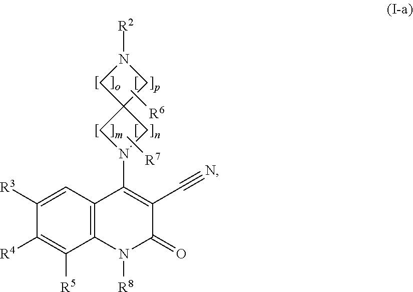

which is a compound of general formula (I) in which R², R³, R⁴, R⁵, R⁶, R⁷, R⁸, m, n, o and p are as defined for the compound of general formula (I) as defined supra, and R¹ represents a cyano group,
to react with palladium(II)acetate and acetaldoxime,
thereby giving a compound of general formula (I-b):

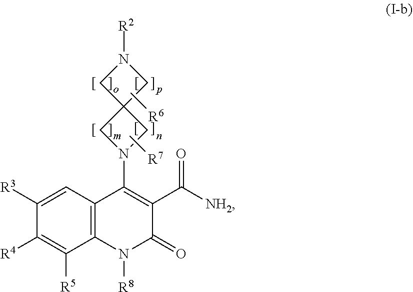

in which R², R³, R⁴, R⁵, R⁶, R⁷, R⁸, m, n, o and p are as defined supra, and R¹ represents a carbamoyl group.

In accordance with a fourth embodiment of the second aspect, the present invention covers methods of preparing compounds of general formula (I-d), which are compounds of general formula (I) in which R², R³, R⁴, R⁵, R⁶, R⁷, R⁸, m, n, o and p are as defined for the compound of general formula (I) as defined supra, and R¹ represents a —C(═O)NH₂, —C(═O)N(H)CH₃, —C(═O)N(H)C₂H₅ or —C(═O)N(CH₃)₂ group, said methods comprising the step of allowing a compound of general formula (I-c):

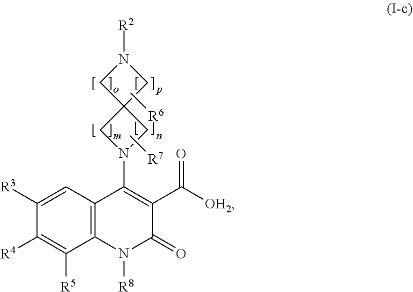

which is a compound of general formula (I) in which R², R³, R⁴, R⁵, R⁶, R⁷, R⁸, m, n, o and p are as defined for the compound of general formula (I) as defined supra, and R¹ represents a carboxyl group,
to react with a compound of general formula (VI):

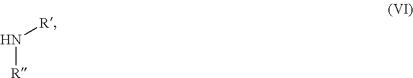

which compound is NH₃, H₂NCH₃, H₂NCH₂CH₃ or HN(CH₃)₂, or salts thereof,
thereby giving a compound of general formula (I-d):

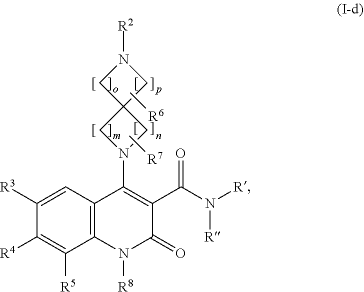

which is a compound of general formula (I) in which R², R³, R⁴, R⁵, R⁶, R⁷, R⁸, m, n, o and p are as defined supra, and R¹ represents a group —C(═O)NH₂, —C(═O)N(H)CH₃, —C(═O)N(H)C₂H₅ or —C(═O)N(CH₃)₂.

In accordance with a third aspect, the present invention covers methods of preparing compounds of general formula (I), said methods comprising the step of allowing an intermediate compound of general formula (II):

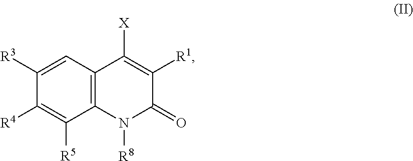

in which R¹, R³, R⁴, R⁵ and R⁸ are as defined for the compound of general formula (I) as defined supra, and X has the meaning of chloro or bromo,
to react with a compound of general formula (III):

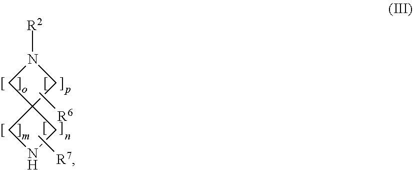

in which R², R⁶, R⁷, m, n, o and p are as defined for the compound of general formula (I) as defined supra,
thereby giving a compound of general formula (I):

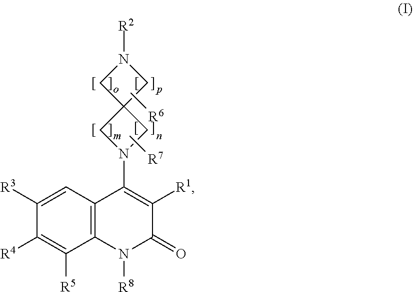

in which R¹, R², R³, R⁴, R⁵, R⁶, R⁷, R⁸, m, n, o and p are as defined supra,
then optionally converting said compound into solvates, salts and/or solvates of such salts using the corresponding (i) solvents and/or (ii) bases or acids.

In accordance with a second embodiment of the third aspect, the present invention covers methods of preparing compounds of general formula (I), said methods comprising the step of allowing an intermediate compound of general formula (IV):

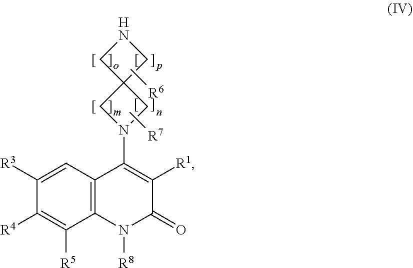

in which R¹, R³, R⁴, R⁵, R⁶, R⁷, R⁸, m, n, o and p are as defined for the compound of general formula (I) as defined supra, and X has the meaning of chloro or bromo,
to react with a compound of general formula (V):

R²—Br (V),

in which R² is as defined for the compound of general formula (I) as defined supra,
in the presence of a Pd-catalyst, such as for example chloro(2-dicyclohexylphosphino-2,4,6-triisopropyl-1,1-biphenyl)[2-(2-amino-1,1-biphenyl)]palladium(II),
thereby giving a compound of general formula (I):

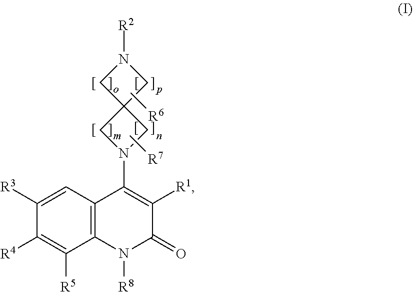

in which R¹, R², R³, R⁴, R⁵, R⁶, R⁷, R⁸, m, n, o and p are as defined supra,
then optionally converting said compound into solvates, salts and/or solvates of such salts using the corresponding (i) solvents and/or (ii) bases or acids.

In accordance with a third embodiment of the third aspect, the present invention covers methods of preparing compounds of general formula (I-b), which are compounds of general formula (I) in which R², R³, R⁴, R⁵, R⁶, R⁷, R⁸, m, n, o and p are as defined for the compound of general formula (I) as defined supra, and R¹ represents a carbamoyl group, said methods comprising the step of allowing a compound of general formula (I-a):

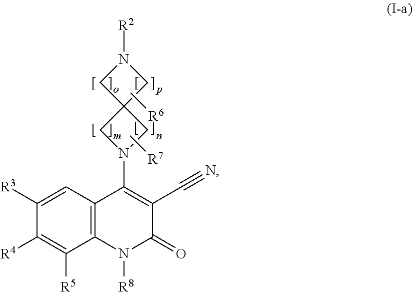

which is a compound of general formula (I) in which R², R³, R⁴, R⁵, R⁶, R⁷, R⁸, m, n, o and p are as defined for the compound of general formula (I) as defined supra, and R¹ represents a cyano group,
to react with palladium(II)acetate and acetaldoxime,
thereby giving a compound of general formula (I-b):

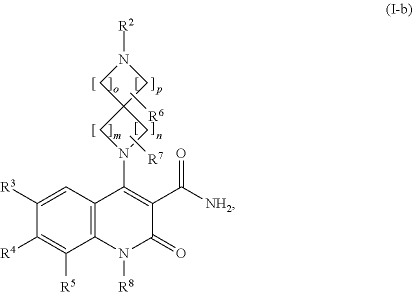

in which R², R³, R⁴, R⁵, R⁶, R⁷, R⁸, m, n, o and p are as defined supra, and R¹ represents a carbamoyl group,
then optionally converting said compound into solvates, salts and/or solvates of such salts using the corresponding (i) solvents and/or (ii) bases or acids.

In accordance with a fourth embodiment of the third aspect, the present invention covers methods of preparing compounds of general formula (I-d), which are compounds of general formula (I) in which R², R³, R⁴, R⁵, R⁶, R⁷, R⁸, m, n, o and p are as defined for the compound of general formula (I) as defined supra, and R¹ represents a —C(═O)NH₂, —C(═O)N(H)CH₃, —C(═O)N(H)C₂H₅ or —C(═O)N(CH₃)₂ group, said methods comprising the step of allowing a compound of general formula (I-c):

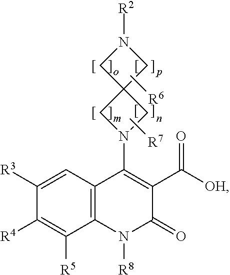

which is a compound of general formula (I) in which R², R³, R⁴, R⁵, R⁶, R⁷, R⁸, m, n, o and p are as defined for the compound of general formula (I) as defined supra, and R¹ represents a carboxyl group,
to react with a compound of general formula (VI):

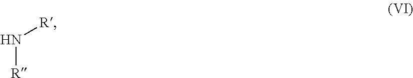

which compound is NH₃, H₂NCH₃, H₂NCH₂CH₃ or HN(CH₃)₂, or salts thereof,
thereby giving a compound of general formula (I-d):

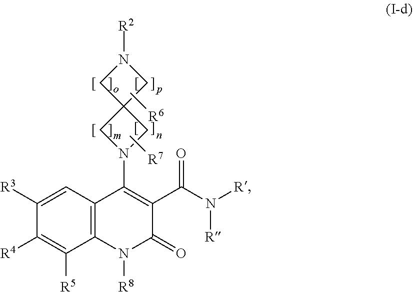

which is a compound of general formula (I) in which R², R³, R⁴, R⁵, R⁶, R⁷, R⁸, m, n, o and p are as defined supra, and R¹ represents a group —C(═O)NH₂, —C(═O)N(H)CH₃, —C(═O)N(H)C₂H₅ or —C(═O)N(CH₃)₂,
then optionally converting said compound into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

In accordance with a fourth aspect, the present invention covers the use of intermediate compounds for the preparation of a compound of general formula (I) as defined supra.

Particularly, the inventions covers the use of intermediate compounds of general formula (II):

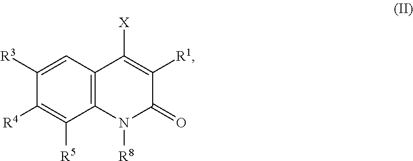

in which R¹, R³, R⁴, R⁵ and R⁸ are as defined for the compound of general formula (I) as defined supra, and X has the meaning of chloro or bromo, for the preparation of a compound of general formula (I) as defined supra.

Particularly, the inventions covers the use of intermediate compounds of general formula (III):

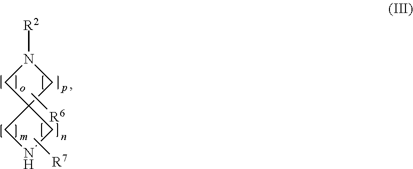

in which R², R⁶, R⁷, m, n, o and p are as defined for the compound of general formula (I) as defined supra, for the preparation of a compound of general formula (I) as defined supra.

Particularly, the inventions covers the use of intermediate compounds of general formula (IV):

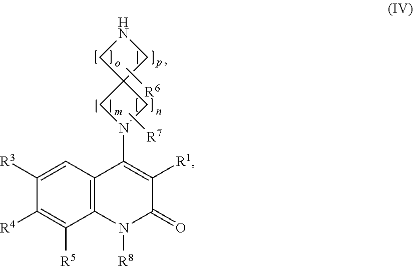

in which R¹, R³, R⁴, R⁵, R⁶, R⁷, R⁸, m, n, o and p are as defined for the compound of general formula (I) as defined supra, for the preparation of a compound of general formula (I) as defined supra.

Particularly, the inventions covers the use of intermediate compounds of general formula (V):

R²—Br (V)

in which R² is as defined for the compound of general formula (I) as defined supra, for the preparation of a compound of general formula (I) as defined supra.

Particularly, the inventions covers the use of intermediate compounds of general formula (VI):

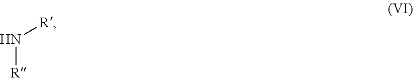

which compounds are NH₃, H₂NCH₃, H₂NCH₂CH₃ or HN(CH₃)₂, or salts thereof, for the preparation of a compound of general formula (I) as defined supra.

The present invention covers the use of intermediate compounds which are disclosed in the Example Section of this text, infra.

The present invention covers any sub-combination within any embodiment or aspect of the present invention of intermediate compounds of general formulae (II), (III), (IV), (V) and (VI), supra.

### DESCRIPTION OF THE FIGURES

FIG. 1: human DGKα M1 to S735 plus C-terminal Flag-Tag, DGKa_hu_1, as described under SEQ ID No. 1.

FIG. 2: human DGKα M1 to S735 plus N-terminal Avi-Tag and C-terminal Flag-Tag, DGKa_hu_1Avi, as described under SEQ ID No. 2.

FIG. 3: SIINFEKL amino acid sequence, as described under SEQ ID No. 3.

FIG. 4: GCCACC DNA sequence

FIG. 5: Flag-Tag sequence, as described under SEQ ID No. 4.

FIG. 6: OVA-30 peptide sequence, as described under SEQ ID No. 5.

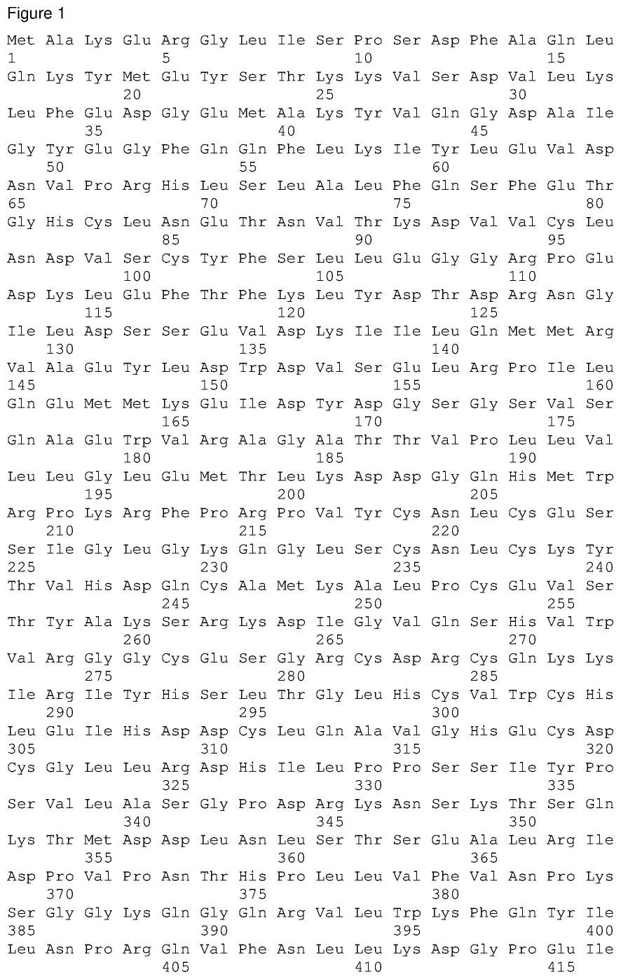

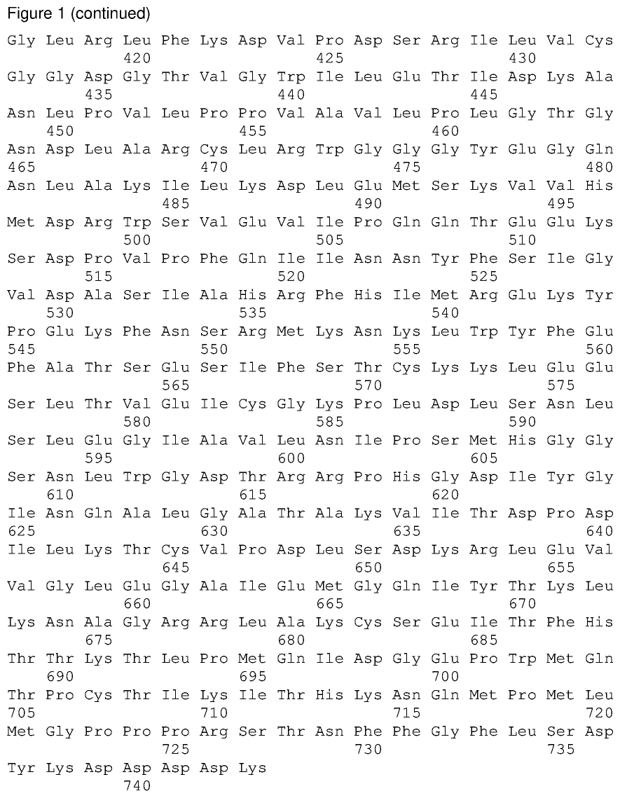

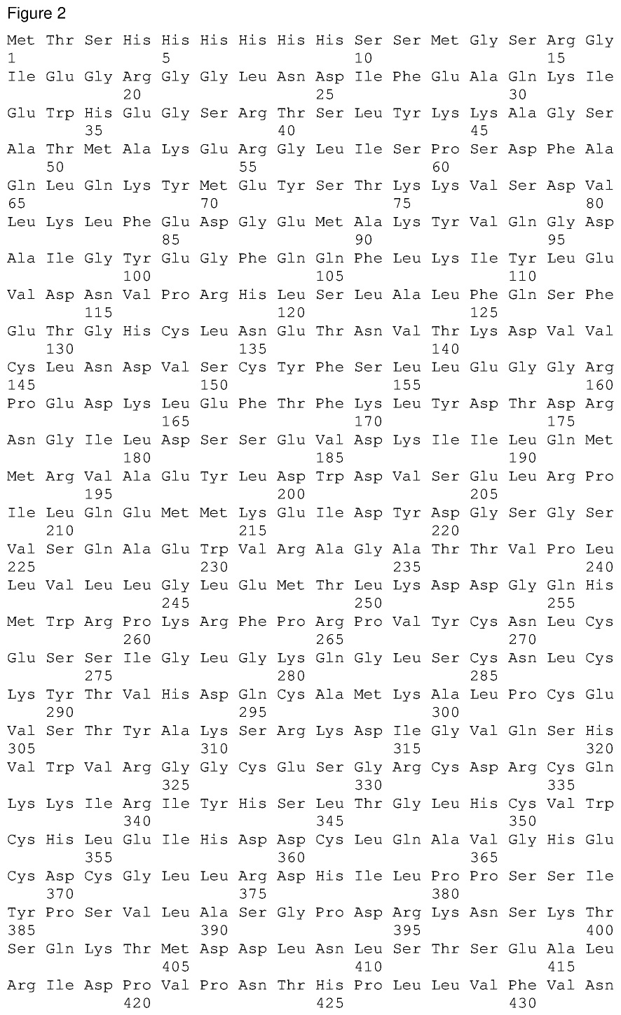

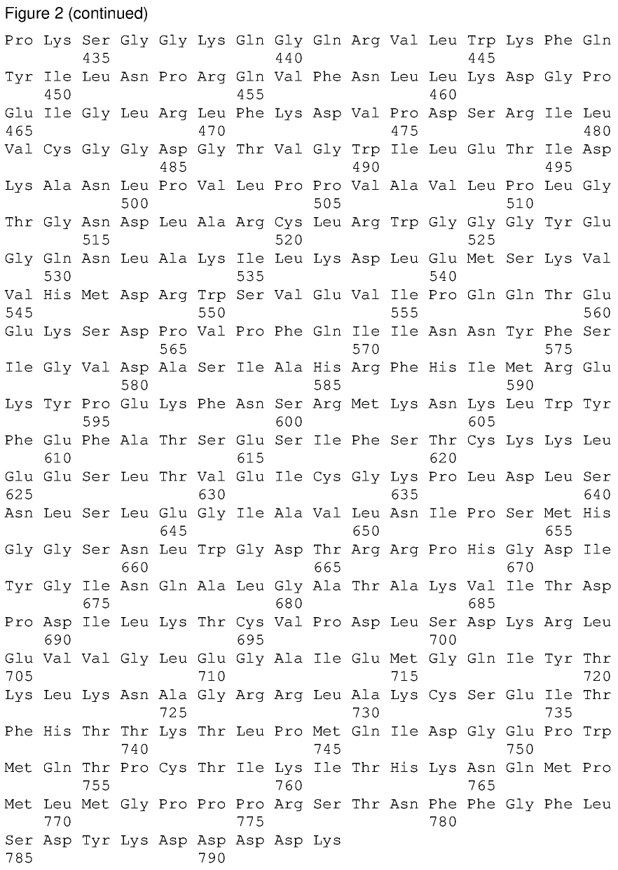

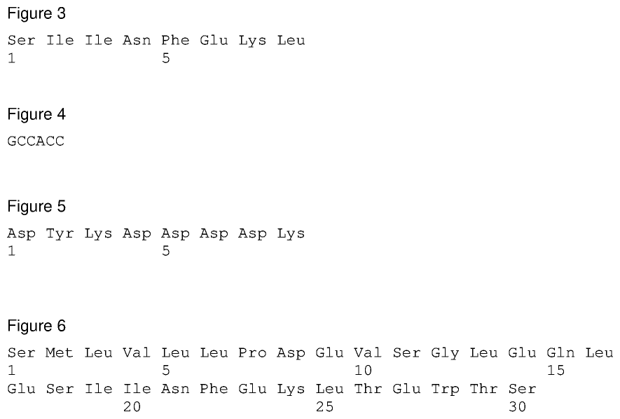

### EXPERIMENTAL SECTION

NMR peak forms are stated as they appear in the spectra, possible higher order effects have not been considered. The multiplicities are stated according to the signal form which appears in the spectrum, NMR-spectroscopic effects of a higher order were not taken into consideration. Multiplicity of the NMR signals: s=singlet, d=doublet, t=triplet, q=quartet, quin=quintet, spt=septed, br=broad signal, m=multiplet. NMR signals: shift in [ppm]. Combinations of multiplicity could be e.g. dd=doublet from doublet.

Chemical names were generated using the ACD/Name software from ACD/Labs. In some cases generally accepted names of commercially available reagents were used in place of ACD/Name generated names.

Table 1 lists the abbreviations used in this paragraph and in the Examples section as far as they are not explained within the text body. Other abbreviations have their meanings customary per se to the skilled person.

TABLE 1
Abbreviations

| | |
|---|---|
| ACN | acetonitrile |
| AcOH | acetic acid |
| CDCl₃ | deuterochloroform |
| BOC | tert-butoxycarbonyl |
| CFSE | carboxyfluorescein succinimidyl ester |
| DAD | diode array detector |
| DEA | diethylamine |
| DMF | N,N-dimethylformamide |
| DMSO-d6 | deuterated dimethyl sulphoxide |
| DMSO | dimethyl sulphoxide |
| ELSD | evaporative light scattering detector |
| ESIpos | electrospray ionization positive |
| Expl. | example |
| HATU | (7-aza-1H-benzotriazol-1-yl)-1,1,3,3- |
| | tetramethyluronium hexafluorophosphate |
| HBTU | O-benzotriazole-N,N,N′,N′-tetramethyluronium |
| | hexafluorophosphate |
| HPLC | high-pressure liquid chromatography |
| LCMS | liquid chromatography coupled with |
| | mass spectrometry |
| LPS | lipopolysaccharide |
| mL | milliliter |
| min. | minute(s) |
| MTBE | methyl tert-butyl ether |
| PBMC | peripheral blood mononuclear cells |
| PyBOP | (benzotriazol-1-yl)oxytripyrrolidinophosphonium |
| | hexafluorophosphate |
| RP-HPLC | reverse-phase high-pressure liquid |
| | chromatography |
| Rt | retention time |
| rt | room temperature |
| sat. | saturated |
| T3P | 2,4,6-tripropyl-1,3,5,2,4,6-trioxatriphosphorinane |
| | 2,4,6-trioxide |
| THF | tetrahydrofurane |
| TFA | trifluoroacetic acid |
| TLC | thin layer chromatography |
| TNFa | tumour necrosis factor alpha |
| μM | micromolar |
| UPLC | Ultra high performance chromatography |

The various aspects of the invention described in this application are illustrated by the following examples which are not meant to limit the invention in any way.

The example testing experiments described herein serve to illustrate the present invention and the invention is not limited to the examples given.

### EXPERIMENTAL SECTION—GENERAL PART

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization.

In some cases, impurities may be stirred out using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

Chromatographic Conditions:

LC-MS (Method 1): Instrument: Waters Acquity UPLCMS SingleQuad; column: Acquity UPLC BEH C18 1.7 μm, 50×2.1 mm; eluent A: water+0.1 vol. % formic acid (99%), eluent B: acetonitrile; gradient: 0-1.6 min. 1-99% B, 1.6-2.0 min. 99% B; flow 0.8 ml/min; temperature: 60° C.; DAD scan: 210-400 nm.

LC-MS (Method 2): Instrument: Waters Acquity UPLCMS SingleQuad; column: Acquity UPLC BEH C18 1.7 μm, 50×2.1 mm; eluent A: water+0.2 vol. % aqueous ammonia (32%), eluent B: acetonitrile; gradient: 0-1.6 min. 1-99% B, 1.6-2.0 min. 99% B; flow 0.8 ml/min; temperature: 60° C.; DAD scan: 210-400 nm.

### EXPERIMENTAL SECTION—INTERMEDIATES

### Intermediate 1

### tert-butyl 2-phenyl-2,8-diazaspiro[4.5]decane-8-carboxylate

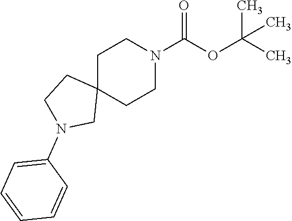 [SMILES: CC(C)(C)OC(=O)N1CCC2(CC1)CCN(c1ccccc1)C2]

A solution of 50 mg tert-butyl 2,8-diazaspiro[4.5]decane-8-carboxylate (208 μmol, CAS 236406-39-6), 24 μL bromobenzene (230 μmol, CAS 108-86-1), 8.18 mg chloro(2-dicyclohexylphosphino-2,4,6-triisopropyl-1,1-biphenyl)[2-(2-amino-1,1-biphenyl)]palladium(II) (10.4 μmol, CAS 1310584-14-5) and 20.0 mg sodium tert-butoxide (208 μmol) in a mixture of 1 mL toluene and of 250 μL tert-butanol was stirred for 17 h at 80° C. The reaction mixture was cooled down to rt, the suspension was filtered, the solid was washed with ethyl acetate and the filtrate was concentrated under reduced pressure. The residue was purified by flash chromatography (silica, heptane/ethyl acetate gradient 0-20%) to give 27 mg of the title compound (26% yield).

¹H NMR (400 MHz, CHLOROFORM-d) δ ppm 1.38-1.43 (m, 9H) 1.47-1.53 (m, 4H) 1.77-1.84 (m, 2H) 3.09 (s, 2H) 3.25-3.34 (m, 4H) 3.37-3.50 (m, 2H) 6.40-6.50 (m, 2H) 6.55-6.65 (m, 1H) 7.12-7.18 (m, 2H).

### Intermediate 2

### 2-phenyl-2,8-diazaspiro[4.5]decane, Salt with Hydrochloric Acid

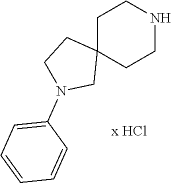 [SMILES: c1ccc(N2CCC3(CCNCC3)C2)cc1]

To 47 mg tert-butyl 2-phenyl-2,8-diazaspiro[4.5]decane-8-carboxylate (149 μmol, intermediate 1) was added 3.0 mL hydrochloric acid (4.0 M in 1,4 dioxane, 12 mmol) and the mixture was stirred for 2 h at rt. The reaction mixture was concentrated under reduced pressure to give 46 mg of the title compound (38% yield).

¹HNMR (400 MHz, CD₃OD) 2.01-2.16 (m, 4H), 2.30 (t, 2H), 3.20-3.36 (m, 4H), 3.73 (s, 3H), 3.89 (t, 2H), 7.41 (t, 1H), 7.54 (t, 2H), 7.56-7.64 (m, 2H).

### Intermediate 3

### tert-butyl 8-(3-cyano-1-methyl-2-oxo-1,2-dihydroquinolin-4-yl)-2,8-diazaspiro[4.5]decane-2-carboxylate

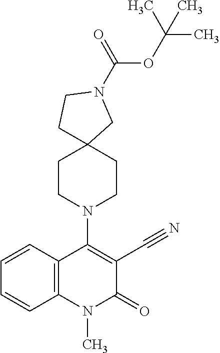 [SMILES: Cn1c(=O)c(C#N)c(N2CCC3(CCN(C(=O)OC(C)(C)C)C3)CC2)c2ccccc21]

678 mg 4-chloro-1-methyl-2-oxo-1,2-dihydroquinoline-3-carbonitrile (2.95 mmol, CAS 150617-68-8, synthesis described in WO2012009649, example 1—compound III), 1.00 g tert-butyl 2,8-diazaspiro[4.5]decane-2-carboxylate (3.54 mmol, CAS 336191-17-4) and 820 μL triethylamine (5.9 mmol) was stirred in 30 mL 2-propanol for 4 h at 90° C. The reaction mixture was cooled down to rt, diluted with ethyl acetate, the organic phase was washed with water and brine, filtered through a water resistant filter and the filtrate was concentrated under reduced pressure. The residue was purified by flash chromatography (silica, dichloromethane/methanol gradient 0-3%) to give 1.15 g of the title compound (95% purity, 88% yield).

¹H-NMR (400 MHz, DMSO-d₆): δ [ppm]=1.29-1.51 (m, 9H), 1.70-1.90 (m, 6H), 2.08 (s, 1H), 3.21 (s, 2H), 3.43-3.84 (m, 7H), 7.32 (t, 1H), 7.51-7.62 (m, 1H), 7.73 (ddd, 1H), 7.82-7.96 (m, 1H).

LC-MS (Method 2): Rₜ=1.24 min; MS (ESIpos): m/z=423.6 [M+H]⁺

### Intermediate 4

### 4-(2,8-diazaspiro[4.5]decan-8-yl)-1-methyl-2-oxo-1,2-dihydroquinoline-3-carbonitrile

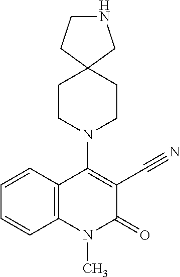 [SMILES: Cn1c(=O)c(C#N)c(N2CCC3(CCNC3)CC2)c2ccccc21]

To a solution of 1.15 g tert-butyl 8-(3-cyano-1-methyl-2-oxo-1,2-dihydroquinolin-4-yl)-2,8-diazaspiro[4.5]decane-2-carboxylate (2.72 mmol, intermediate 3) in 18 mL dichloromethane was added 4.2 mL trifluoroacetic acid (54 mmol) and the mixture was stirred overnight at rt. The reaction mixture was concentrated under reduced pressure and the residue was suspended with toluene and concentrated under reduced pressure to give 1.40 g of the title compound as a TFA salt (85% purity, 136% yield).

¹H NMR (400 MHz, DMSO-d₆) δ ppm 1.77-1.97 (m, 6H) 3.10-3.18 (m, 2H) 3.26-3.36 (m, 2H) 3.51-3.64 (m, 7H) 7.29-7.37 (m, 1H) 7.53-7.62 (m, 1H) 7.70-7.79 (m, 1H) 7.80-7.87 (m, 1H).

LC-MS (Method 2): Rₜ=0.90 min; MS (ESIpos): m/z=323.5 [M+H]⁺

### Intermediate 5

### tert-butyl 2-(3,4-difluorophenyl)-2,8-diazaspiro[4.5]decane-8-carboxylate

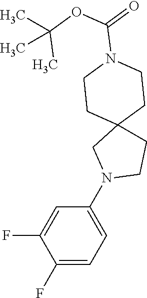 [SMILES: CC(C)(C)OC(=O)N1CCC2(CC1)CCN(c1ccc(F)c(F)c1)C2]

A solution of 100 mg tert-butyl 2,8-diazaspiro[4.5]decane-8-carboxylate (208 μmol, CAS 236406-39-6), 52 μL 4-bromo-1,2-difluorobenzene (460 μmol, CAS 348-61-8), 16.4 mg chloro(2-dicyclohexylphosphino-2,4,6-triisopropyl-1,1-biphenyl)[2-(2-amino-1,1-biphenyl)]palladium(II) (20.8 μmol, CAS 1310584-14-5) and 40.0 mg sodium tert-butoxide (416 μmol) in a mixture of 2 mL toluene and of 500 μL tert-butanol was stirred for 17 h at 80° C. The reaction mixture was cooled down to rt, the suspension was filtered through celite, the solid was washed with ethyl acetate and concentrated under reduced pressure. The residue was purified by flash chromatography (silica, heptane/ethyl acetate gradient 0-15%) to give 47 mg of the title compound (31% yield).

1HNMR (400 MHz, CDCl3) 1.47 (s, 9H), 1.51-1.63 (m, 6H), 3.09 (s, 2H), 3.28-3.41 (m, 4H), 3.44-3.54 (m, 2H), 6.12-6.18 (m, 1H), 6.28 (ddd, 1H), 6.94-7.04 (m, 1H).

### Intermediate 6

### 2-(3,4-difluorophenyl)-2,8-diazaspiro[4.5]decane, Salt with Hydrochloric Acid

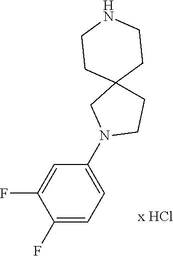 [SMILES: Fc1ccc(N2CCC3(CCNCC3)C2)cc1F]

A solution of 133 mg tert-butyl 2-(3,4-difluorophenyl)-2,8-diazaspiro[4.5]decane-8-carboxylate (377 μmol, intermediate 5) in 3.0 mL hydrochloric acid (4.0 M in 1,4 dioxane, 12 mmol) was stirred for 45 min. at rt. The reaction mixture was concentrated under reduced pressure, the residue was suspended with toluene and concentrated under reduced pressure to give 116 mg of the title compound (103% yield).

¹HNMR (400 MHz, DMSO-d6) 1.72 (t, 4H), 1.88 (t, 2H), 2.97-3.15 (br m, 4H), 3.13 (s, 2H), 3.25 (t, 2H), 6.27 (d, 1H), 6.50 (ddd, 1H), 7.18 (app q, 1H), 8.17 (br s, 1H), 9.07 (br d, 2H).

### Intermediate 7

### tert-butyl 6-(3-cyano-1-methyl-2-oxo-1,2-dihydroquinolin-4-yl)-2,6-diazaspiro[3.4]octane-2-carboxylate

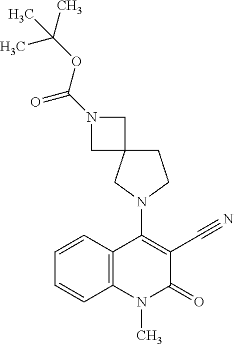 [SMILES: Cn1c(=O)c(C#N)c(N2CCC3(CN(C(=O)OC(C)(C)C)C3)C2)c2ccccc21]

A solution of 3.09 g 4-chloro-1-methyl-2-oxo-1,2-dihydroquinoline-3-carbonitrile (14.1 mmol, CAS 150617-68-8), 3.00 g tert-butyl 2,6-diazaspiro[3.4]octane-2-carboxylate (14.1 mmol, CAS 885270-84-8) and 3.9 mL triethylamine (28 mmol) in 90 mL 2-propanol was stirred for 3 h at 90° C. The reaction mixture was cooled down to rt, diluted with ethyl acetate and water, the suspension was filtered and the solid was dried in vacuum to give 4.70 g of the title compound (95% purity, 80% yield).

¹H NMR (400 MHz, DMSO-d₆) δ ppm 1.35 (s, 9H) 2.11-2.19 (m, 2H) 3.46 (s, 3H) 3.72-3.88 (m, 4H) 3.94-4.03 (m, 2H) 4.09-4.16 (m, 2H) 7.19-7.27 (m, 1H) 7.42-7.47 (m, 1H) 7.62-7.69 (m, 1H) 8.01 (dd, 1H).

LC-MS (Method 2): Rₜ=1.08 min; MS (ESIpos): m/z=395.5 [M+H]⁺

### Intermediate 8

### 4-(2,6-diazaspiro[3.4]octan-6-yl)-1-methyl-2-oxo-1,2-dihydroquinoline-3-carbonitrile, Salt with Trifluoroacetic Acid

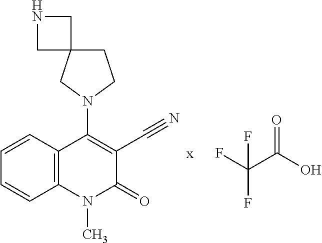 [SMILES: Cn1c(=O)c(C#N)c(N2CCC3(CNC3)C2)c2ccccc21 ; O=C(O)C(F)(F)F]

To a solution of 4.70 g tert-butyl 6-(3-cyano-1-methyl-2-oxo-1,2-dihydroquinolin-4-yl)-2,6-diazaspiro[3.4]octane-2-carboxylate (11.9 mmol, intermediate 7) in 77 mL dichloromethane was added 9.2 mL trifluoroacetic acid (120 mmol) and the mixture was stirred overnight at rt. The reaction mixture was concentrated under reduced pressure and the residue was suspended with toluene and concentrated under reduced pressure (2×) to give 4.80 g of the title compound (137% yield).

¹H-NMR (400 MHz, DMSO-d₆): δ [ppm]=2.25 (t, 2H), 3.88-3.98 (m, 2H), 3.98-4.10 (m, 4H), 4.20 (s, 2H), 7.24 (td, 1H), 7.46 (dd, 1H), 7.55-7.78 (m, 1H), 7.87-8.15 (m, 1H), 8.49-8.83 (m, 1H), 8.88-9.29 (m, 1H).

LC-MS (Method 2): Rₜ=0.72 min; MS (ESIpos): m/z=295.6 [M+H]⁺

### Intermediate 9

### tert-butyl 2-(3-cyano-1-methyl-2-oxo-1,2-dihydroquinolin-4-yl)-2,6-diazaspiro[3.4]octane-6-carboxylate

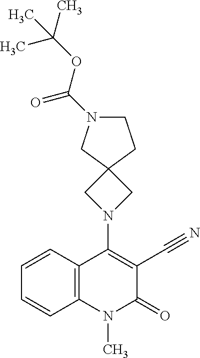 [SMILES: Cn1c(=O)c(C#N)c(N2CC3(CCN(C(=O)OC(C)(C)C)C3)C2)c2ccccc21]

1.03 g 4-chloro-1-methyl-2-oxo-1,2-dihydroquinoline-3-carbonitrile (4.71 mmol, CAS 150617-68-8), 1.50 g tert-butyl 2,6-diazaspiro[3.4]octane-6-carboxylate (7.07 mmol, CAS 885270-86-0) and 1.3 mL triethylamine (9.4 mmol) were stirred in 50 mL 2-propanol for 4 h at 90° C. The reaction mixture was diluted with water and ethyl acetate, the suspension was filtered and the solid was washed with ethyl acetate to give 840 mg of the title compound (99% purity, 45% yield). The filtrate was washed with sodium bicarbonate and brine, filtered through a water resistant filter and concentrated under reduced pressure. The residue was purified by flash chromatography (silica, dichloromethane/methanol gradient 0-3%) to give (solid and purified filtrate together) 1.58 g of the title compound (99% purity, 84% yield).

¹H-NMR (400 MHz, DMSO-d₆): δ [ppm]=1.40 (s, 9H), 2.13 (q, 2H), 3.16-3.31 (m, 2H), 3.42-3.65 (m, 5H), 4.57-4.93 (m, 4H), 7.06-7.26 (m, 1H), 7.47 (dd, 1H), 7.68 (ddd, 1H), 7.79-7.91 (m, 1H).

LC-MS (Method 2): Rₜ=1.10 min; MS (ESIpos): m/z=395.7 [M+H]⁺

### Intermediate 10

### 4-(2,6-diazaspiro[3.4]octan-2-yl)-1-methyl-2-oxo-1,2-dihydroquinoline-3-carbonitrile, Salt with Trifluoroacetic Acid

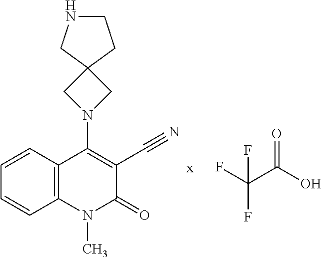 [SMILES: Cn1c(=O)c(C#N)c(N2CC3(CCNC3)C2)c2ccccc21 ; O=C(O)C(F)(F)F]

To 1.23 g tert-butyl 2-(3-cyano-1-methyl-2-oxo-1,2-dihydroquinolin-4-yl)-2,6-diazaspiro[3.4-]octane-6-carboxylate (3.11 mmol, intermediate 9) in 20 mL dichloromethane, was added 2.4 mL trifluoroacetic acid (31 mmol) and the mixture was stirred overnight at rt. The reaction mixture was concentrated under reduced pressure and the residue was suspended with toluene and concentrated under reduced pressure (2×) to give 2.1 g of the title compound (95% purity, 218% yield).

¹H NMR (400 MHz, DMSO-d₆) δ ppm 2.24-2.30 (m, 2H) 3.15-3.29 (m, 2H) 3.45 (t, 2H) 3.49 (s, 3H) 4.69-4.84 (m, 4H) 7.18-7.29 (m, 1H) 7.45-7.55 (m, 1H) 7.63-7.84 (m, 2H).

LC-MS (Method 2): Rₜ=0.77 min; MS (ESIpos): m/z=295.7 [M+H]⁺

### Intermediate 11

### tert-butyl 2-(3-methoxyphenyl)-2,8-diazaspiro[4.5]decane-8-carboxylate

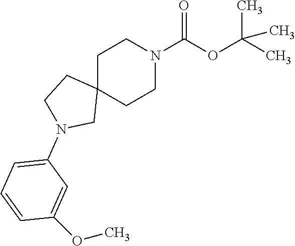 [SMILES: COc1cccc(N2CCC3(CCN(C(=O)OC(C)(C)C)CC3)C2)c1]

A mixture of 250 mg tert-butyl 2,8-diazaspiro[4.5]decane-8-carboxylate (1.04 mmol, CAS 236406-39-6), 140 μL 1-bromo-3-methoxybenzene (1.1 mmol, CAS 2398-37-0), 11.7 mg palladium(II)acetate (52 μmol), 24.8 mg dicyclohexyl[2′,4′,6′-tri(propan-2-yl)biphenyl-2-yl]phosphane (52 μmol, CAS 564483-18-7) and 100 mg sodium tert-butoxide (1.04 mmol) in a mixture of 3 mL toluene and of 600 μL tert-butanol was heated at reflux overnight. The reaction mixture was filtered through celite, washed with ethyl acetate and the filtrate was concentrated under reduced pressure. The residue was purified by flash chromatography (silica, heptane/ethyl acetate gradient 5-20%) to give 194 mg of the title compound (54% yield).

1HNMR (400 MHz, CDCl3) 1.47 (s, 9H), 1.53-1.62 (m, 4H), 1.87 (t, 2H), 3.15 (s, 2H), 3.31-3.40 (m, 4H), 3.46-3.54 (m, 2H), 3.80 (s, 3H), 6.09 (t, 1H), 6.17 (dd, 1H), 6.25 (dd, 1H), 7.13 (t, 1H).

### Intermediate 12

### 2-(3-methoxyphenyl)-2,8-diazaspiro[4.5]decane, Salt with Hydrochloric Acid

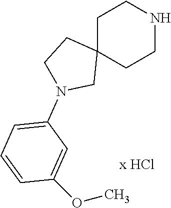 [SMILES: COc1cccc(N2CCC3(CCNCC3)C2)c1]

To 194 mg tert-butyl 2-(3-methoxyphenyl)-2,8-diazaspiro[4.5]decane-8-carboxylate (560 μmol, intermediate 11) in 1.0 mL methanol was added 1.9 mL hydrochloric acid (4.0 M in 1,4 dioxane, 5.6 mmol) and the mixture was stirred for 3 h at rt. The reaction mixture was concentrated under reduced pressure to give 164 mg of the title compound (103% yield).

1HNMR (300 MHz, DMSO-d6) 1.74 (t, 4H), 1.89 (t, 2H), 3.01-3.12 (br m, 4H), 3.16 (s, 2H), 3.29 (t, 2H), 3.71 (s, 3H), 6.08-6.13 (br m, 1H), 6.15-6.24 (m, 2H), 6.72 (br s, 1H), 7.05 (t, 1H), 9.03 (br s, 1H).

### Intermediate 13

### tert-butyl 2-(2-methoxyphenyl)-2,8-diazaspiro[4.5]decane-8-carboxylate

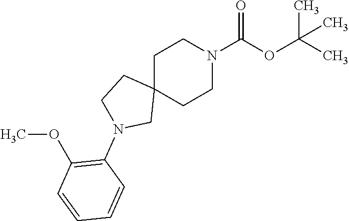 [SMILES: COc1ccccc1N1CCC2(CCN(C(=O)OC(C)(C)C)CC2)C1]

A mixture of 250 mg tert-butyl 2,8-diazaspiro[4.5]decane-8-carboxylate (1.04 mmol, CAS 236406-39-6), 140 μL 1-bromo-2-methoxybenzene (1.1 mmol, CAS 578-57-4), 11.7 mg palladium(II)acetate (52 μmol), 24.8 mg dicyclohexyl[2′,4′,6′-tri(propan-2-yl)biphenyl-2-yl]phosphane (52.0 μmol, CAS 564483-18-7) and 100 mg sodium tert-butoxide (1.04 mmol) in a mixture of 5 mL toluene and of 1.3 mL tert-butanol was heated at reflux overnight. The reaction mixture was filtered through celite, washed with ethyl acetate and the filtrate was concentrated under reduced pressure. The residue was purified by flash chromatography (silica, heptane/ethyl acetate gradient 5-25%) to give 32 mg of the title compound (9% yield).

¹HNMR (400 MHz, CDCl₃) 1.47 (s, 9H), 1.54-1.64 (m, 4H), 1.79 (t, 2H), 3.20 (s, 2H), 3.33-3.50 (m, 6H), 3.82 (s, 3H), 6.72 (dd, 1H), 6.77-6.91 (m, 3H).

### Intermediate 14

### 2-(2-methoxyphenyl)-2,8-diazaspiro[4.5]decane, Salt with Hydrochloric Acid

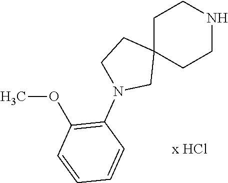 [SMILES: COc1ccccc1N1CCC2(CCNCC2)C1]

To 46 mg tert-butyl 2-(2-methoxyphenyl)-2,8-diazaspiro[4.5]decane-8-carboxylate (133 μmol, intermediate 13) was added 1.0 mL hydrochloric acid (4.0 M in 1,4 dioxane, 4.0 mmol) and the mixture was stirred overnight at rt. The reaction mixture was concentrated under reduced pressure to give 38 mg of the title compound (96% yield).

¹HNMR (400 MHz, DMSO-d6) 1.84 (br s, 4H), 2.02 (br s, 2H), 3.02-3.14 (br m, 6H), 3.53-3.67 (br m, 2H), 3.86 (br s, 3H), 6.89-7.01 (br m, 2H), 7.03-7.22 (br m, 2H).

### Intermediate 15

### tert-butyl 2-[3-(dimethylamino)phenyl]-2,8-diazaspiro[4.5]decane-8-carboxylate

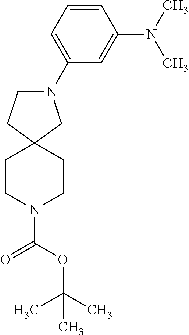 [SMILES: CN(C)c1cccc(N2CCC3(CCN(C(=O)OC(C)(C)C)CC3)C2)c1]

A mixture of 250 mg tert-butyl 2,8-diazaspiro[4.5]decane-8-carboxylate (1.04 mmol, CAS 236406-39-6), 180 μL 3-bromo-N,N-dimethylaniline (1.2 mmol, CAS 16518-62-0), 164 mg chloro(2-dicyclohexylphosphino-2,4,6-triisopropyl-1,1-biphenyl)[2-(2-amino-1,1-biphenyl)]palladium(II) (208 μmol, CAS 1310584-14-5) and 100 mg sodium tert-butoxide (1.04 mmol) in a mixture of 15 mL toluene and of 5 mL tert-butanol was heated at reflux for 18 h. The reaction mixture was filtered through celite, washed with ethyl acetate and the filtrate was concentrated under reduced pressure. The residue was purified by flash chromatography (silica, heptane/ethyl acetate gradient 0-80%) to give 170 mg of the title compound (44% yield).

¹HNMR (CDCl₃, 400 MHz): 1.49 (s, 9H); 1.51-1.66 (m, 4H); 1.88 (t, 2H); 2.95 (s, 6H); 3.19 (s, 2H); 3.34-3.43 (m, 4H); 3.47-3.56 (m, 2H); 5.90 (t, 1H); 6.01 (dd, 1H); 6.16 (dd, 1H); 7.11 (t, 1H).

### Intermediate 16

### 3-(2,8-diazaspiro[4.5]decan-2-yl)-N,N-dimethylaniline, Salt with Hydrochloric Acid

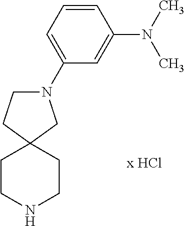 [SMILES: CN(C)c1cccc(N2CCC3(CCNCC3)C2)c1]

To 170 mg tert-butyl 2-[3-(dimethylamino)phenyl]-2,8-diazaspiro[4.5]decane-8-carboxylate (473 μmol, intermediate 15) in 5.0 mL 1,4-dioxane was added 25 mL hydrochloric acid (4.0 M in 1,4 dioxane, 100 mmol) and the mixture was stirred for 18 h at rt. The mixture was concentrated (to the half amount) under reduced pressure. The suspension was filtered and the solid was dried in vacuum to give 110 mg of the title compound (70% yield).

¹HNMR (DMSO-d6, 400 MHz): 1.72-1.82 (m, 4H); 1.97 (t, 2H); 3.12 (s, 6H); 3.24 (s, 2H); 3.37 (t, 2H); 3.60 (s, 4H); 6.66 (d, 1H); 6.97 (s, 1H); 7.00 (d, 1H); 7.33 (t, 1H); 9.10 (br s, 1H); 9.23 (br s, 1H).

### Intermediate 17

### tert-butyl 2-(2-nitrophenyl)-2,8-diazaspiro[4.5]decane-8-carboxylate

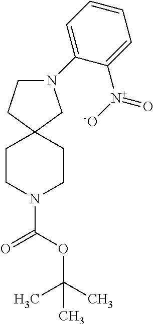

110 μL 1-fluoro-2-nitrobenzene (1.0 mmol, CAS 1493-27-2), 250 mg tert-butyl 2,8-diazaspiro[4.5]decane-8-carboxylate (1.04 mmol, CAS 236406-39-6) and 910 μL N,N-diisopropylethylamine (5.2 mmol) was stirred in 25 mL dimethyl sulfoxide for 18 h at 100° C. The reaction mixture was diluted with water and extracted with ethyl acetate (3×). The combined organic phases were washed with water and brine, dried over magnesium sulfate, filtered and the filtrate was concentrated under reduced pressure. The residue was purified by flash chromatography (silica, heptane/ethyl acetate gradient 0-50%) to give 225 mg of the title compound (92% purity, 55% yield).

¹HNMR (CDCl₃, 400 MHz): 1.46 (s, 9H); 1.56 (t, 4H); 1.89 (t, 2H); 3.06 (s, 2H); 3.29-2.49 (m, 6H); 6.76 (t, 1H); 6.89 (d, 1H); 7.35-7.40 (m, 1H); 7.74 (dd, 1H).

### Intermediate 18

### tert-butyl 2-[2-(dimethylamino)phenyl]-2,8-diazaspiro[4.5]decane-8-carboxylate

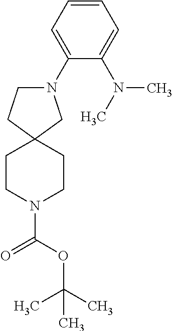 [SMILES: CN(C)c1ccccc1N1CCC2(CCN(C(=O)OC(C)(C)C)CC2)C1]

To 203 mg tert-butyl 2-(2-nitrophenyl)-2,8-diazaspiro[4.5]decane-8-carboxylate (560 μmol, intermediate 17) in 9 mL ethanol was added 200 μL formaldehyde (37% purity in water, 2.7 mmol) and 7.45 mg palladium on carbon (50-70% wetted powder, 70.0 μmol). The suspension was stirred under hydrogen atmosphere for 18 h at rt. The reaction mixture was filtered through celite, washed with ethyl acetate and concentrated under reduced pressure. The residue was dissolved in dichloromethane. The organic phase was washed with brine and dried over magnesium sulfate, filtered and concentrated under reduced pressure. The residue was purified by flash chromatography (silica, heptane/ethyl acetate gradient 0-50%) to give 83 mg of the title compound (97% purity, 40% yield).

¹HNMR (400 MHz, CDCl₃): 1.49 (s, 9H); 1.56-1.69 (m, 4H); 1.78 (t, 2H); 2.70 (s, 6H); 3.13 (s, 2H); 3.32 (t, 2H); 3.42-3.48 (m, 4H); 6.81 (dd, 1H); 6.86 (td, 1H); 6.93 (td, 1H); 6.98 (dd, 1H).

### Intermediate 19

### 2-(2,8-diazaspiro[4.5]decan-2-yl)-N,N-dimethylaniline, Salt with Hydrochloric Acid

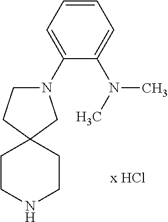 [SMILES: CN(C)c1ccccc1N1CCC2(CCNCC2)C1]

To 83 mg tert-butyl 2-[2-(dimethylamino)phenyl]-2,8-diazaspiro[4.5]decane-8-carboxylate (231 μmol, intermediate 18) was added 12 mL hydrochloric acid (4.0 M in 1,4 dioxane, 100 mmol) and the mixture was stirred for 72 h at rt. The reaction mixture was filtered and the solid was washed with triethylamine and acetonitrile. The filtrate was washed with an aqueous solution of sodium bicarbonate and the mixture was extracted with ethyl acetate (3×). The combined organic phases were washed with brine, dried over magnesium sulfate, filtered and concentrated under reduced pressure to give 129 mg of the title compound (215% yield).

¹H NMR (400 MHz, CDCl3) δ ppm 1.17-1.42 (m, 2H), 1.73-1.83 (m, 2H), 1.84-2.05 (m, 4H), 2.55-2.73 (s, 6H), 3.04-3.34 (m, 8H), 6.67-6.78 (m, 1H), 6.79-6.98 (m, 3H), 9.32-9.65 (bs, 2H).

### Intermediate 20

### tert-butyl 2-(4-methoxyphenyl)-2,8-diazaspiro[4.5]decane-8-carboxylate

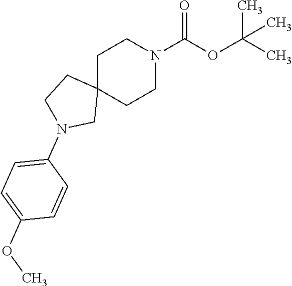 [SMILES: COc1ccc(N2CCC3(CCN(C(=O)OC(C)(C)C)CC3)C2)cc1]

A mixture of 250 mg tert-butyl 2,8-diazaspiro[4.5]decane-8-carboxylate (1.04 mmol, CAS 236406-39-6), 140 μL 1-bromo-4-methoxybenzene (1.1 mmol, CAS 104-92-7), 11.7 mg palladium(II)acetate (52 μmol), 24.8 mg dicyclohexyl[2′,4′,6′-tri(propan-2-yl)biphenyl-2-yl]phosphane (52.0 μmol, CAS 564483-18-7) and 100 mg sodium tert-butoxide (1.04 mmol) in 5 mL toluene and 1.3 mL tert-butanol was heated at reflux overnight. The reaction mixture was filtered through celite, washed with ethyl acetate and the filtrate was concentrated under reduced pressure. The residue was purified by flash chromatography (silica, heptane/ethyl acetate gradient 10-50%) to give 113 mg of the title compound (98% purity, 31% yield).

¹HNMR (400 MHz, CDCl₃) 1.46 (s, 9H), 1.50-1.60 (m, 4H), 1.86 (t, 2H), 3.11 (s, 2H), 3.27-3.40 (m, 4H), 3.43-3.53 (m, 2H), 3.75 (s, 3H), 6.50 (d, 2H), 6.84 (d, 2H).

### Intermediate 21

### 2-(4-methoxyphenyl)-2,8-diazaspiro[4.5]decane, Salt with Hydrochloric Acid

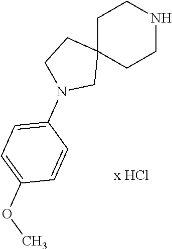 [SMILES: COc1ccc(N2CCC3(CCNCC3)C2)cc1]

A solution of 113 mg tert-butyl 2-(4-methoxyphenyl)-2,8-diazaspiro[4.5]decane-8-carboxylate (326 μmol, intermediate 20) in 2.0 mL hydrochloric acid (4.0 M in 1,4 dioxane, 8.0 mmol) was stirred for 1 h at rt. The reaction mixture was concentrated under reduced pressure to give 94 mg of the title compound (82% purity, 83% yield).

¹HNMR (400 MHz, CD₃OD) 2.02-2.20 (m, 4H), 2.33 (t, 2H), 3.23-3.34 (m, 4H), 3.71-3.99 (m, 7H), 7.11 (d, 2H), 7.71 (d, 2H).

### Intermediate 22

### tert-butyl 2-(3-cyano-1-methyl-2-oxo-1,2-dihydroquinolin-4-yl)-2,6-diazaspiro[3.5]nonane-6-carboxylate

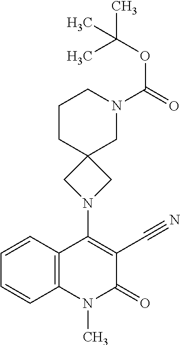 [SMILES: Cn1c(=O)c(C#N)c(N2CC3(CCCN(C(=O)OC(C)(C)C)C3)C2)c2ccccc21]

A solution of 322 mg 4-chloro-1-methyl-2-oxo-1,2-dihydroquinoline-3-carbonitrile (1.47 mmol, CAS 150617-68-8), 1.0 g tert-butyl 2,6-diazaspiro[3.5]nonane-6-carboxylate (4.42 mmol, CAS 885272-17-3) and 620 μL triethylamine (4.4 mmol) in 7.5 mL 2-propanol was heated for 4 h at 90° C. The reaction mixture was diluted with ethyl acetate, the organic phase was washed with water and brine, filtered through a water resistant filter and the filtrate was concentrated under reduced pressure. The residue was purified by flash chromatography (silica, dichloromethane/methanol gradient 0-3%) to give 275 mg of the title compound (95% purity, 43% yield).

¹H NMR (400 MHz, DMSO-d₆) δ ppm 1.38 (s, 9H) 1.41-1.52 (m, 2H) 1.78-1.87 (m, 2H) 3.22-3.29 (m, 2H) 3.46-3.57 (m, 5H) 4.36-4.53 (m, 4H) 7.17-7.27 (m, 1H) 7.43-7.53 (m, 1H) 7.63-7.76 (m, 1H) 7.79-7.94 (m, 1H).

LC-MS (Method 2): Rₜ=1.15 min; MS (ESIpos): m/z=409.7 [M+H]⁺

### Intermediate 23

### 4-(2,6-diazaspiro[3.5]nonan-2-yl)-1-methyl-2-oxo-1,2-dihydroquinoline-3-carbonitrile, Salt with Trifluoroacetic Acid

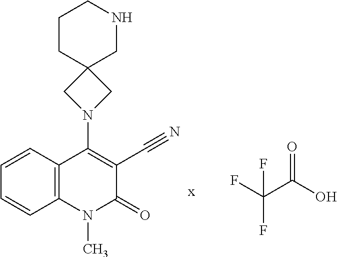 [SMILES: Cn1c(=O)c(C#N)c(N2CC3(CCCNC3)C2)c2ccccc21 ; O=C(O)C(F)(F)F]

To a solution of 270 mg tert-butyl 2-(3-cyano-1-methyl-2-oxo-1,2-dihydroquinolin-4-yl)-2,6-diazaspiro[3.5]nonane-6-carboxylate (661 μmol, intermediate 22) in 4.3 mL dichloromethane was added 510 μL trifluoroacetic acid (6.6 mmol) and the mixture was stirred for 4 h at rt. The mixture was concentrated under reduced pressure and the residue was diluted with toluene (2×).

The solvent was evaporated to give 290 mg of the title compound (95% purity, 135% yield).

¹H NMR (400 MHz, DMSO-d₆) δ ppm 1.64-1.75 (m, 2H) 1.85-1.95 (m, 2H) 2.90-3.04 (m, 2H) 3.30-3.35 (m, 2H) 3.46-3.51 (m, 3H) 4.47-4.67 (m, 4H) 7.21-7.28 (m, 2H) 7.42-7.53 (m, 1H) 7.60-7.74 (m, 1H) 7.75-7.90 (m, 1H).

LC-MS (Method 2): Rₜ=0.82 min; MS (ESIpos): m/z=309.8 [M+H]⁺

### Intermediate 24

### tert-butyl 7-(3-cyano-1-methyl-2-oxo-1,2-dihydroquinolin-4-yl)-2,7-diazaspiro[4.4]nonane-2-carboxylate

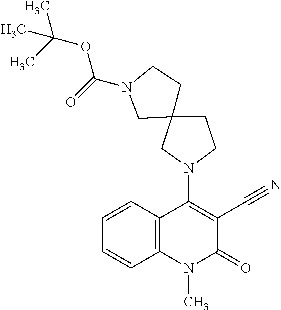 [SMILES: Cn1c(=O)c(C#N)c(N2CCC3(CCN(C(=O)OC(C)(C)C)C3)C2)c2ccccc21]

A solution of 500 mg 4-chloro-1-methyl-2-oxo-1,2-dihydroquinoline-3-carbonitrile (2.29 mmol, CAS 150617-68-8), 776 mg tert-butyl 2,7-diazaspiro[4.4]nonane-2-carboxylate (3.43 mmol, CAS 236406-49-8) and 640 μL triethylamine (4.6 mmol) in 20 mL 2-propanol was heated for 4 h at 90° C. The reaction mixture was diluted with ethyl acetate, the organic phase was washed with water and brine, filtered through a water resistant filter and the filtrate was concentrated under reduced pressure. The residue was purified by flash chromatography (silica, dichloromethane/methanol gradient 0-3%) to give 750 mg of the title compound (98% purity, 79% yield).

¹H NMR (400 MHz, DMSO-d₆) δ ppm 1.33-1.41 (m, 9H) 1.79-2.03 (m, 4H) 3.21-3.27 (m, 2H) 3.46-3.48 (m, 5H) 3.86-3.99 (m, 2H) 4.01-4.14 (m, 2H) 7.19-7.26 (m, 1H) 7.42-7.48 (m, 1H) 7.62-7.69 (m, 1H) 8.03-8.08 (m, 1H).

LC-MS (Method 2): Rₜ=1.11 min; MS (ESIpos): m/z=409.0 [M+H]⁺

### Intermediate 25

### 4-(2,7-diazaspiro[4.4]nonan-2-yl)-1-methyl-2-oxo-1,2-dihydroquinoline-3-carbonitrile, Salt with Trifluoroacetic Acid

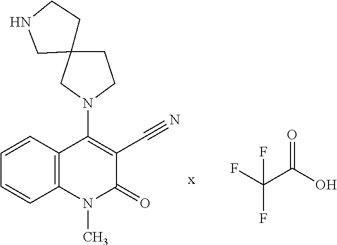 [SMILES: Cn1c(=O)c(C#N)c(N2CCC3(CCNC3)C2)c2ccccc21 ; O=C(O)C(F)(F)F]

To a solution of 635 mg tert-butyl 7-(3-cyano-1-methyl-2-oxo-1,2-dihydroquinolin-4-yl)-2,7-diazaspiro[4.4]nonane-2-carboxylate (1.55 mmol, intermediate 24) in 10 mL dichloromethane was added 2.2 mL trifluoroacetic acid (29 mmol) and the mixture was stirred for 24 h at rt. The mixture was concentrated under reduced pressure and the residue was diluted with toluene (2×). The solvent was evaporated to give 980 mg of the title compound (85% purity, 174% yield).

¹H NMR (400 MHz, DMSO-d₆) δ ppm 1.88-2.15 (m, 4H) 3.12-3.38 (m, 4H) 3.48 (s, 3H) 3.92-4.16 (m, 4H) 7.20-7.29 (m, 1H) 7.43-7.49 (m, 1H) 7.62-7.74 (m, 1H) 7.95-8.08 (m, 1H).

LC-MS (Method 2): Rₜ=0.80 min; MS (ESIpos): m/z=309.8 [M+H]⁺

### Intermediate 26

### 7-bromo-1-methyl-2H-3,1-benzoxazine-2,4(1H)-dione

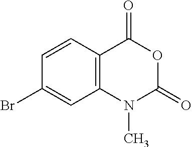

To a solution of 50 g 7-bromo-2H-3,1-benzoxazine-2,4(1H)-dione (207 mmol, CAS 76561-16-5) and 72 mL N,N-diisopropylethylamine (413 mmol) in 400 mL dimethylacetamide was added 39 mL iodomethane (620 mmol, CAS 74-88-4) at rt and the mixture was stirred overnight. The reaction was cooled to 0° C. and 200 m L water was slowly added. The solid that precipitated from this procedure was collected by filtration, washed with water and dried in an oven at 50° C. 48.1 g of the title compound were obtained (91% yield).

¹H-NMR (400 MHz, DMSO-d6) δ[ppm]: 3.46 (s, 3H); 7.52 (dd, 1H); 7.70 (d, 1H); 7.90 (d, 1H).

LC-MS (Method 1): Rₜ=0.92 min; MS (ESIpos): m/z=256.1 [M+H]⁺

### Intermediate 27

### 7-bromo-4-hydroxy-1-methyl-2-oxo-1,2-dihydroquinoline-3-carbonitrile

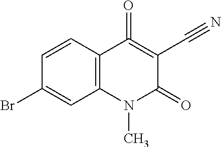

A solution of 40 g 7-bromo-1-methyl-2H-3,1-benzoxazine-2,4(1H)-dione (156 mmol, intermediate 26) in 320 mL THF was slowly treated with 175 mL triethylamine (1.2 mol) followed by the addition of 25 mL ethyl cyanoacetate (234 mmol, CAS 105-56-6) at rt. The reaction was heated at 60° C. and stirred at that temperature over night. Further 25 mL ethyl cyanoacetate (234 mmol, CAS 105-56-6) were added and the reaction was stirred at 70° C. for further 5 h. After cooling to rt, water was added and THF was evaporated in vacuum. The mixture was acidified to pH=1 by addition of hydrochloric acid (2 M) and extracted with ethyl acetate (3×). The combined organic layers were evaporated in vacuum and the residue was stirred first with hexane, decanted and then stirred with a small amount ethyl acetate/hexane. The residue was filtered and 46 g of the title material were obtained in two crops (106% yield).

¹H-NMR (400 MHz, DMSO-d6) δ[ppm]: 3.51 (s, 3H); 6.67 (bs, 1H); 7.46 (dd, 1H); 7.71 (d, 1H); 7.96 (d, 1H).

LC-MS (Method 1): Rₜ=0.75 min; MS (ESIpos): m/z=279.1 [M+H]⁺

### Intermediate 28

### 7-bromo-4-chloro-1-methyl-2-oxo-1,2-dihydroquinoline-3-carbonitrile

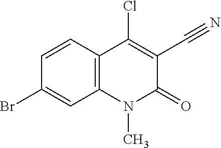 [SMILES: Cn1c(=O)c(C#N)c(Cl)c2ccc(Br)cc21]

A mixture of 16 g 7-bromo-4-hydroxy-1-methyl-2-oxo-1,2-dihydroquinoline-3-carbonitrile (57 mmol, intermediate 27) and 100 mL phosphorus trichloride (1.05 mol, CAS 7719-12-2) was stirred at 90° C. overnight. After cooling to rt, hexane was added and the reaction was filtered. The solid was washed with sat. sodium bicarbonate solution and water. The obtained residue was dried in an oven at 50° C. overnight to give 13.2 g of the title compound (77% yield).

¹H-NMR (400 MHz, DMSO-d6) δ[ppm]: 3.64 (s, 3H); 7.66 (dd, 1H); 7.94-7.98 (m, 2H).

LC-MS (Method 1): Rₜ=1.11 min; MS (ESIpos): m/z=297.2 [M+H]⁺

### Intermediate 29

### tert-butyl 6-(7-bromo-3-cyano-1-methyl-2-oxo-1,2-dihydroquinolin-4-yl)-2,6-diazaspiro-[3.4]octane-2-carboxylate

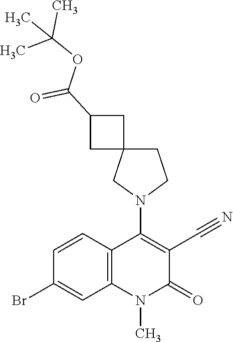 [SMILES: Cn1c(=O)c(C#N)c(N2CCC3(CC(C(=O)OC(C)(C)C)C3)C2)c2ccc(Br)cc21]

A solution of 2.8 g 7-bromo-4-chloro-1-methyl-2-oxo-1,2-dihydroquinoline-3-carbonitrile (9.42 mmol, intermediate 28), 2.00 g tert-butyl 2,6-diazaspiro[3.4]octane-2-carboxylate (9.42 mmol, CAS 885270-84-8) and 2.6 mL triethylamine (19 mmol) in 60 mL 2-propanol was stirred for 3 h at 90° C. After this time, water and ethyl acetate w ere added. The precipitate that was generated by this procedure was collected by filtration and dried in vacuum. 1.15 g of the title compound were obtained (98% purity, 25% yield).

¹H NMR (400 MHz, DMSO-d₆) δ ppm 1.33-1.40 (m, 9H) 2.12-2.19 (m, 2H) 3.44 (s, 3H) 3.72-3.91 (m, 4H) 3.94-4.02 (m, 2H) 4.09-4.16 (m, 2H) 7.35-7.41 (m, 1H) 7.61-7.66 (m, 1H) 7.90-7.97 (m, 1H).

LC-MS (Method 2): Rₜ=1.20 min; MS (ESIpos): m/z=475.4 [M+H]⁺

### Intermediate 30

### 7-bromo-4-(2,6-diazaspiro[3.4]octan-6-yl)-1-methyl-2-oxo-1,2-dihydroquinoline-3-carbonitrile, Salt with Trifluoroacetic Acid

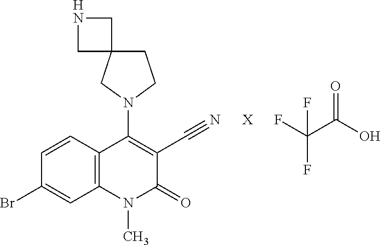

To a solution of 930 mg tert-butyl 6-(7-bromo-3-cyano-1-methyl-2-oxo-1,2-dihydroquinolin-4-yl)-2,6-diazaspiro[3.4]octane-2-carboxylate (1.96 mmol, intermediate 29) in 30 mL dichloromethane was added 3 mL trifluoroacetic acid (39 mmol) and the mixture was stirred for 72 h at rt. The mixture was concentrated under reduced pressure and the residue was diluted with toluene (3×). The solvent was evaporated to give 1.2 g of the title compound (90% purity, 147% yield).

¹H NMR (400 MHz, DMSO-d) δ ppm 2.18-2.27 (m, 2H), 3.47 (s, 3H), 3.88-4.10 (m, 8H), 7.38-7.44 (m, 1H), 7.65-7.69 (m, 1H), 7.85-7.94 (m, 1H), 8.43-8.56 (bs, 1H).

LC-MS (Method 2): Rₜ=0.88 min; MS (ESIpos): m/z=373.4 [M+H]⁺

### Intermediate 31

### tert-butyl 8-(7-bromo-3-cyano-1-methyl-2-oxo-1,2-dihydroquinolin-4-yl)-2,8-diazaspiro-[4.5]decane-2-carboxylate

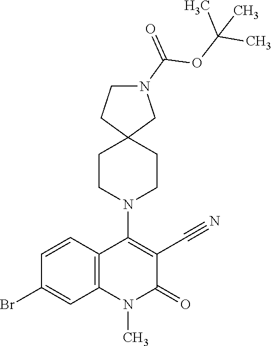 [SMILES: Cn1c(=O)c(C#N)c(N2CCC3(CCN(C(=O)OC(C)(C)C)C3)CC2)c2ccc(Br)cc21]

A suspension of 2.6 g 7-bromo-4-chloro-1-methyl-2-oxo-1,2-dihydroquinoline-3-carbonitrile (8.74 mmol, intermediate 28), 2.10 g tert-butyl 2,8-diazaspiro[4.5]decane-2-carboxylate (8.74 mmol, CAS 336191-17-4) and 2.4 mL triethylamine (17 mmol) in 20 mL 2-propanol was stirred for 2 h at 90° C. After this time, water and ethyl acetate were added. The precipitate that was generated by this procedure was collected by filtration and dried in vacuum. 3.2 g of the title compound were obtained (95% purity, 69% yield).

¹H NMR (400 MHz, DMSO-d₆) δ ppm 1.34-1.49 (m, 9H) 1.67-1.88 (m, 6H) 3.16-3.25 (m, 2H) 3.45-3.65 (m, 7H) 7.47 (d, 1H) 7.72-7.87 (m, 2H).

LC-MS (Method 2): Rₜ=1.40 min; MS (ESIpos): m/z=501.3 [M+H]⁺

### Intermediate 32

### tert-butyl 8-[3-cyano-1-methyl-7-(4-methylpiperazin-1-yl)-2-oxo-1,2-dihydroquinolin-4-yl]-2,8-diazaspiro[4.5]decane-2-carboxylate

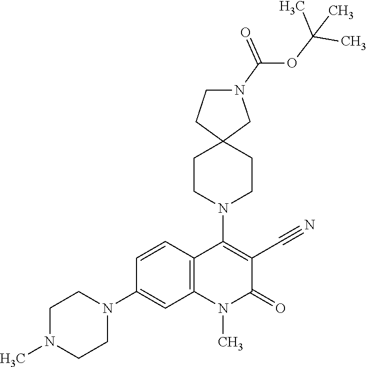 [SMILES: CN1CCN(c2ccc3c(N4CCC5(CCN(C(=O)OC(C)(C)C)C5)CC4)c(C#N)c(=O)n(C)c3c2)CC1]

To a stirred solution of 500 mg tert-butyl 8-(7-bromo-3-cyano-1-methyl-2-oxo-1,2-dihydroquinolin-4-yl)-2,8-diazaspiro[4.5]decane-2-carboxylate (997 μmol, intermediate 31) in 10 mL 1,4-dioxane were added 270 μL 1-methylpiperazine (2.4 mmol, CAS 109-01-3), 1.3 g cesium carbonate (3.99 mmol) and 157 mg chloro(2-dicyclohexylphosphino-2,4,6-triisopropyl-1,1-biphenyl)[2-(2-amino-1,1-biphenyl)]palladium(II) (199 μmol, CAS 1310584-14-5). The mixture was stirred for 2 h at 110° C. and 72 h at rt. The re action mixture was diluted with water and the aqueous phase was extracted with ethyl acetate. The combined organic phases were washed with water and brine, filtered (using a water resistant filter) and concentrated under reduced pressure. The residue was purified by flash chromatography (silica, dichloromethane/methanol gradient 0-3%) to give 130 mg of the title compound (98% purity, 25% yield).

¹H NMR (400 MHz, DMSO-d₆) δ ppm 1.41 (s, 9H) 1.61-1.88 (m, 6H) 2.3 (s, 3H) 2.42-2.47 (m, 4H) 3.15-3.24 (m, 2H) 3.39-3.59 (m, 11H) 6.60-6.70 (m, 1H) 6.91-7.00 (m, 1H) 7.59-7.71 (m, 1H).

LC-MS (Method 2): Rₜ=1.22 min; MS (ESIpos): m/z=521.5 [M+H]⁺

### Intermediate 33

### 4-(2,8-diazaspiro[4.5]decan-8-yl)-1-methyl-7-(4-methylpiperazin-1-yl)-2-oxo-1,2-dihydro-quinoline-3-carbonitrile, Salt with Hydrochloric Acid

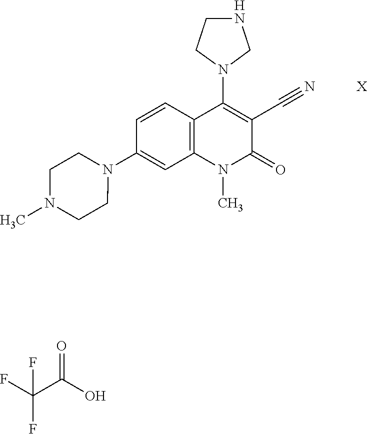

To a solution of 130 mg tert-butyl 8-[3-cyano-1-methyl-7-(4-methylpiperazin-1-yl)-2-oxo-1,2-dihydroquinolin-4-yl]-2,8-diazaspiro[4.5]decane-2-carboxylate (250 μmol, intermediate 32) in 3 mL dichloromethane was added 380 μL trifluoroacetic acid (5 mmol) and the mixture was stirred overnight at rt. The mixture was concentrated under reduced pressure and the residue was diluted with toluene (2×). The solvent was evaporated to give 105 mg of the title compound (90% purity, 90% yield).

¹H NMR (400 MHz, DMSO-d) δ ppm 1.72-1.87 (m, 4H), 1.88-1.96 (m, 2H), 2.23 (s, 3H), 3.06-3.24 (m, 6H), 3.25-3.36 (m, 2H), 3.46-3.65 (m+s, 9H), 4.19-4.29 (m, 2H), 6.75-6.82 (m, 1H), 6.96-7.06 (m, 1H), 7.63-7.72 (m, 1H), 8.78-8.96 (m, 1H), 8.79-8.92 (m, 1H), 9.88-10.04 (bs, 1H).

LC-MS (Method 2): Rₜ=0.93 min; MS (ESIpos): m/z=421.4 [M+H]⁺

### Intermediate 34

### tert-butyl 8-{3-cyano-7-[(2-methoxyethyl)(methyl)amino]-1-methyl-2-oxo-1,2-dihydro-quinolin-4-yl}-2,8-diazaspiro[4.5]decane-2-carboxylate

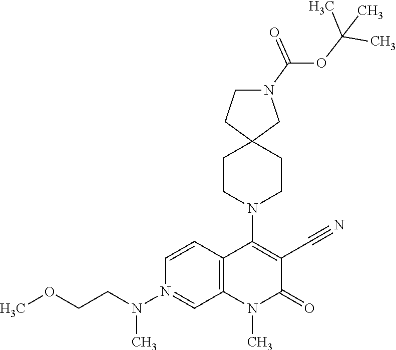

To a stirred solution of 500 mg tert-butyl 8-(7-bromo-3-cyano-1-methyl-2-oxo-1,2-dihydroquinolin-4-yl)-2,8-diazaspiro[4.5]decane-2-carboxylate (997 μmol, intermediate 31) in 10 mL 1,4-dioxane were added 260 μL 2-methoxy-N-methylethanamine (2.4 mmol, CAS 38256-93-8), 1.3 g cesium carbonate (3.99 mmol) and 157 mg chloro(2-dicyclohexylphosphino-2,4,6-triisopropyl-1,1-biphenyl)[2-(2-amino-1,1-biphenyl)]palladium(II) (199 μmol, CAS 1310584-14-5). The mixture was stirred for 2 h at 110° C. and 72 h at rt. The reaction mixture was diluted with water and ethyl acetate and the aqueous phase was extracted with ethyl acetate. The combined organic phases were washed with water and brine, filtered (using a water resistant filter) and concentrated under reduced pressure. The residue was purified by flash chromatography (silica, dichloromethane/methanol gradient 0-3%) to give 140 mg of the title compound (95% purity, 26% yield).

¹H NMR (400 MHz, DMSO-d₆) δ ppm 1.41 (s, 9H) 1.65-1.85 (m, 6H) 3.09 (s, 3H) 3.16-3.21 (m, 2H) 3.25 (s, 3H) 3.39-3.57 (m, 9H) 3.64-3.72 (m, 2H) 6.37-6.43 (m, 1H) 6.73-6.83 (m, 1H) 7.59-7.65 (m, 1H).

LC-MS (Method 2): Rₜ=1.31 min; MS (ESIpos): m/z=510.6 [M+H]⁺

### Intermediate 35

### 4-(2,8-diazaspiro[4.5]decan-8-yl)-7-[(2-methoxyethyl)(methyl)amino]-1-methyl-2-oxo-1,2-dihydroquinoline-3-carbonitrile, Salt with Trifluoracetic Acid

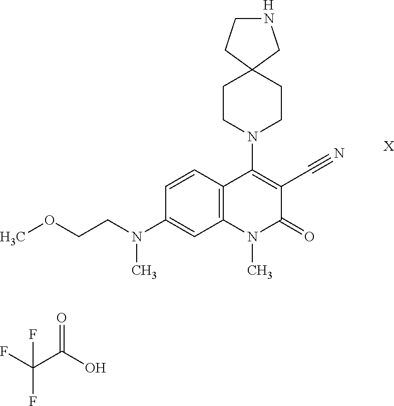

To a solution of 140 mg tert-butyl 8-{3-cyano-7-[(2-methoxyethyl)(methyl)amino]-1-methyl-2-oxo-1,2-dihydroquinolin-4-yl}-2,8-diazaspiro[4.5]decane-2-carboxylate (275 μmol, intermediate 34) in 3 mL dichloromethane was added 420 μL trifluoroacetic acid (5.5 mmol) and the mixture was stirred overnight at rt. The mixture was concentrated under reduced pressure and the residue was diluted with toluene (2×). The solvent was evaporated to give 110 mg of the title compound (90% purity, 88% yield).

¹H NMR (400 MHz, DMSO-d) δ ppm 1.71-1.86 (m, 4H), 1.87-1.95 (m, 2H), 3.06-3.15 (m, 5H), 3.22-3.27 (m, 3H), 3.27-3.35 (m, 2H), 3.44-3.58 (m, 9H), 3.65-3.73 (m, 2H), 6.37-6.43 (m, 1H), 6.75-6.82 (m, 1H), 7.59 (d, 1H), 8.74-8.86 (bs, 2H).

LC-MS (Method 2): Rₜ=1.03 min; MS (ESIpos): m/z=410.3 [M+H]⁺

### EXPERIMENTAL SECTION—EXAMPLES

### Example 1

### 1-methyl-2-oxo-4-(2-phenyl-2,8-diazaspiro[4.5]decan-8-yl)-1,2-dihydroquinoline-3-carbonitrile

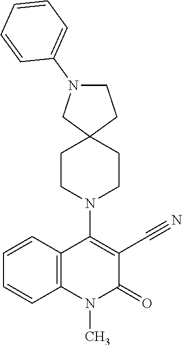 [SMILES: Cn1c(=O)c(C#N)c(N2CCC3(CCN(c4ccccc4)C3)CC2)c2ccccc21]

46 mg 2-phenyl-2,8-diazaspiro[4.5]decane hydrogen chloride salt (1:1) (182 μmol, intermediate 2), 39.8 mg 4-chloro-1-methyl-2-oxo-1,2-dihydroquinoline-3-carbonitrile (182 μmol, CAS 150617-68-8) and 160 μL N,N-diisopropylethylamine (910 μmol) was stirred in 3 mL dichloromethane for 45 min. at rt. Saturated aqueous sodium bicarbonate was added and the mixture was extracted with dichloromethane (3×). The organic phase was dried over magnesium sulfate, filtered and concentrated under reduced pressure. The residue was purified by flash chromatography (reverse phase, water (basic)/acetonitrile gradient 25-80%) to give 24.4 mg of the title compound (97% purity, 32% yield).

¹HNMR (400 MHz, CDCl₃) 1.86-1.96 (m, 4H), 2.03 (t, 2H), 3.30 (s, 2H), 3.43 (t, 2H), 3.58-3.73 (m, 4H), 3.68 (s, 3H), 6.58 (d, 2H), 6.70 (t, 1H), 7.22-7.28 (m, 3H), 7.37 (d, 1H), 7.62-7.67 (m, 1H), 7.83 (dd, 1H).

### Example 2

### 4-[2-(4-fluorophenyl)-2,8-diazaspiro[4.5]decan-8-yl]-1-methyl-2-oxo-1,2-dihydroquinoline-3-carbonitrile

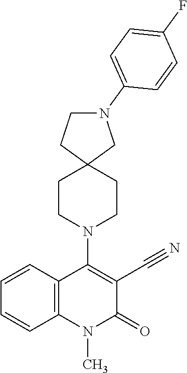 [SMILES: Cn1c(=O)c(C#N)c(N2CCC3(CCN(c4ccc(F)cc4)C3)CC2)c2ccccc21]

To a stirred solution of 100 mg 4-(2,8-diazaspiro[4.5]decan-8-yl)-1-methyl-2-oxo-1,2-dihydroquinoline-3-carbonitrile (295 μmol, intermediate 4) in 6 mL 1,4-dioxane were added 78 μL 1-bromo-4-fluorobenzene (710 μmol, CAS 460-00-4), 384 mg cesium carbonate (1.18 mmol) and 46.4 mg chloro(2-dicyclohexylphosphino-2,4,6-triisopropyl-1,1-biphenyl)[2-(2-amino-1,1-biphenyl)]palladium(II) (58.9 μmol, CAS 1310584-14-5). The mixture was stirred for 4 h at 110° C. The reaction mixture was diluted with water and ethyl acetate. The solid that precipitated from this procedure was collected by filtration. 85 mg of the title compound were obtained (90% purity, 62% yield).

¹H NMR (400 MHz, DMSO-d₆) δ ppm 1.75-1.89 (m, 4H) 1.93-2.03 (m, 2H) 3.19-3.25 (m, 2H) 3.29-3.33 (m, 2H) 3.51-3.71 (m, 7H) 6.50-6.60 (m, 2H) 6.95-7.08 (m, 2H) 7.31-7.36 (m, 1H) 7.53-7.60 (m, 1H) 7.68-7.78 (m, 1H) 7.84-7.92 (m, 1H).

LC-MS (Method 2): Rₜ=1.38 min; MS (ESIpos): m/z=417.4 [M+H]⁺

### Example 3

### 4-[2-(3,4-difluorophenyl)-2,8-diazaspiro[4.5]decan-8-yl]-1-methyl-2-oxo-1,2-dihydro-quinoline-3-carbonitrile

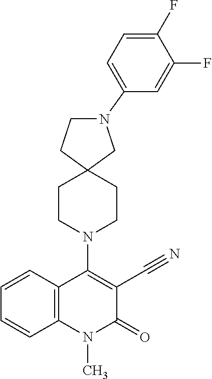 [SMILES: Cn1c(=O)c(C#N)c(N2CCC3(CCN(c4ccc(F)c(F)c4)C3)CC2)c2ccccc21]

113 mg 2-(3,4-difluorophenyl)-2,8-diazaspiro[4.5]decane hydrogen chloride salt (391 μmol, intermediate 6), 94.1 mg 4-chloro-1-methyl-2-oxo-1,2-dihydroquinoline-3-carbonitrile (430 μmol, CAS 150617-68-8) and 340 μL N,N-diisopropylethylamine (2 mmol) was stirred in 2 mL dichloromethane for 45 min. at rt. Saturated aqueous sodium bicarbonate was added and the mixture was extracted with dichloromethane (3×). The organic phase was dried over magnesium sulfate, filtered and concentrated under reduced pressure. The residue was purified by flash chromatography (reverse phase, water (basic)/acetonitrile gradient 25-75%) to give 71 mg of the title compound (97% purity, 41% yield).

¹HNMR (300 MHz, CDCl₃) 1.87-1.95 (m, 4H), 2.04 (t, 2H), 3.24 (s, 2H), 3.37 (t, 2H), 3.56-3.74 (m, 4H), 3.69 (s, 3H), 6.16-6.23 (m, 1H), 6.27-6.38 (m, 1H), 6.96-7.07 (m, 1H), 7.22-7.29 (m, 1H), 7.37 (d, 1H), 7.61-7.69 (m, 1H), 7.83 (dd, 1H).

### Example 4

### 4-[2-(4-fluorophenyl)-2,6-diazaspiro[3.4]octan-6-yl]-1-methyl-2-oxo-1,2-dihydroquinoline-3-carbonitrile

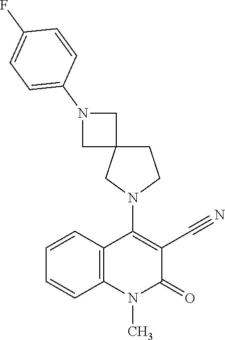 [SMILES: Cn1c(=O)c(C#N)c(N2CCC3(CN(c4ccc(F)cc4)C3)C2)c2ccccc21]

To a stirred solution of 200 mg 4-(2,6-diazaspiro[3.4]octan-6-yl)-1-methyl-2-oxo-1,2-dihydroquinoline-3-carbonitrile (645 μmol, intermediate 8) in 10 mL 1,4-dioxane were added 170 μL 1-bromo-4-fluorobenzene (1.5 mmol, CAS 460-00-4), 1.05 g cesium carbonate (3.23 mmol) and 102 mg chloro(2-dicyclohexylphosphino-2,4,6-triisopropyl-1,1-biphenyl)[2-(2-amino-1,1-biphenyl)]palladium(II) (129 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 140 mg of the title compound (97% purity, 54% yield).

¹H NMR (400 MHz, DMSO-d) δ ppm 2.23 (t, 2H) 3.48 (s, 3H) 3.72-3.83 (m, 4H) 4.02-4.09 (m, 2H) 4.15-4.26 (m, 2H) 6.37-6.48 (m, 2H) 6.96-7.08 (m, 2H) 7.19-7.28 (m, 1H) 7.42-7.51 (m, 1H) 7.62-7.70 (m, 1H) 8.03-8.12 (m, 1H).

LC-MS (Method 2): Rₜ=1.17 min; MS (ESIpos): m/z=389.5 [M+H]⁺

### Example 1

### 4-[2-(4-methoxyphenyl)-2,6-diazaspiro[3.4]octan-6-yl]-1-methyl-2-oxo-1,2-dihydro-quinoline-3-carbonitrile

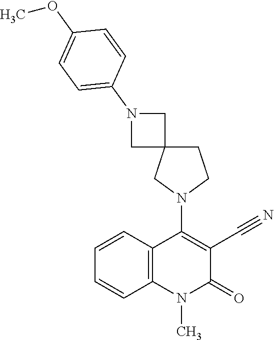 [SMILES: COc1ccc(N2CC3(CCN(c4c(C#N)c(=O)n(C)c5ccccc45)C3)C2)cc1]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 5 mL 1,4-dioxane were added 97 μL 1-bromo-4-methoxybenzene (770 μmol, CAS 104-92-7), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 2 h at 110° C. The reaction mixture was diluted with water and dichloromethane. The organic phase was washed with brine, filtered and was concentrated under reduced pressure. The residue was stirred in DMSO, the precipitate was collected by filtration, the solid was washed with methanol and dried in vacuo. 64 mg of the title compound were obtained (45% yield, 90% purity).

¹H NMR (400 MHz, DMSO-d₆) δ ppm 2.17-2.26 (m, 2H) 3.44-3.52 (m, 3H) 3.65 (s, 3H) 3.67-3.78 (m, 4H) 4.05 (t, 2H) 4.15-4.27 (m, 2H) 6.35-6.49 (m, 2H) 6.74-6.84 (m, 2H) 7.18-7.27 (m, 1H) 7.40-7.51 (m, 1H) 7.60-7.71 (m, 1H) 7.99-8.13 (m, 1H).

LC-MS (Method 2): Rₜ=1.12 min; MS (ESIpos): m/z=401.5 [M+H]⁺

### Example 6

### 4-[2-(4-chlorophenyl)-2,6-diazaspiro[3.4]octan-6-yl]-1-methyl-2-oxo-1,2-dihydroquinoline-3-carbonitrile

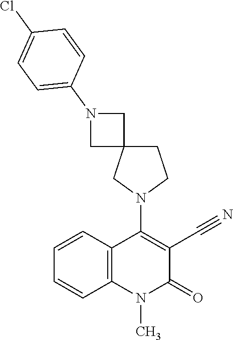 [SMILES: Cn1c(=O)c(C#N)c(N2CCC3(CN(c4ccc(Cl)cc4)C3)C2)c2ccccc21]

To a stirred solution of 200 mg 4-(2,6-diazaspiro[3.4]octan-6-yl)-1-methyl-2-oxo-1,2-dihydroquinoline-3-carbonitrile (645 μmol, intermediate 8) in 10 mL 1,4-dioxane were added 297 mg 1-bromo-4-chlorobenzene (1.55 mmol, CAS 106-39-8), 1.05 g cesium carbonate (3.23 mmol) and 102 mg chloro(2-dicyclohexylphosphino-2,4,6-triisopropyl-1,1-biphenyl)[2-(2-amino-1,1-biphenyl)]palladium(II) (129 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 130 mg of the title compound (96% purity, 48% yield).

¹H NMR (400 MHz, DMSO-d) δ ppm 2.23 (t, 2H) 3.47 (s, 3H) 3.75-3.86 (m, 4H) 3.98-4.12 (m, 2H) 4.15-4.28 (m, 2H) 6.38-6.47 (m, 2H) 7.15-7.31 (m, 3H) 7.43-7.50 (m, 1H) 7.61-7.71 (m, 1H) 8.02-8.09 (m, 1H).

LC-MS (Method 2): Rₜ=1.27 min; MS (ESIpos): m/z=405.4 [M+H]⁺

### Example 7

### 4-[6-(4-fluorophenyl)-2,6-diazaspiro[3.4]octan-2-yl]-1-methyl-2-oxo-1,2-dihydroquinoline-3-carbonitrile

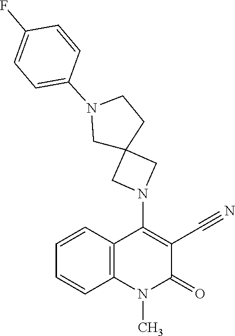 [SMILES: Cn1c(=O)c(C#N)c(N2CC3(CCN(c4ccc(F)cc4)C3)C2)c2ccccc21]

To a stirred solution of 100 mg 4-(2,6-diazaspiro[3.4]octan-2-yl)-1-methyl-2-oxo-1,2-dihydroquinoline-3-carbonitrile (323 μmol, intermediate 10) in 15 mL 1,4-dioxane were added 71 μL 1-bromo-4-fluorobenzene (650 μmol, CAS 460-00-4), 421 mg cesium carbonate (1.29 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 60 mg of the title compound (90% purity, 43% yield).

¹H NMR (400 MHz, DMSO-d₆) δ ppm 2.26-2.32 (m, 2H) 3.25-3.30 (m, 2H) 3.44-3.55 (m, 5H) 4.69-4.84 (m, 4H) 6.47-6.56 (m, 2H) 6.98-7.05 (m, 2H) 7.18-7.25 (m, 1H) 7.44-7.54 (m, 1H) 7.64-7.73 (m, 1H) 7.84-7.92 (m, 1H).

LC-MS (Method 2): Rₜ=1.21 min; MS (ESIpos): m/z=389.5 [M+H]⁺

### Example 8

### 1-methyl-2-oxo-4-{2-[4-(trifluoromethoxy)phenyl]-2,8-diazaspiro[4.5]decan-8-yl}-1,2-dihydroquinoline-3-carbonitrile

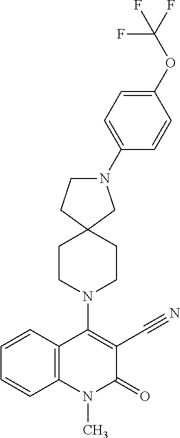 [SMILES: Cn1c(=O)c(C#N)c(N2CCC3(CCN(c4ccc(OC(F)(F)F)cc4)C3)CC2)c2ccccc21]

To a stirred solution of 100 mg 4-(2,8-diazaspiro[4.5]decan-8-yl)-1-methyl-2-oxo-1,2-dihydroquinoline-3-carbonitrile (295 μmol, intermediate 4) in 5 mL 1,4-dioxane were added 110 μL 1-bromo-4-(trifluoromethoxy)benzene (710 μmol, CAS 407-14-7), 384 mg cesium carbonate (1.18 mmol) and 46.4 mg chloro(2-dicyclohexylphosphino-2,4,6-triisopropyl-1,1-biphenyl)[2-(2-amino-1,1-biphenyl)]palladium(II) (58.9 μmol, CAS 1310584-14-5). The mixture was stirred for 4 h at 110° C. The reaction mixture was diluted with water and ethyl acetate. The organic phase was washed with brine, filtered and was concentrated under reduced pressure. The residue was purified by flash chromatography (silica, dichloromethane/methanol gradient 0-3%) to give 90 mg of the title compound (94% purity, 60% yield).

¹H NMR (400 MHz, DMSO-d₆) δ ppm 1.77-1.90 (m, 4H) 1.94-2.04 (m, 2H) 3.26 (s, 2H) 3.34-3.39 (m, 2H) 3.50-3.72 (m, 7H) 6.54-6.67 (m, 2H) 7.11-7.19 (m, 2H) 7.28-7.42 (m, 1H) 7.52-7.62 (m, 1H) 7.69-7.78 (m, 1H) 7.84-7.97 (m, 1H).

LC-MS (Method 2): Rₜ=1.49 min; MS (ESIpos): m/z=483.6 [M+H]⁺

### Example 9

### 4-[2-(3-methoxyphenyl)-2,8-diazaspiro[4.5]decan-8-yl]-1-methyl-2-oxo-1,2-dihydro-quinoline-3-carbonitrile

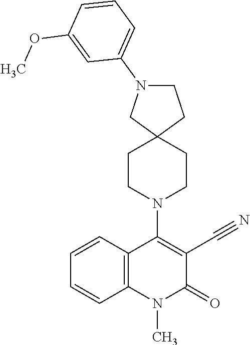 [SMILES: COc1cccc(N2CCC3(CCN(c4c(C#N)c(=O)n(C)c5ccccc45)CC3)C2)c1]

164 mg 2-(3-methoxyphenyl)-2,8-diazaspiro[4.5]decane hydrogen chloride salt (1:1) (580 μmol, intermediate 12), 127 mg 4-chloro-1-methyl-2-oxo-1,2-dihydroquinoline-3-carbonitrile (580 μmol, CAS 150617-68-8) and 400 μL triethylamine (2.9 mmol) was stirred in 4 mL dichloromethane for 1 h at rt. Saturated aqueous sodium bicarbonate was added and the mixture was extracted with dichloromethane (3×). The organic phase was dried over magnesium sulfate, filtered and concentrated under reduced pressure. The residue was purified by flash chromatography (reverse phase, water (basic)/acetonitrile gradient 25-80%) to give 140 mg of the title compound (98% purity, 55% yield).

¹HNMR (300 MHz, CDCl₃) 1.87-1.94 (m, 4H), 2.02 (t, 2H), 3.30 (s, 2H), 3.42 (t, 2H), 3.56-3.74 (m, 7H), 3.82 (s, 3H), 6.13 (t, 1H), 6.18-6.31 (m, 2H), 7.16 (t, 1H), 7.26 (m, 1H), 7.37 (d, 1H), 7.60-7.68 (m, 1H), 7.83 (dd, 1H).

### Example 10

### 4-[2-(2-methoxyphenyl)-2,8-diazaspiro[4.5]decan-8-yl]-1-methyl-2-oxo-1,2-dihydro-quinoline-3-carbonitrile

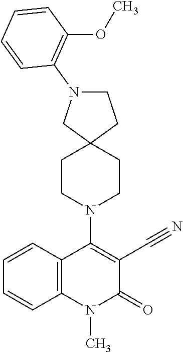 [SMILES: COc1ccccc1N1CCC2(CCN(c3c(C#N)c(=O)n(C)c4ccccc34)CC2)C1]

38 mg 2-(2-methoxyphenyl)-2,8-diazaspiro[4.5]decane hydrogen chloride salt (134 μmol, intermediate 14), 29.4 mg 4-chloro-1-methyl-2-oxo-1,2-dihydroquinoline-3-carbonitrile (134 μmol, CAS 150617-68-8) and 94 μL triethylamine (670 μmol) was stirred in 2.4 mL dichloromethane for 45 min. at rt. Saturated aqueous sodium bicarbonate was added and the mixture was extracted with dichloromethane (3×). The organic phase was dried over magnesium sulfate, filtered and concentrated under reduced pressure. The residue was purified by flash chromatography (reverse phase, water (basic)/acetonitrile gradient 30-80%) to give 43 mg of the title compound (98% purity, 73% yield).

¹HNMR (300 MHz, CDCl₃) 1.84-1.99 (m, 6H), 3.34 (s, 2H), 3.45 (t, 2H), 3.56-3.73 (m, 4H), 3.68 (s, 3H), 3.85 (s, 3H), 6.75 (d, 1H), 6.81-6.94 (m, 3H), 7.22-7.29 (m, 1H), 7.36 (d, 1H), 7.60-7.68 (m, 1H), 7.84 (d, 1H).

### Example 11

### 4-{2-[3-(dimethylamino)phenyl]-2,8-diazaspiro[4.5]decan-8-yl}-1-methyl-2-oxo-1,2-dihydroquinoline-3-carbonitrile

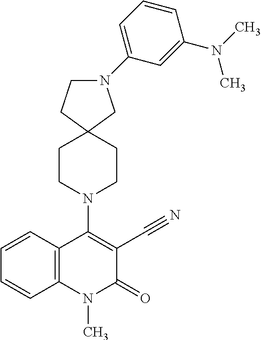 [SMILES: CN(C)c1cccc(N2CCC3(CCN(c4c(C#N)c(=O)n(C)c5ccccc45)CC3)C2)c1]

A solution of 98 mg 3-(2,8-diazaspiro[4.5]decan-2-yl)-N,N-dimethylaniline hydrogen chloride salt (295 μmol, intermediate 16), 64.5 mg 4-chloro-1-methyl-2-oxo-1,2-dihydroquinoline-3-carbonitrile (295 μmol, CAS 150617-68-8) and 260 μL N,N-diisopropylethylamine (1.5 mmol) was stirred in 5 mL DMSO for 18 h at 100° C. The mixture was cooled down to rt and was diluted with water. The mixture was extracted with ethyl acetate. The organic phase was washed with brine, dried over magnesium sulfate, filtered and concentrated under reduced pressure. The residue was purified by flash chromatography (reverse phase, water (basic)/acetonitrile gradient 15-90%). The impure product was further purified by mass directed auto purification to give 4 mg of the title compound (98% purity, 3% yield).

¹HNMR (CDCl₃, 400 MHz): 1.83-1.96 (m, 4H); 2.00 (t, 2H); 2.96 (s, 6H); 3.31 (s, 2H); 3.44 (t, 2H); 3.57-3.73 (m, 7H); 5.90 (m, 1H); 6.02 (dd, 1H); 6.17 (dd, 1H); 7.12 (t, 1H); 7.26 (t, 1H); 7.36 (d, 1H); 7.64 (t, 1H); 7.83 (d, 1H).

### Example 12

### 1-methyl-2-oxo-4-{2-[3-(trifluoromethoxy)phenyl]-2,8-diazaspiro[4.5]decan-8-yl}-1,2-dihydroquinoline-3-carbonitrile

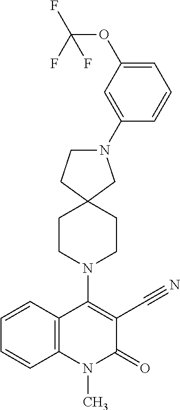 [SMILES: Cn1c(=O)c(C#N)c(N2CCC3(CCN(c4cccc(OC(F)(F)F)c4)C3)CC2)c2ccccc21]

To a stirred solution of 100 mg 4-(2,8-diazaspiro[4.5]decan-8-yl)-1-methyl-2-oxo-1,2-dihydroquinoline-3-carbonitrile (295 μmol, intermediate 4) in 5 mL 1,4-dioxane were added 170 mg 1-bromo-3-(trifluoromethoxy)benzene (707 μmol, CAS 2252-44-0), 384 mg cesium carbonate (1.18 mmol) and 46.4 mg chloro(2-dicyclohexylphosphino-2,4,6-triisopropyl-1,1-biphenyl)[2-(2-amino-1,1-biphenyl)]palladium(II) (58.9 μmol, CAS 1310584-14-5). The mixture was stirred for 5 h at 110° C. The reaction mixture was diluted with water and ethyl acetate. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 35 mg of the title compound (95% purity, 23% yield).

¹H NMR (400 MHz, DMSO-d₆) δ ppm 1.75-1.89 (m, 4H) 1.95-2.04 (m, 2H) 3.25-3.30 (m, 2H) 3.35-3.39 (m, 2H) 3.57 (s, 3H) 3.58-3.69 (m, 4H) 6.40-6.62 (m, 3H) 7.20-7.38 (m, 2H) 7.53-7.61 (m, 1H) 7.70-7.78 (m, 1H) 7.83-7.95 (m, 1H).

LC-MS (Method 2): Rₜ=1.52 min; MS (ESIpos): m/z=483.6 [M+H]⁺

### Example 13

### 4-{2-[2-(dimethylamino)phenyl]-2,8-diazaspiro[4.5]decan-8-yl}-1-methyl-2-oxo-1,2-dihydroquinoline-3-carbonitrile

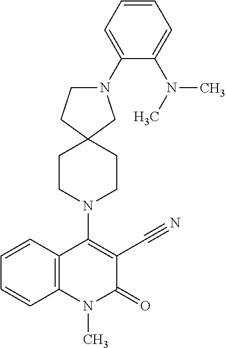 [SMILES: CN(C)c1ccccc1N1CCC2(CCN(c3c(C#N)c(=O)n(C)c4ccccc34)CC2)C1]

A solution of 129 mg 2-(2,8-diazaspiro[4.5]decan-2-yl)-N,N-dimethylaniline (497 μmol, intermediate 19), 109 mg 4-chloro-1-methyl-2-oxo-1,2-dihydroquinoline-3-carbonitrile (497 μmol, CAS 150617-68-8) and 430 μL N,N-diisopropylethylamine (2.5 mmol) was stirred in 10 mL DMSO for 18 h at 100° C. The mixture was cooled d own to rt and was diluted with water. The mixture was extracted with ethyl acetate. The organic phase was washed with brine, dried over magnesium sulfate, filtered and concentrated under reduced pressure. The residue was purified by flash chromatography (reverse phase, water (basic)/acetonitrile gradient 15-90%) to give 49 mg of the title compound (99% purity, 22% yield).

¹HNMR (CDCl₃, 400 MHz): 1.84-2.00 (m, 6H); 2.70 (s, 6H); 3.24 (s, 2H); 3.37 (t, 2H); 3.61-3.71 (m, 7H); 6.82 (d, 1H); 6.86 (t, 1H); 6.91-7.01 (m, 2H); 7.25 (m, 1H); 7.36 (d, 1H); 7.64 (t, 1H); 7.85 (d, 1H).

### Example 14

### 1-methyl-2-oxo-4-{2-[2-(trifluoromethoxy)phenyl]-2,8-diazaspiro[4.5]decan-8-yl}-1,2-dihydroquinoline-3-carbonitrile

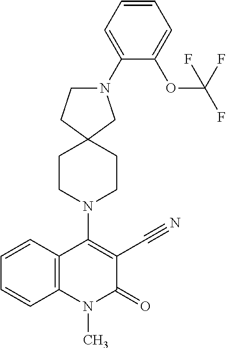 [SMILES: Cn1c(=O)c(C#N)c(N2CCC3(CCN(c4ccccc4OC(F)(F)F)C3)CC2)c2ccccc21]

To a stirred solution of 100 mg 4-(2,8-diazaspiro[4.5]decan-8-yl)-1-methyl-2-oxo-1,2-dihydroquinoline-3-carbonitrile (295 μmol, intermediate 4) in 5 mL 1,4-dioxane were added 170 mg 1-bromo-2-(trifluoromethoxy)benzene (707 μmol, CAS 64115-88-4), 384 mg cesium carbonate (1.18 mmol) and 46.4 mg chloro(2-dicyclohexylphosphino-2,4,6-triisopropyl-1,1-biphenyl)[2-(2-amino-1,1-biphenyl)]palladium(II) (58.9 μmol, CAS 1310584-14-5). The mixture was stirred for 5 h at 110° C. The reaction mixture was diluted with water and ethyl acetate. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 33 mg of the title compound (95% purity, 22% yield).

¹H NMR (400 MHz, DMSO-d) δ ppm 1.77-1.90 (m, 4H) 1.95 (t, 2H) 2.08 (s, 2H) 3.44-3.51 (m, 2H) 3.54-3.70 (m, 7H) 6.70-6.80 (m, 1H) 6.84-6.95 (m, 1H) 7.15-7.24 (m, 2H) 7.30-7.38 (m, 1H) 7.56 (dd, 1H) 7.73 (ddd, 1H) 7.89 (dd, 1H).

LC-MS (Method 2): Rₜ=1.52 min; MS (ESIpos): m/z=483.6 [M+H]⁺

### Example 15

### 4-[2-(4-methoxyphenyl)-2,8-diazaspiro[4.5]decan-8-yl]-1-methyl-2-oxo-1,2-dihydro-quinoline-3-carbonitrile

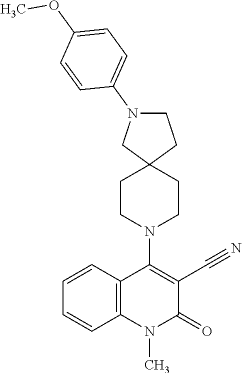 [SMILES: COc1ccc(N2CCC3(CCN(c4c(C#N)c(=O)n(C)c5ccccc45)CC3)C2)cc1]

A solution of 94 mg 2-(4-methoxyphenyl)-2,8-diazaspiro[4.5]decane hydrogen chloride salt (1:1) (332 μmol, intermediate 21), 72.7 mg 4-chloro-1-methyl-2-oxo-1,2-dihydroquinoline-3-carbonitrile (332 μmol, CAS 150617-68-8) and 290 μL N,N-diisopropylethylamine (1.7 mmol) was stirred in 3 mL DMSO for 2 h at rt. Saturated aqueous sodium bicarbonate was added and the mixture was extracted with dichloromethane (3×). The organic phase was dried over magnesium sulfate, filtered and concentrated under reduced pressure. The residue was purified by flash chromatography (reverse phase, water (basic)/acetonitrile gradient 25-75%) to give 71 mg of the title compound (95% purity, 47% yield).

1HNMR (400 MHz, CDCl3) 1.85-1.96 (m, 4H), 2.01 (t, 2H), 3.26 (s, 2H), 3.38 (t, 2H), 3.58-3.73 (m, 4H), 3.68 (s, 3H), 3.77 (s, 3H), 6.55 (d, 2H), 6.87 (d, 2H), 7.23-7.28 (m, 1H), 7.37 (d, 1H), 7.61-7.67 (m, 1H), 7.83 (dd, 1H).

### Example 16

### 4-[6-(4-fluorophenyl)-2,6-diazaspiro[3.5]nonan-2-yl]-1-methyl-2-oxo-1,2-dihydro-quinoline-3-carbonitrile

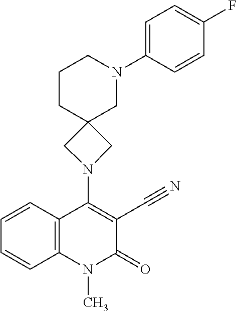 [SMILES: Cn1c(=O)c(C#N)c(N2CC3(CCCN(c4ccc(F)cc4)C3)C2)c2ccccc21]

To a stirred solution of 100 mg 4-(2,6-diazaspiro[3.5]nonan-2-yl)-1-methyl-2-oxo-1,2-dihydroquinoline-3-carbonitrile (308 μmol, intermediate 23) in 900 μL 1,4-dioxane were added 64.7 mg 1-bromo-4-fluorobenzene (370 μmol, CAS 460-00-4), 201 mg cesium carbonate (616 μmol) and 24.2 mg chloro(2-dicyclohexylphosphino-2,4,6-triisopropyl-1,1-biphenyl)[2-(2-amino-1,1-biphenyl)]palladium(II) (30.8 μmol, CAS 1310584-14-5). The mixture was stirred for 6 h at 110° C. The reaction mixture was diluted with water and extracted with ethyl acetate (3×). The organic phase was washed with water and brine, filtered and was concentrated under reduced pressure. The residue was purified by flash chromatography (silica, dichloromethane/methanol gradient 0-3%) to give 62 mg of the title compound (95% purity, 48% yield).

¹H NMR (400 MHz, DMSO-d₆) δ ppm 1.61-1.70 (m, 2H) 1.75-1.85 (m, 2H) 2.95 (br t, 2H) 3.18-3.27 (m, 2H) 3.49 (s, 3H) 4.50 (s, 4H) 7.04 (d, 4H) 7.16-7.29 (m, 1H) 7.47 (dd, 1H) 7.68 (ddd, 1H) 7.91 (dd, 1H).

LC-MS (Method 2): Rₜ=1.27 min; MS (ESIpos): m/z=403.6 [M+H]⁺

### Example 17

### 1-methyl-2-oxo-4-{2-[4-(trifluoromethoxy)phenyl]-2,6-diazaspiro[3.4]octan-6-yl}-1,2-dihydroquinoline-3-carbonitrile

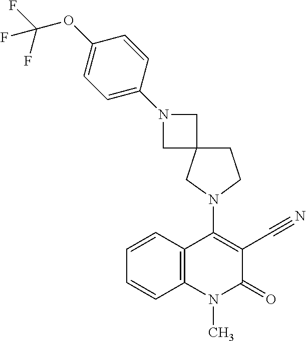 [SMILES: Cn1c(=O)c(C#N)c(N2CCC3(CN(c4ccc(OC(F)(F)F)cc4)C3)C2)c2ccccc21]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 4 mL 1,4-dioxane were added 120 μL 1-bromo-4-(trifluoromethoxy)benzene (770 μmol, CAS 407-14-7), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 3 h at 110° C. and 72 h at rt. The reaction mixture w as diluted with water and dichloromethane. The organic phase was washed with brine, filtered and was concentrated under reduced pressure. The residue was purified RP-HPLC (column: X-Bridge C18 5 μm 100×30 mm, mobile phase: acetonitrile/water (0.2 vol. % ammonia 32%)-gradient) to give 60 mg of the title compound (98% purity, 40% yield).

¹H NMR (400 MHz, DMSO-d₆) δ ppm 2.19-2.28 (m, 2H) 3.48 (s, 3H) 3.78-3.94 (m, 4H) 3.99-4.11 (m, 2H) 4.16-4.27 (m, 2H) 6.40-6.56 (m, 2H) 7.10-7.31 (m, 3H) 7.42-7.52 (m, 1H) 7.59-7.75 (m, 1H) 8.03-8.14 (m, 1H).

LC-MS (Method 2): Rₜ=1.33 min; MS (ESIpos): m/z=455.6 [M+H]⁺

### Example 18

### 1-methyl-2-oxo-4-{6-[4-(trifluoromethoxy)phenyl]-2,6-diazaspiro[3.4]octan-2-yl}-1,2-dihydroquinoline-3-carbonitrile

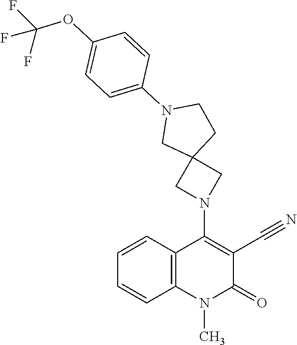 [SMILES: Cn1c(=O)c(C#N)c(N2CC3(CCN(c4ccc(OC(F)(F)F)cc4)C3)C2)c2ccccc21]

To a stirred solution of 100 mg 4-(2,6-diazaspiro[3.4]octan-2-yl)-1-methyl-2-oxo-1,2-dihydroquinoline-3-carbonitrile (323 μmol, intermediate 10) in 15 mL 1,4-dioxane were added 156 mg 1-bromo-4-(trifluoromethoxy)benzene (645 μmol, CAS 407-14-7), 315 mg cesium carbonate (968 μmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 50 mg of the title compound (90% purity, 31% yield).

¹H NMR (400 MHz, DMSO-d₆) δ ppm 2.26-2.33 (m, 2H) 3.32 (br s, 2H) 3.49 (s, 3H) 3.53-3.58 (m, 2H) 4.71-4.83 (m, 4H) 6.50-6.61 (m, 2H) 7.12-7.27 (m, 3H) 7.43-7.54 (m, 1H) 7.62-7.78 (m, 1H) 7.82-7.87 (m, 1H).

LC-MS (Method 2): Rₜ=1.36 min; MS (ESIpos): m/z=455.5 [M+H]⁺

### Example 19

### 1-methyl-2-oxo-4-{7-[4-(trifluoromethoxy)phenyl]-2,7-diazaspiro[4.4]nonan-2-yl}-1,2-dihydroquinoline-3-carbonitrile

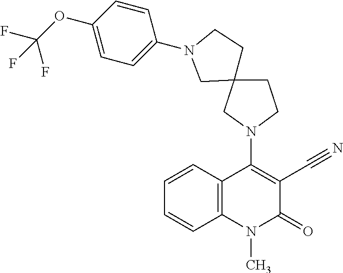 [SMILES: Cn1c(=O)c(C#N)c(N2CCC3(CCN(c4ccc(OC(F)(F)F)cc4)C3)C2)c2ccccc21]

To a stirred solution of 100 mg 4-(2,7-diazaspiro[4.4]nonan-2-yl)-1-methyl-2-oxo-1,2-dihydroquinoline-3-carbonitrile (308 μmol, intermediate 25) in 4 mL 1,4-dioxane were added 110 μL 1-bromo-4-(trifluoromethoxy)benzene (740 μmol, CAS 407-14-7), 401 mg cesium carbonate (1.23 mmol) and 48.5 mg chloro(2-dicyclohexylphosphino-2,4,6-triisopropyl-1,1-biphenyl)[2-(2-amino-1,1-biphenyl)]palladium(II) (61.6 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified RP-HPLC (column: X-Bridge C18 5 μm 100×30 mm, mobile phase: acetonitrile/water (0.2 vol. % ammonia 32%)-gradient) to give 70 mg of the title compound (88% purity, 43% yield).

¹H NMR (400 MHz, DMSO-d₆) δ ppm 2.28-2.33 (m, 2H) 3.32 (br s, 2H) 3.44-3.58 (m, 5H) 4.70-4.86 (m, 4H) 6.50-6.62 (m, 2H) 7.11-7.27 (m, 3H) 7.43-7.51 (m, 1H) 7.63-7.76 (m, 1H) 7.81-7.94 (m, 1H).

LC-MS (Method 2): Rₜ=1.36 min; MS (ESIpos): m/z=469.3 [M+H]⁺

### Example 20

### 4-[2-(3-methoxyphenyl)-2,6-diazaspiro[3.4]octan-6-yl]-1-methyl-2-oxo-1,2-dihydroquinoline-3-carbonitrile

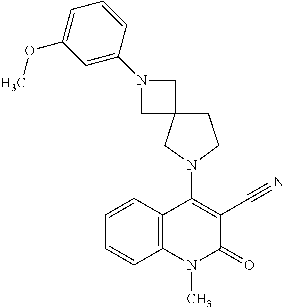 [SMILES: COc1cccc(N2CC3(CCN(c4c(C#N)c(=O)n(C)c5ccccc45)C3)C2)c1]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 5 mL 1,4-dioxane were added 98 μL 1-bromo-3-methoxybenzene (770 μmol, CAS 2398-37-0), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 2 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 80 mg of the title compound (95% purity, 59% yield).

¹H NMR (400 MHz, DMSO-d) δ ppm 2.23 (t, 2H) 3.48 (s, 3H) 3.68 (s, 3H) 3.75-3.83 (m, 4H) 4.06 (t, 2H) 4.20 (s, 2H) 5.96 (t, 1H) 6.00-6.04 (m, 1H) 6.23-6.32 (m, 1H) 7.01-7.11 (m, 1H) 7.19-7.27 (m, 1H) 7.43-7.51 (m, 1H) 7.62-7.70 (m, 1H) 8.04-8.10 (m, 1H).

LC-MS (Method 2): Rₜ=1.16 min; MS (ESIpos): m/z=401.5 [M+H]⁺

### Example 21

### 4-{2-[4-(dimethylamino)phenyl]-2,6-diazaspiro[3.4]octan-6-yl}-1-methyl-2-oxo-1,2-dihydroquinoline-3-carbonitrile

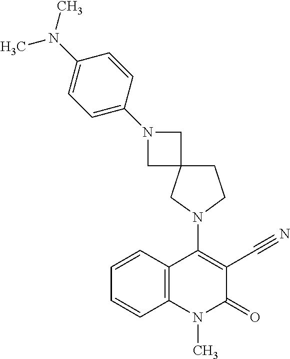 [SMILES: CN(C)c1ccc(N2CC3(CCN(c4c(C#N)c(=O)n(C)c5ccccc45)C3)C2)cc1]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 5 mL 1,4-dioxane were added 129 mg 4-bromo-N,N-dimethylaniline (645 μmol, CAS 586-77-6), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 2 h at 110° C. The reaction mixture was diluted with water and dichloromethane. The organic phase was washed with brine, filtered and was concentrated under reduced pressure. The residue was stirred in DMSO, the precipitate was collected by filtration and dried in vacuo. 50 mg of the title compound were obtained (34% yield, 90% purity).

¹H NMR (400 MHz, DMSO-d) δ ppm 2.21 (t, 2H) 2.70-2.77 (m, 6H) 3.48 (s, 3H) 3.63-3.75 (m, 4H) 4.00-4.10 (m, 2H) 4.19 (s, 2H) 6.34-6.42 (m, 2H) 6.63-6.71 (m, 2H) 7.19-7.27 (m, 1H) 7.41-7.50 (m, 1H) 7.62-7.69 (m, 1H) 8.02-8.13 (m, 1H).

LC-MS (Method 2): Rₜ=1.14 min; MS (ESIpos): m/z=414.6 [M+H]⁺

### Example 22

### 1-methyl-2-oxo-4-{2-[4-(2-oxopyrrolidin-1-yl)phenyl]-2,6-diazaspiro[3.4]octan-6-yl}-1,2-dihydroquinoline-3-carbonitrile

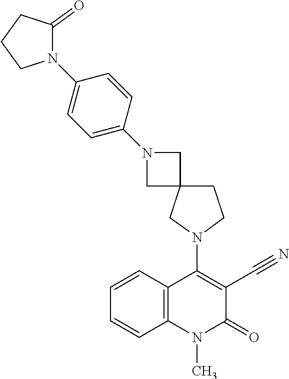 [SMILES: Cn1c(=O)c(C#N)c(N2CCC3(CN(c4ccc(N5CCCC5=O)cc4)C3)C2)c2ccccc21]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 5 mL 1,4-dioxane were added 155 mg 1-(4-bromophenyl)pyrrolidin-2-one (645 μmol, CAS 7661-32-7), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 3 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%) to give 50 mg of the title compound (95% purity, 32% yield).

¹H NMR (400 MHz, DMSO-d₆) δ ppm 1.97-2.07 (m, 2H) 2.17-2.26 (m, 2H) 2.38-2.45 (m, 2H) 3.48 (s, 3H) 3.66-3.88 (m, 6H) 4.06 (t, 2H) 4.15-4.25 (m, 2H) 6.39-6.51 (m, 2H) 7.18-7.29 (m, 1H) 7.37-7.50 (m, 3H) 7.61-7.71 (m, 1H) 8.01-8.14 (m, 1H).

LC-MS (Method 2): Rₜ=1.00 min; MS (ESIpos): m/z=454.6 [M+H]⁺

### Example 23

### 4-[7-(4-fluorophenyl)-2,7-diazaspiro[4.4]nonan-2-yl]-1-methyl-2-oxo-1,2-dihydro-quinoline-3-carbonitrile

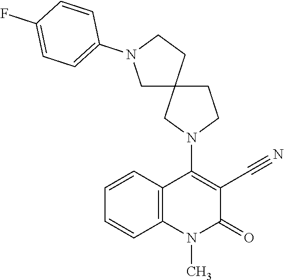 [SMILES: Cn1c(=O)c(C#N)c(N2CCC3(CCN(c4ccc(F)cc4)C3)C2)c2ccccc21]

To a stirred solution of 100 mg 4-(2,7-diazaspiro[4.4]nonan-2-yl)-1-methyl-2-oxo-1,2-dihydroquinoline-3-carbonitrile (308 μmol, intermediate 25) in 4 mL 1,4-dioxane were added 129 mg 1-bromo-4-fluorobenzene (739 μmol, CAS 460-00-4), 201 mg cesium carbonate (616 μmol) and 48.5 mg chloro(2-dicyclohexylphosphino-2,4,6-triisopropyl-1,1-biphenyl)[2-(2-amino-1,1-biphenyl)]palladium(II) (61.6 μmol, CAS 1310584-14-5). The mixture was stirred for 4 h at 110° C. The reaction mixture was diluted with water and dichloromethane. The organic phase was washed with brine, filtered and was concentrated under reduced pressure. The residue was purified RP-HPLC (column: X-Bridge C18 5 μm 100×30 mm, mobile phase: acetonitrile/water (0.2 vol. % ammonia 32%)-gradient) to give 68 mg of the title compound (85% purity, 47% yield).

¹H NMR (400 MHz, DMSO-d₆) δ ppm 1.95-2.13 (m, 4H) 3.19-3.32 (m, 4H) 3.47 (s, 3H) 3.93-4.04 (m, 2H) 4.09-4.20 (m, 2H) 6.45-6.56 (m, 2H) 6.94-7.06 (m, 2H) 7.18-7.30 (m, 1H) 7.40-7.49 (m, 1H) 7.58-7.72 (m, 1H) 8.04-8.16 (m, 1H).

LC-MS (Method 2): Rₜ=1.32 min; MS (ESIpos): m/z=403.3 [M+H]⁺

### Example 24

### 1-methyl-4-{2-[4-(morpholin-4-yl)phenyl]-2,6-diazaspiro[3.4]octan-6-yl}-2-oxo-1,2-dihydroquinoline-3-carbonitrile

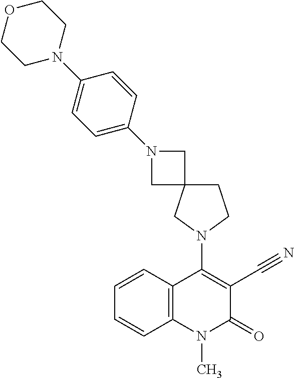 [SMILES: Cn1c(=O)c(C#N)c(N2CCC3(CN(c4ccc(N5CCOCC5)cc4)C3)C2)c2ccccc21]

To a stirred solution of 100 mg 4-(2,6-diazaspiro[3.4]octan-6-yl)-1-methyl-2-oxo-1,2-dihydroquinoline-3-carbonitrile (323 μmol, intermediate 8) in 5 mL 1,4-dioxane were added 156 mg 4-(4-bromophenyl)morpholine (645 μmol, CAS 30483-75-1), 526 mg cesium carbonate (1.61 mmol) and 50.8 mg chloro(2-dicyclohexylphosphino-2,4,6-triisopropyl-1,1-biphenyl)[2-(2-amino-1,1-biphenyl)]palladium(II) (64.5 μmol, CAS 1310584-14-5). The mixture was stirred for 3 h at 110° C. The reaction mixture was diluted with water and dichloromethane. The organic phase was washed with brine, filtered and was concentrated under reduced pressure. The residue was purified by flash chromatography (silica, dichloromethane/methanol gradient 0-3%). The impure product was refluxed in methanol for some time. The solid that precipitated from this procedure was collected by filtration to give 67 mg of the title compound (95% purity, 46% yield).

¹H NMR (400 MHz, DMSO-d₆) δ ppm 2.16-2.27 (m, 2H) 2.87-2.97 (m, 4H) 3.48 (s, 3H) 3.67-3.78 (m, 8H) 3.99-4.10 (m, 2H) 4.16-4.24 (m, 2H) 6.35-6.42 (m, 2H) 6.80-6.89 (m, 2H) 7.23 (td, 1H) 7.46 (dd, 1H) 7.66 (ddd, 1H) 8.07 (dd, 1H).

LC-MS (Method 2): Rₜ=1.05 min; MS (ESIpos): m/z=456.6 [M+H]⁺

### Example 25

### 1-methyl-2-oxo-4-{2-[4-(trifluoromethoxy)phenyl]-2,8-diazaspiro[4.5]decan-8-yl}-1,2-dihydroquinoline-3-carboxamide

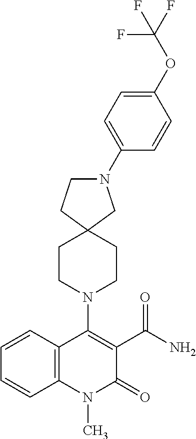 [SMILES: Cn1c(=O)c(C(N)=O)c(N2CCC3(CCN(c4ccc(OC(F)(F)F)cc4)C3)CC2)c2ccccc21]

183 mg 1-methyl-2-oxo-4-{2-[4-(trifluoromethoxy)phenyl]-2,8-diazaspiro[4.5]decan-8-yl}-1,2-dihydroquinoline-3-carbonitrile (361 μmol, example 8), 20.3 mg palladium(II)acetate (90.2 μmol) and 214 mg acetaldoxime (3.6 mmol) were stirred in 5.0 mL ethanol for 6 h at 80° C. The reaction mixture was diluted with water, extracted with ethyl acetate (2×), the combined organic layers were washed with brine, filtered through a water resistant filter and the filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 24 mg of the title compound (95% purity, 13% yield).

¹H NMR (400 MHz, DMSO-d) δ ppm 1.77 (br s, 4H) 1.94 (t, 2H) 3.07-3.27 (m, 6H) 3.33-3.37 (m, 2H) 3.58 (s, 3H) 6.48-6.63 (m, 2H) 7.11-7.19 (m, 2H) 7.30 (td, 1H) 7.44-7.56 (m, 2H) 7.57-7.72 (m, 2H) 7.93 (dd, 1H).

LC-MS (Method 2): Rₜ=1.40 min; MS (ESIpos): m/z=501.6 [M+H]⁺

### Example 26

### 4-[2-(4-chlorophenyl)-2,6-diazaspiro[3.4]octan-6-yl]-1-methyl-2-oxo-1,2-dihydroquinoline-3-carboxamide

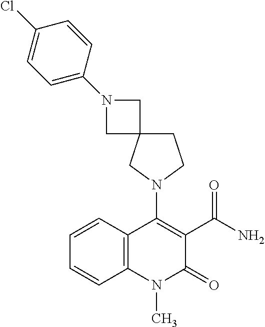 [SMILES: Cn1c(=O)c(C(N)=O)c(N2CCC3(CN(c4ccc(Cl)cc4)C3)C2)c2ccccc21]

80 mg 4-[2-(4-chlorophenyl)-2,6-diazaspiro[3.4]octan-6-yl]-1-methyl-2-oxo-1,2-dihydro-quinoline-3-carbonitrile (198 μmol, example 6), 22.2 mg palladium(II)acetate (98.8 μmol) and 117 mg acetaldoxime (1.9 mmol) were stirred in 3.0 mL ethanol for 5 h at 80° C. The reaction mixture was concentrated under reduced pressure. The residue was purified by flash chromatography (silica, dichloromethane/methanol gradient 0-5%). The impure product was purified by RP-HPLC (column: X-Bridge C18 5 μm 100×30 mm, mobile phase: acetonitrile/water (0.2 vol. % ammonia 32%)-gradient) to give 22 mg of the title compound (95% purity, 25% yield).

¹H NMR (400 MHz, DMSO-d) δ ppm 2.18 (t, 2H) 3.53 (s, 3H) 3.60 (t, 2H) 3.74-3.82 (m, 6H) 6.40-6.51 (m, 2H) 7.13-7.30 (m, 4H) 7.43-7.52 (m, 1H) 7.53-7.64 (m, 1H) 7.89-7.99 (m, 1H) 8.00-8.12 (m, 1H).

LC-MS (Method 2): Rₜ=1.17 min; MS (ESIpos): m/z=423.5 [M+H]⁺

### Example 27

### 4-[2-(4-fluorophenyl)-2,6-diazaspiro[3.4]octan-6-yl]-1-methyl-2-oxo-1,2-dihydroquinoline-3-carboxamide

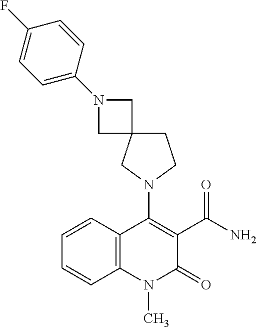 [SMILES: Cn1c(=O)c(C(N)=O)c(N2CCC3(CN(c4ccc(F)cc4)C3)C2)c2ccccc21]

80 mg 4-[2-(4-fluorophenyl)-2,6-diazaspiro[3.4]octan-6-yl]-1-methyl-2-oxo-1,2-dihydroquinoline-3-carbonitrile (206 μmol, example 4), 23.2 mg palladium(II)acetate (0.1 mmol) and 120.6 mg acetaldoxime (2.06 mmol) were stirred in 5.0 mL ethanol for 5 h at 80° C. The reaction mixture was concentrated under reduced pressure. The residue was purified by flash chromatography (silica, dichloromethane/methanol gradient 0-5%). The impure product was purified by RP-HPLC (column: X-Bridge C18 5 μm 100×30 mm, mobile phase: acetonitrile/water (0.2 vol. % ammonia 32%)-gradient) to give 6 mg of the title compound (95% purity, 7% yield).

¹H NMR (400 MHz, DMSO-d) δ ppm 2.18 (t, 2H) 3.53 (s, 3H) 3.60 (t, 2H) 3.73-3.81 (m, 6H) 6.40-6.48 (m, 2H) 6.96-7.05 (m, 2H) 7.18-7.26 (m, 2H) 7.44-7.50 (m, 1H) 7.55-7.62 (m, 1H) 7.91-8.01 (m, 1H) 8.05-8.14 (m, 1H).

LC-MS (Method 2): Rₜ=1.08 min; MS (ESIpos): m/z=407.5 [M+H]⁺

### Example 28

### 4-{2-[4-(dimethylamino)phenyl]-2,6-diazaspiro[3.4]octan-6-yl}-1-methyl-2-oxo-1,2-dihydroquinoline-3-carboxamide

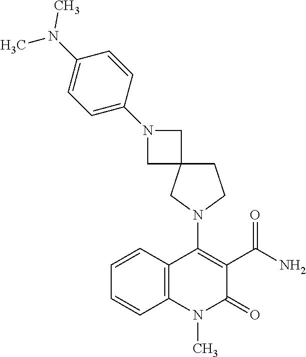 [SMILES: CN(C)c1ccc(N2CC3(CCN(c4c(C(N)=O)c(=O)n(C)c5ccccc45)C3)C2)cc1]

50 mg 4-{2-[4-(dimethylamino)phenyl]-2,6-diazaspiro[3.4]octan-6-yl}-1-methyl-2-oxo-1,2-dihydroquinoline-3-carbonitrile (121 μmol, example 21), 6.79 mg palladium(II)acetate (30.2 μmol) and 35.7 mg acetaldoxime (605 μmol) were stirred in 2.0 mL ethanol for 3 h at 80° C. The reaction mixture was concentrated under reduced pressure. Water was added and the mixture was extracted with ethyl acetate (3×). The organic phase was washed with water and brine, filtered and concentrated under reduced pressure. The residue was purified by flash chromatography (silica, dichloromethane/methanol gradient 0-10%) to give 9 mg of the title compound (95% purity, 16% yield).

¹H NMR (400 MHz, CHLOROFORM-d) δ ppm 2.22 (t, 2H) 2.84 (s, 6H) 3.61 (s, 3H) 3.69-3.82 (m, 4H) 3.87-3.95 (m, 2H) 4.00-4.08 (m, 2H) 5.33-5.48 (m, 1H) 6.37-6.54 (m, 2H) 6.70-6.83 (m, 2H) 7.11-7.23 (m, 1H) 7.29-7.34 (m, 1H) 7.46-7.59 (m, 1H) 7.85 (dd, 1H) 9.20 (br d, 1H).

LC-MS (Method 2): Rₜ=1.04 min; MS (ESIpos): m/z=432.5 [M+H]⁺

### Example 29

### 7-bromo-1-methyl-2-oxo-4-{2-[4-(trifluoromethoxy)phenyl]-2,6-diazaspiro[3.4]octan-6-yl}-1,2-dihydroquinoline-3-carbonitrile

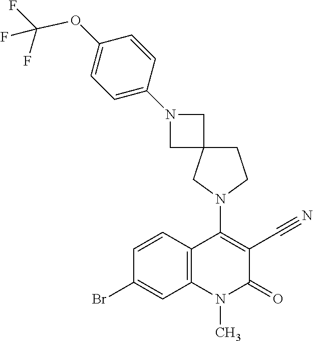 [SMILES: Cn1c(=O)c(C#N)c(N2CCC3(CN(c4ccc(OC(F)(F)F)cc4)C3)C2)c2ccc(Br)cc21]

To a stirred solution of 200 mg 7-bromo-4-(2,6-diazaspiro[3.4]octan-6-yl)-1-methyl-2-oxo-1,2-dihydroquinoline-3-carbonitrile (509 μmol, intermediate 30) in 8 mL 1,4-dioxane were added 180 μL 1-bromo-4-(trifluoromethoxy)benzene (1.2 mmol, CAS 407-14-7), 829 mg cesium carbonate (2.55 mmol) and 80.1 mg chloro(2-dicyclohexylphosphino-2,4,6-triisopropyl-1,1-biphenyl)[2-(2-amino-1,1-biphenyl)]palladium(II) (102 μmol, CAS 1310584-14-5). The mixture was stirred for 3 h at 110° C. and 72 h at rt. The reaction mixture was diluted with water and dichloromethane. The organic phase was washed with brine, filtered and was concentrated under reduced pressure.

The residue was purified by RP-HPLC (column: X-Bridge C18 5 μm 100×30 mm, mobile phase: acetonitrile/water (0.2 vol. % ammonia 32%)-gradient) to give 40 mg of the title compound (97% purity, 14% yield).

¹H NMR (400 MHz, DMSO-d₆) δ ppm 2.17-2.30 (m, 2H) 3.47 (s, 3H) 3.79-3.89 (m, 4H) 4.01-4.08 (m, 2H) 4.17-4.25 (m, 2H) 6.43-6.51 (m, 2H) 7.16 (d, 2H) 7.38 (dd, 1H) 7.61-7.71 (m, 1H) 7.93-7.99 (m, 1H).

LC-MS (Method 2): Rₜ=1.39 min; MS (ESIpos): m/z=535.4 [M+H]⁺

### Example 30

### 7-bromo-4-[2-(4-chlorophenyl)-2,6-diazaspiro[3.4]octan-6-yl]-1-methyl-2-oxo-1,2-dihydroquinoline-3-carbonitrile

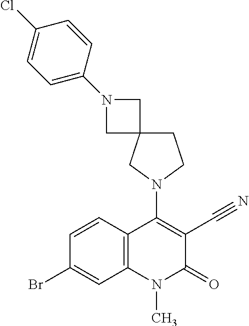 [SMILES: Cn1c(=O)c(C#N)c(N2CCC3(CN(c4ccc(Cl)cc4)C3)C2)c2ccc(Br)cc21]

To a stirred solution of 200 mg 7-bromo-4-(2,6-diazaspiro[3.4]octan-6-yl)-1-methyl-2-oxo-1,2-dihydroquinoline-3-carbonitrle (509 μmol, intermediate 30) in 10 mL 1,4-dioxane were added 234 mg 1-bromo-4-chlorobenzene (1.22 mmol, CAS 106-39-8), 829 mg cesium carbonate (2.55 mmol) and 80.1 mg chloro(2-dicyclohexylphosphino-2,4,6-triisopropyl-1,1-biphenyl)[2-(2-amino-1,1-biphenyl)]palladium(II) (102 μmol, CAS 1310584-14-5). The mixture was stirred for 1 h at 110° C. and 18 h at rt. The reaction mixture was diluted with water and dichloromethane. The organic phase was washed with brine, filtered and was concentrated under reduced pressure. The residue was purified by flash chromatography (silica, dichloromethane/methanol gradient 0-2%) to give 40 mg of the title compound (94% purity, 15% yield).

¹H NMR (400 MHz, DMSO-d₆) δ ppm 2.17-2.27 (m, 2H) 3.47 (s, 3H) 3.75-3.84 (m, 4H) 4.04 (t, 2H) 4.19 (s, 2H) 6.40-6.48 (m, 2H) 7.13-7.23 (m, 2H) 7.34-7.41 (m, 1H) 7.61-7.68 (m, 1H) 7.93-8.02 (m, 1H).

LC-MS (Method 2): Rₜ=1.37 min; MS (ESIpos): m/z=483.4 [M+H]⁺

### Example 31

### 1-methyl-7-(4-methylpiperazin-1-yl)-2-oxo-4-{2-[3-(trifluoromethoxy)phenyl]-2,8-diazaspiro[4.5]decan-8-yl}-1,2-dihydroquinoline-3-carbonitrile

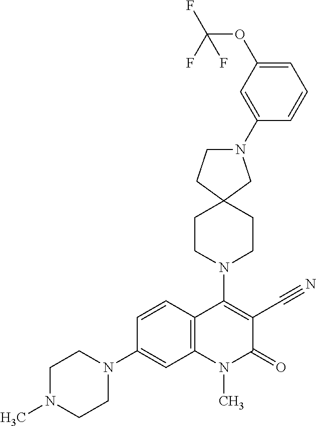 [SMILES: CN1CCN(c2ccc3c(N4CCC5(CCN(c6cccc(OC(F)(F)F)c6)C5)CC4)c(C#N)c(=O)n(C)c3c2)CC1]

To a stirred solution of 50 mg 4-(2,8-diazaspiro[4.5]decan-8-yl)-1-methyl-7-(4-methylpiperazin-1-yl)-2-oxo-1,2-dihydroquinoline-3-carbonitrile (119 μmol, intermediate 33) in 3 mL 1,4-dioxane were added 42 μL 1-bromo-3-(trifluoromethoxy)benzene (290 μmol, CAS 2252-44-0), 155 mg cesium carbonate (476 μmol) and 18.7 mg chloro(2-dicyclohexylphosphino-2,4,6-triisopropyl-1,1-biphenyl)[2-(2-amino-1,1-biphenyl)]palladium(II) (23.8 μmol, CAS 1310584-14-5). The mixture was stirred for 5 h at 110° C. and 72 h at rt. The reaction mixture was diluted with water and dichloromethane. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 45 mg of the title compound (98% purity, 64% yield).

¹H NMR (400 MHz, DMSO-d₆) δ ppm 1.80 (br t, 4H) 1.98 (t, 2H) 2.23 (s, 3H) 2.41-2.47 (m, 4H) 3.22-3.28 (m, 2H) 3.35-3.40 (m, 2H) 3.40-3.47 (m, 4H) 3.49-3.64 (m, 7H) 6.42-6.47 (m, 1H) 6.48-6.61 (m, 2H) 6.63-6.70 (m, 1H) 6.92-7.01 (m, 1H) 7.20-7.32 (m, 1H) 7.59-7.69 (m, 1H).

LC-MS (Method 2): Rₜ=1.48 min; MS (ESIpos): m/z=581.7 [M+H]⁺

### Example 32

### 7-[(2-methoxyethyl)(methyl)amino]-1-methyl-2-oxo-4-{2-[3-(trifluoromethoxy)phenyl]-2,8-diazaspiro[4.5]decan-8-yl}-1,2-dihydroquinoline-3-carbonitrile

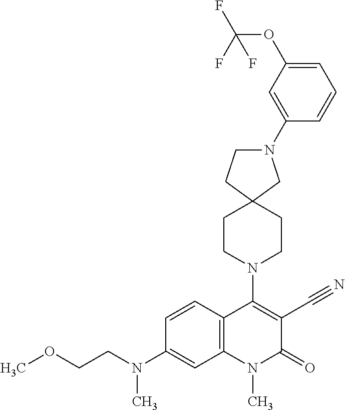 [SMILES: COCCN(C)c1ccc2c(N3CCC4(CCN(c5cccc(OC(F)(F)F)c5)C4)CC3)c(C#N)c(=O)n(C)c2c1]

To a stirred solution of 55 mg 4-(2,8-diazaspiro[4.5]decan-8-yl)-7-[(2-methoxyethyl)-(methyl)amino]-1-methyl-2-oxo-1,2-dihydroquinoline-3-carbonitrle (134 μmol, intermediate 35) in 3 mL 1,4-dioxane were added 48 μL 1-bromo-3-(trifluoromethoxy)benzene (320 μmol, CAS 2252-44-0), 175 mg cesium carbonate (537 μmol) and 21.1 mg chloro(2-dicyclohexylphosphino-2,4,6-triisopropyl-1,1-biphenyl)[2-(2-amino-1,1-biphenyl)]palladium(II) (26.9 μmol, CAS 1310584-14-5). The mixture was stirred for 5 h at 110° C. and 72 h at rt. The reaction mixture was diluted with water and dichloromethane. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 40 mg of the title compound (98% purity, 51% yield).

¹H NMR (400 MHz, DMSO-d₆) δ ppm 1.80 (br t, 4H) 1.98 (t, 2H) 3.09 (s, 3H) 3.26 (s, 5H) 3.34-3.39 (m, 2H) 3.47-3.61 (m, 9H) 3.63-3.74 (m, 2H) 6.38-6.46 (m, 2H) 6.47-6.63 (m, 2H) 6.74-6.85 (m, 1H) 7.25 (t, 1H) 7.63 (d, 1H).

LC-MS (Method 2): Rₜ=1.53 min; MS (ESIpos): m/z=570.7 [M+H]⁺

### Example 33

### 4-[2-(3-chlorophenyl)-2,8-diazaspiro[4.5]decan-8-yl]-1-methyl-7-(4-methylpiperazin-1-yl)-2-oxo-1,2-dihydroquinoline-3-carbonitrile

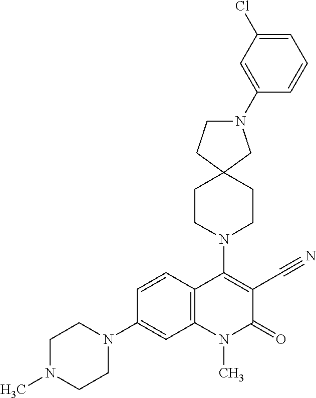 [SMILES: CN1CCN(c2ccc3c(N4CCC5(CCN(c6cccc(Cl)c6)C5)CC4)c(C#N)c(=O)n(C)c3c2)CC1]

To a stirred solution 50 mg 4-(2,8-diazaspiro[4.5]decan-8-yl)-1-methyl-7-(4-methylpiperazin-1-yl)-2-oxo-1,2-dihydroquinoline-3-carbonitrile (119 μmol, intermediate 33) in 3 mL 1,4-dioxane were added 34 μL 1-bromo-3-chlorobenzene (290 μmol, CAS 108-37-2), 155 mg cesium carbonate (476 μmol) and 18.7 mg chloro(2-dicyclohexylphosphino-2,4,6-triisopropyl-1,1-biphenyl)[2-(2-amino-1,1-biphenyl)]palladium(II) (23.8 μmol, CAS 1310584-14-5). The mixture was stirred for 5 h at 110° C. The reaction mixture was diluted with water and ethyl acetate. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 40 mg of the title compound (98% purity, 62% yield).

¹H NMR (400 MHz, DMSO-d₆) δ ppm 1.79 (br t, 4H) 1.97 (t, 2H) 2.23 (s, 3H) 2.45 (br s, 4H) 3.25 (s, 2H) 3.34-3.37 (m, 2H) 3.40-3.47 (m, 4H) 3.49-3.63 (m, 7H) 6.47-6.61 (m, 3H) 6.63-6.71 (m, 1H) 6.94-7.02 (m, 1H) 7.10-7.19 (m, 1H) 7.61-7.68 (m, 1H).

LC-MS (Method 2): Rₜ=1.40 min; MS (ESIpos): m/z=531.7 [M+H]⁺

### Example 34

### 4-[2-(3-chlorophenyl)-2,8-diazaspiro[4.5]decan-8-yl]-7-[(2-methoxyethyl)(methyl)amino]-1-methyl-2-oxo-1,2-dihydroquinoline-3-carbonitrile

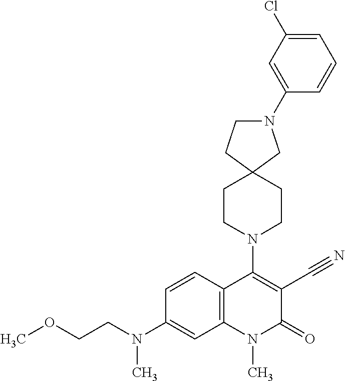 [SMILES: COCCN(C)c1ccc2c(N3CCC4(CCN(c5cccc(Cl)c5)C4)CC3)c(C#N)c(=O)n(C)c2c1]

To a stirred solution 55 mg 4-(2,8-diazaspiro[4.5]decan-8-yl)-7-[(2-methoxyethyl)-(methyl)amino]-1-methyl-2-oxo-1,2-dihydroquinoline-3-carbonitrle (134 μmol, intermediate 35) in 3 mL 1,4-dioxane were added 38 μL 1-bromo-3-chlorobenzene (320 μmol, CAS 108-37-2), 175 mg cesium carbonate (537 μmol) and 21.1 mg chloro(2-dicyclohexylphosphino-2,4,6-triisopropyl-1,1-biphenyl)[2-(2-amino-1,1-biphenyl)]palladium(II) (23.8 μmol, CAS 1310584-14-5). The mixture was stirred for 5 h at 110° C. The reaction mixture was diluted with water and ethyl acetate. The organic phase was washed with water and brine, filtered and was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 35 mg of the title compound (98% purity, 49% yield).

¹H NMR (400 MHz, DMSO-d₆) δ ppm 1.78 (br t, 4H) 1.96 (t, 2H) 3.09 (s, 3H) 3.20-3.29 (m, 5H) 3.34-3.37 (m, 2H) 3.46-3.61 (m, 9H) 3.64-3.74 (m, 2H) 6.35-6.44 (m, 1H) 6.47-6.60 (m, 3H) 6.74-6.85 (m, 1H) 7.11-7.24 (m, 1H) 7.60-7.67 (m, 1H).

LC-MS (Method 2): Rₜ=1.50 min; MS (ESIpos): m/z=520.7 [M+H]⁺

### Example 35

### 7-[(2-methoxyethyl)(methyl)amino]-1-methyl-2-oxo-4-{2-[3-(trifluoromethoxy)phenyl]-2,8-diazaspiro[4.5]decan-8-yl}-1,2-dihydroquinoline-3-carboxamide

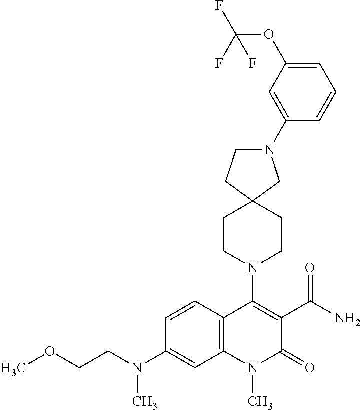 [SMILES: COCCN(C)c1ccc2c(N3CCC4(CCN(c5cccc(OC(F)(F)F)c5)C4)CC3)c(C(N)=O)c(=O)n(C)c2c1]

35 mg 7-[(2-methoxyethyl)(methyl)amino]-1-methyl-2-oxo-4-{2-[3-(trifluoromethoxy)phenyl]-2,8-diazaspiro[4.5]decan-8-yl}-1,2-dihydroquinoline-3-carbonitrle (61.4 μmol, example 32), 3.45 mg palladium(II)acetate (15.4 μmol) and 36.2 mg acetaldoxime (614 μmol) were stirred in 2.0 mL ethanol for 5 h at 80° C. The reaction mixture was added water and the mixture was extracted with ethyl acetate (2×). The organic phase was washed with brine, filtered and concentrated under reduced pressure. The residue was purified by RP-HPLC (column: X-Bridge C18 5 μm 100×30 mm, mobile phase: acetonitrile/water (0.2 vol. % ammonia 32%)-gradient) to give 30 mg of the title compound (95% purity, 79% yield).

¹H NMR (400 MHz, DMSO-d) δ ppm 1.67-1.80 (m, 4H) 1.92 (t, 2H) 3.06 (s, 3H) 3.08-3.23 (m, 6H) 3.26 (s, 3H) 3.34-3.37 (m, 2H) 3.50-3.57 (m, 5H) 3.68 (s, 2H) 6.35-6.44 (m, 2H) 6.49-6.56 (m, 2H) 6.76 (dd, 1H) 7.21-7.28 (m, 1H) 7.29-7.38 (m, 1H) 7.52-7.58 (m, 1H) 7.64-7.73 (m, 1H).

LC-MS (Method 2): Rₜ=1.44 min; MS (ESIpos): m/z=588.6 [M+H]⁺

### Example 36

### 4-[2-(3-chlorophenyl)-2,8-diazaspiro[4.5]decan-8-yl]-7-[(2-methoxyethyl)(methyl)amino]-1-methyl-2-oxo-1,2-dihydroquinoline-3-carboxamide

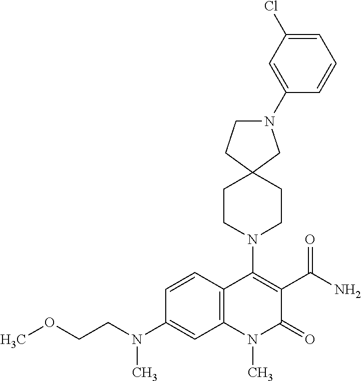 [SMILES: COCCN(C)c1ccc2c(N3CCC4(CCN(c5cccc(Cl)c5)C4)CC3)c(C(N)=O)c(=O)n(C)c2c1]

30 mg 4-[2-(3-chlorophenyl)-2,8-diazaspiro[4.5]decan-8-yl]-7-[(2-methoxyethyl)(methyl)amino]-1-methyl-2-oxo-1,2-dihydroquinoline-3-carbonitrle (57.7 μmol, example 34), 3.24 mg palladium(II)acetate (14.4 μmol) and 34.0 mg acetaldoxime (576 μmol) were stirred in 2.0 mL ethanol for 5 h at 80° C. To the reaction mixture was added water and the mixture was extracted with ethyl acetate (2×). The organic phase was washed with brine, filtered and concentrated under reduced pressure. The residue was purified by RP-HPLC (column: X-Bridge C18 5 μm 100×30 mm, mobile phase: acetonitrile/water (0.2 vol. % ammonia 32%)-gradient) to give 19 mg of the title compound (95% purity, 61% yield).

¹H NMR (400 MHz, DMSO-d₆) δ ppm 1.66-1.79 (m, 4H) 1.88-1.96 (m, 2H) 3.05 (s, 3H) 3.07-3.22 (m, 6H) 3.26 (s, 3H) 3.30-3.32 (m, 1H) 3.34-3.37 (m, 1H) 3.49-3.57 (m, 5H) 3.60-3.68 (m, 2H) 6.40-6.52 (m, 3H) 6.56-6.61 (m, 1H) 6.73-6.79 (m, 1H) 7.16 (t, 1H) 7.34 (s, 1H) 7.54 (br s, 1H) 7.68 (d, 1H).

LC-MS (Method 2): Rₜ=1.40 min; MS (ESIpos): m/z=538.6 [M+H]⁺

### EXPERIMENTAL SECTION—BIOLOGICAL ASSAYS

Human DGKα Kinase Activity Inhibition Assay.

Human DGKα inhibitory activity of compounds of the present invention was quantified employing the human DGKα kinase activity assay as described in the following paragraphs. In essence, the enzyme activity was measured by quantification of the adenosine-di-phosphate (ADP) generated as a co-product of the enzyme reaction via the “ADP-Glo™ Kinase Assay” kit from the company Promega. This detection system works as follows: In a first step the ATP not consumed in the kinase reaction is quantitatively converted to cAMP employing an adenylate cyclase (“ADP-Glo-reagent”), then the adenylate cyclase is stopped and the ADP generated in the kinase reaction converted to ATP, which subsequently generates in a luciferase-based reaction a glow-luminescence signal (“Kinase Detection Reagent”).

C-terminally FLAG-tagged, recombinant full-length human DGKα (expressed in baculovirus infected insect cells, purified using anti-Flag pulldown and size exclusion chromatography as described below, DGKa_hu_1) was used as enzyme. As substrate for the kinase 1,2-dioleoyl-sn-glycerol, reconstituted in octyl-β-D-glucopyranoside micelles, was used. For the preparation of the micelles, 1 volume of a 16.1 mM solution of 1,2-dioleoyl-sn-glycerol (Avanti, Cat. #08001-25G) in chloroform was slowly evaporated using a nitrogen stream. Subsequently, 22.55 volumes of a 510 mM solution of octyl-β-D-glucopyranoside (Sigma-Aldrich, Cat. #08001-10G) in 50 mM MOPS buffer (pH 7.4) were added, and the mixture was sonicated in an ultrasonic bath for 20 s. Then 35 volumes of 50 mM MOPS buffer (pH 7.4) were added to yield a solution of 0.28 mM 1,2 dioleoyl-sn-glycerol and 200 mM octyl-β-D-glucopyranoside, which was aliquoted, flash-frozen in liquid nitrogen, and stored at −20° C. until use. For each experiment, a fresh aliquot was quickly thawed and diluted 24-fold with aqueous assay buffer (described below) containing 95.7 μM adenosine triphosphate (Promega) to yield a 1.67-fold concentrated substrate solution.

For the assay 50 nl of a 100-fold concentrated solution of the test compound in dimethyl sulfoxide (DMSO, Sigma) was pipetted into either a white 1536-well or a white low-volume 384-well microtiter plate (both Greiner Bio-One, Frickenhausen, Germany). Subsequently, 2 μl of a solution of human DGKα in aqueous assay buffer [50 mM (3-(N-morpholino)propanesulfonic acid (MOPS, pH 7.4, Sigma-Aldrich), 1 mM dithiothreitol (DTT, Sigma-Aldrich), 100 mM NaCl (Sigma-Aldrich), 10 mM MgCl₂ (Sigma-Aldrich), 0.1% (w/v) bovine gamma globulin (BGG, Sigma-Aldrich), 1 μM CaCl₂ (Sigma-Aldrich)] were added to the wells, and the mixture was incubated for 15 min at 22° C. to allow pre-binding of the test compounds to the enzyme. The reaction was initiated by the addition of 3 μl of substrate solution [preparation described above; 11.7 μM 1,2-dioleoyl-sn-glycerol (=>final conc. in the 5 μl assay volume is 7 μM), 8.33 mM octyl-β-D-glucopyranoside (=>final conc. in 5 μl assay volume is 5 mM), and 91.67 μM adenosine triphosphate (=>final conc. in 5 μl assay volume is 55 μM) in assay buffer] and the resulting mixture was incubated for a reaction time of 20 min at 220. The concentration of DGK a was adjusted depending of the activity of the enzyme lot and was chosen appropriate to have the assay in the linear range, a typical concentration is about 0.1 nM. The reaction was stopped by the addition of 2.5 μl of “ADP-Glo-reagent” (1 to 1.5 diluted with water) and the resulting mixture was incubated at 22° C. for 1 h to convert the ATP no t consumed in the kinase reaction completely to cAMP. Subsequently 2.5 μl of the “kinase detection reagent” (1.2-fold more concentrated than recommended by the producer) were added, the resulting mixture was incubated at 22° C. for 1 h and then the luminescence measured with a suitable measurement instrument (e.g. Viewlux™ from Perkin-Elmer). The amount of emitted light was taken as a measure for the amount of ADP generated and thereby for the activity of the DGKα.

The data were normalised (enzyme reaction without inhibitor=0% inhibition, all other assay components but no enzyme=100% inhibition). Usually the test compounds were tested on the same microtiterplate in 11 different concentrations in the range of 20 M to 0.07 nM (20 M, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100-fold concentrated solutions in DMSO by serial dilutions, exact concentrations may vary depending pipettors used) in duplicate values for each concentration and IC₅₀ values were calculated using Genedata Screener™ software.

TABLE 2
IC₅₀ values of examples in in vitro human DGKa
kinase activity inhibition assays

| Example | IC₅₀ [nM] |
|---|---|
| 1 | 34 |
| 2 | 38 |
| 3 | 39 |
| 4 | 35 |
| 5 | 103 |
| 6 | 10 |
| 7 | 63 |
| 8 | 17 |
| 9 | 11 |
| 10 | 18 |
| 11 | 28 |
| 12 | 50 |
| 13 | 51 |
| 14 | 61 |
| 15 | 91 |
| 16 | 15 |
| 17 | 3.5 |
| 18 | 19 |
| 19 | 122 |
| 20 | 155 |
| 21 | 163 |
| 22 | 477 |
| 23 | 532 |
| 24 | 1700 |
| 25 | 3 |
| 26 | 42 |
| 27 | 158 |
| 28 | 1720 |
| 29 | 46 |
| 30 | 75 |
| 31 | 645 |
| 32 | 38 |
| 33 | 1170 |
| 34 | 19 |
| 35 | 68 |
| 36 | 49 |

Transactivation Assay in Jurkat IL2-Reporter Cell Line

Transactivation assays were carried out in Jurkat cells purchased from Promega (Promega, #CS187001) stably transfected with a firefly luciferase reporter gene construct under the control of the IL2-promoter. Cells were cultured as specified by the manufacturer. Bulk cells were harvested at a culture density of approx. 1E+06 cells/ml, suspended in cryo-storage medium (70% RPMI/20% FCS/10% DMSO), frozen at controlled rate of −11 min in 1.8 ml cryo-vials with cell densities of 1E+07 to 1E+08 cells per vial, and stored at −150° C. or below until further use. Frozen cells were thawed and cultured in medium at a starting density of 3.5E+05 cells/ml for 6 days. On day 6 cells were centrifuged for 5 min at 300× g, medium was decanted and cell concentration was adjusted to 5.0E+06 cells/ml with fresh assay medium (500 ml RPMI (Gibco, #22400)+5 ml L-Glutamin (Sigma, #G7513)+5 ml Penicillin/Streptomycin (Sigma #P0781)+5 ml Non-essential amino acids (Invitrogen, #11140)+5 ml sodium-pyruvate (Gibco #1136088), 5 ml FBS (Biochrom, #S0615)). Cell working stock was split in two parts: neutral control and compounds with EC30 stimulation, high control with EC100 stimulation. An antibody premix was prepared by diluting anti-CD3 (BD Pharmingen, #555329), anti-CD28 (BD Pharmingen, #555725) and goat anti mouse anti-IgG (ThermoFisher, #31160) antibodies at 1/1/4 ratio in assay medium at 2-fold of final concentration (final concentrations depend on cell batch, typically for neutral control 0.055/0.055/0.22 μg/ml, for high control 0.5/0.5/2 mg/ml). The premix solutions were added to the cells in 1+1 volume prior use.

Fifty nl of a 100-fold concentrated solution of the test compounds in DMSO were transferred into a white microtiter test plate (384, Greiner Bio-One, Germany). For this, either a Hummingbird liquid handler (Digilab, USA) or an Echo acoustic system (Labcyte, USA) was used. Five μl of the freshly prepared cell suspension was added to the wells of a test plate and incubated at 37° C. in a 5% CO₂ atmosphere. After completion of the incubation for 4 hours, 3 μl of Bio-Glo Luciferase assay reagent (Promega, #G7941, prepared as recommended by the supplier) were added to all wells. The test plate was incubated at 20° C. for 10 min before measurement of the luminescence in a microplate reader (typically Pherastar by BMG, Germany, or ViewLux by Perkin-Elmer, USA). Data were normalized (neutral control=0% effect, high control=100% effect). Compounds were tested in duplicates at up to 11 concentrations (typically 20 μM, 5.7 μM, 1.6 μM, 0.47 μM, 0.13 μM, 38 nM, 11 nM, 3.1 nM, 0.89 nM, 0.25 nM and 0,073 nM). Dilution series were made prior to the assay in a 100-fold concentrated form by serial dilution. EC₅₀ values were calculated by 4-Parameter fitting using a commercial software package (Genedata Analyzer, Switzerland).

Polyclonal Activation of Human PBMCs

To test the effect of DGKα compounds on IL-2 and IFN-γ secretion of human Peripheral Blood Mononuclear Cells (PBMCs) a 24h human PBMC assay is performed as screening assay. For this, a 96 well flat bottom plate is coated with a suboptimal stimulation condition (EC 10-30) of human aCD3 (Invitrogen, clone OKT3) antibody in 50 μl PBS/well at 4° C. overnight. PBMCs isolated and frozen at liquid N₂ from leucapherese samples is thawed and resuspended in culture medium (X-Vivo-20). 4×10⁵ cells/well are plated. Wells are treated with the respective compound concentrations (5-fold dilution steps from 10 μM to 3 nM) and the final DMSO concentration per well is 0.1%. Medium+DMSO (0.1%) is used as baseline value. As positive controls 1000 ng/ml aCD3+aCD28 (1 μg/ml) and a DGKα reference compound is used. After 24 h the medium is collected and hIL-2 or hIFN-γ ELISA are performed. The following parameters are calculated: EC₅₀ value, concentration at 50% increase; max increase in % and respective concentration and maximum effect normalized to max concentration (10 μM) of a selected DGKα reference compound.

In Vitro Activation of Mouse OT-I Antigen-Specific T-Cells

To test the effect of DGKα compounds in murine antigen-specific T-cells, spleens and lymph nodes of OT-I mice are collected and mashed through a 40 μm cell strainer and incubated for 1 min in 1 ml ACK lysing buffer (Gibco)/spleen. 4×106 cells/ml are incubated in medium containing 0.05 ng/ml SIINFEKL in a 50 ml falcon at 37° C. for 30 min. Afterwards cells are centrifuged and 4×106 cells/ml are resuspended in fresh medium (DMEM; 10% FCS, 1% Pen/Strep, 0.1% s-mercaptoethanol, 1% HEPES). 4×105 cells are plated per well in a 96-well round bottom plate. Wells are treated with respective compound concentrations (5-fold dilution steps from 10 μM to 3 nM) in a final DMSO concentration of 0.1%. Medium+DMSO (0.1%) is used as baseline value. As positive controls cells incubated with the 4×SIINFEKL concentration (0.2 ng/ml) and a DGKa reference compound are used. The plates are centrifuged to reduce the distance between T-cells and APCs before incubation. After 24 h the medium is collected and mIL-2 or mIFN-γ ELISAs are performed. The following parameters are calculated: EC₅₀ value, concentration at 50% increase; max increase in % and respective concentration and maximum effect normalized to max concentration (10 μM) of a selected DGKα reference compound.

DGKα Surface Plasmon Resonance Interaction Assay

The ability of the compounds described in this invention to bind to DGKα may be determined using surface plasmon resonance (SPR). This allows for the quantification of binding in terms of the equilibrium dissociation constant (K_{D} [M]), as well as association and dissociation rate constants (kₒₙ [1/Ms] and k_{off} [1/s], respectively). The measurements may be performed using Biacore® T200, Biacore® S200 or Biacore® 8K (GE Healthcare).

All buffers described in this section were prepared with 10×HBS-P+ Buffer (GE Healthcare, #BR100671) supplemented with additional buffer components as indicated below, dithiothreitol (DTT from Sigma, #D0632-25G), Adenosine 5′-triphosphate (ATP from Sigma, #A26209-10G), MgCl₂ (Sigma, #M1028-100ML), dimethyl sulfoxide (DMSO from Biomol, #54686.500).

For SPR measurements, recombinant and biotinylated human DGKα (DGKa_hu_1Avi) was immobilized via the streptavidin-biotin interaction onto a Series S Sensor Chip SA (GE Healthcare, #BR-1005-31). Briefly, DGKα was diluted to a concentration of 19 μg/ml in Immobilization Buffer (10 mM HEPES, 150 mM NaCl, 0.05% v/v Surfactant P20, 2 mM MgCl₂, 1 mM DTT, pH 7.4) and captured on the SA Chip surface using a flow rate of 10 μl/min for 500 seconds at a temperature of 10° C. Immobilization levels of approximately 8000-10000 RU were typically achieved. The reference surface consisted of a streptavidin surface without immobilized protein. Compounds were diluted from 10 mM DMSO stock solution into Running Buffer (10 mM HEPES, 150 mM NaCl, 0.05% v/v Surfactant P20, 2 mM MgCl₂, 1 mM DTT, 0.2 mM ATP and 1% v/v DMSO, pH 7.4). For SPR-binding measurements serial dilutions (typically 1:3 dilutions resulting in 8 concentrations up to 2 μM or 20 μM) were injected over immobilized protein. Binding affinity and kinetics were measured at 18° C. and at a flow rate of 100 μl/min.

For regeneration of slowly dissociating compounds an additional regeneration step was included by injection of Regeneration Buffer without ATP (10 mM HEPES, 150 mM NaCl, 0.05% v/v Surfactant P20, 1 mM DTT and 1% v/v DMSO, pH 7.4) for 200 s at a flow rate of 30 μl/min

The double-referenced sensorgrams were fit to a simple reversible Langmuir 1:1 reaction mechanism as implemented in the Biacore® T200, S200 and 8K evaluation software (Biacore T200 Evaluation Software version 2.0, Biacore S200 Evaluation Software version 1.0, Biacore 8K Evaluation Software v 1.1.1.7442, GE Healthcare).

Expression of DGKα in Insect Cells Using the Baculovirus System

Expression Constructs:

The cDNA encoding the full length sequence of human DGKα (Uniprot P23743) was optimized for expression in eukaryotic cells and synthesized by the GeneArt Technology at Life Technologies.

The DNA Sequence Encoded the Following Sequence:

Construct DGKa_hu Amino Acid M1 to S735

Additionally the expression construct encoded: a Kozak DNA sequence for translation initiation (GCCACC), at the C-terminus a Flag (DYKDDDDK) sequence followed by two stop codons and additionally 5′ and 3′ att-DNA sequences for Gateway Cloning.

The DGKα construct was subcloned using the Gateway Technology into the Destination vector pD-INS. The vector pD-INS is a Baculovirus transfer vector (based on vector pVL1393, Pharmingen) which enables the expression of the DGK-Flag protein. The respective protein was named DNA_hu_1.

Additionally the DNA construct DGKa_hu with C-terminal Flag tag was also subcloned in to the Destination vector pD-INSA. This Baculovirus transfer vector is designed to fuse a His6 tag+Avi tag protein sequence to N-terminus of the DGKa_hu-Flag protein. The complete encoded protein was designated DGKa_hu_1Avi. The Avi-tag sequence enables a site-specific in-vitro biotinylation of the DGKα protein.

Generation of Recombinant Baculovirus

In separate approaches each of the two DGK transfer vectors was co-transfected in Sf9 cells with Baculovirus DNA (Flashbac Gold DNA, Oxford Expression Technologies) using Fugene HD (Roche). After 5 days the supernatant of the transfected cells containing the recombinant Baculovirus encoding the various DGK proteins was used for further infection of Sf9 cells for virus amplification whereby the virus titer was monitored using qPCR.

DGK Expression in Sf9 Cells Using Bioreactor

Sf9 cells cultured (Insect-xpress medium, Lonza, 27° C.) in a Wave-bioreactor with a disposable culture bag were infected at a cell density of 10⁶ cells/mL with one of the recombinant baculovirus stocks at a multiplicity of infection of 1 and incubated for 72. Subsequently the cells were harvested by centrifugation (800×g) and cell pellet frozen at −80° C.

To produce biotinylated DGKa_hu_1Avi the Sf9 cells in the bioreactor were co-infected with the Baculovirus encoding DGKa_hu_1Avi as well as with a Baculovirus encoding the biotinylation enzyme BirA.

Purification of the DGK-Flag Proteins:

Purification of the DGK-Flag proteins was achieved by a two-step chromatography procedure as follows.

The pelleted cells (from 8 L cell culture) were resuspended in Lysis-Buffer (50 mM Tris HCl 7.4; 150 mM NaCl; 10 mM MgCl₂; 1 μM CaCl₂; 1 mM DTT; 0.1% NP-40; 0.1% NP-40; Complete Protease Inhibitor Cocktail-(Roche)) and lysed by a freeze-thaw cycle followed by an incubation on ice for 60 min. The lysate was centrifuged at 63.000×g for 30 min. at 4° C. The soluble supernatant was than incubated with 25 mL anti-Flag M2 Agarose (Sigma) in a plastic flask rotating for 16 h at 4° C. for binding of the DGK-FI ag proteins, subsequently rinsed with 10×25 mL Wash-Buffer (50 mM Tris HCl 7.4; 150 mM NaCl; 10 mM MgCl₂; 1 μM CaCl₂; 1 mM DTT) and finally the bound protein was eluted using Elusion-Buffer (Wash-Buffer with 300 μg/mL FLAG-Peptide, incubated 30 min. at 4° C. with 3×15 mL).

The elution fractions from the affinity chromatography were concentrated (using Amicon Ultra 15, Centrifugal Filters, 30 kDa MW cut-off; Millipore #UFC903024) to 10 mL and applied to a size exclusion chromatography column (S200 prep grade 26/60, GE Healthcare) and the resulting monomeric peak fraction was collected, pooled and again concentrated. Wash-buffer was used for size exclusion chromatography and the final concentrated sample. The final protein sample concentration was 5-10 mg/mL and the yield was 1-2 mg final protein per L cell culture. For DGKa_hu_1Avi a biotinylation level of 100% was demonstrated by mass spectrometry.

In Vivo Activation of Murine Antigen Specific OT1 T Cells

Oral Administration of compounds enhances antigen-specific T cell activation in vivo.

Direct detection of antigen-specific T cell proliferation in vivo is technically challenging, since it requires the presence of T cells specific for a cognate antigen and also a specific measurement procedure for cell proliferation. Both these requirements are fulfilled in the OT-1 transfer model, which utilizes the direct transfer of CD8 T cells transgenic for a T cell receptor recognizing an Ovalbumin-derived peptide as antigen. Before transfer, these cells are labeled with the fluorescent dye CFSE, which is diluted by every cell division and therefore allows detection of cell proliferation. After transfer of the CFSE-labeled T cells, mice are vaccinated with the Ovalbumin antigen OVA-30. Only transferred OT-1 cells are able to recognize the OVA-antigen presented by APC and only these transferred T cells then get activated. Flow cytometric analysis of CFSE-levels in the OT-1 cells can be combined with measurement of multiple activation markers like CD69, CD25 and PD1.

In particular, mice receive 2×10×6 CFSE-labeled OT-1 T cells and are vaccinated one day later by intravenous application of 2.5 μg OVA-30. Mice are then divided into groups which receive vehicle only, compound alone or in combination with other immune modulating agents. Mice are treated for 2 to 20 days and T cell composition (incl. transferred OT-1 cells) of spleen, blood and lymphodes are analysed by FACS.

In Vivo Syngeneic Tumor Models

Animals are assigned to a study at the age of 6-8 weeks. Animal husbandry, feeding and health conditions are according to animal welfare guidelines. Syngeneic tumor cell lines are cultivated with appropriate medium and splitted at least 3 times before inoculation. Female mice are inoculated with appropriate amount of tumor cells in medium or a medium/matrigel mixture s.c, i.v. or i.p depending on the model. After 4-10 days the animals are randomized and therapeutic treatment starts when tumors reach a size of approx. 40-70 mm2.

Tumor size is measured using calipers determining length (a) and width (b). Tumor volume is calculated according to:

v=(a×b\hat{ }2)/2

Significance of monotherapies and combination treatment is calculated versus control group as determined by 2-Way ANOVA analysis.

1: A compound of formula (I): 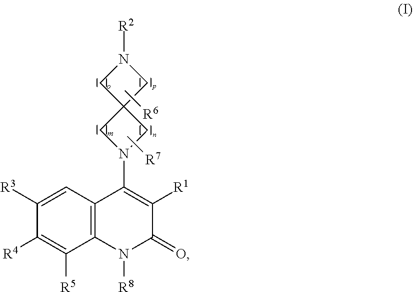
wherein:
R¹ is selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² is selected from the group consisting of phenyl, naphthyl, and 5- to 10-membered heteroaryl,
wherein phenyl, naphthyl, and 5- to 10-membered heteroaryl are optionally substituted, one, two, three, or four times, wherein each substituent is independently selected from the group consisting of a halogen atom, C₁-C₆-alkyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
or two substituents of said phenyl group, when they are attached to adjacent ring atoms, are linked to one another in such a way that they jointly form a group selected from the group consisting of —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O—, and —O—(CH₂)₂—O—,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl and C₁-C₆-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₆-cycloalkyl group is optionally substituted, one or two times, wherein each substituent is independently a halogen atom or a C₁-C₄-alkyl group,
wherein said phenyl, phenoxy, and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl, wherein said 4- to 7-membered heterocycloalkyl group and
5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy group are optionally substituted with a group selected from C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl and —N(R⁹)(R¹⁰),
wherein the C₃-C₄-cycloalkyl group is optionally substituted, one or two times, wherein each substituent independently selected from the group consisting of a halogen atom, cyano, and hydroxy,
wherein said C₃-C₆-cycloalkyl and C₄-C₆-cycloalkenyl groups are optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁴ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, N(R⁹)(R¹⁰), N(R¹⁶)(R¹⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy group are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl and —N(R⁹)(R¹⁰),
wherein the C₃-C₄-cycloalkyl group is optionally substituted, one or two times, wherein each substituent is independently selected from h group consisting of a halogen atom,
cyano, and hydroxy,
wherein said C₃-C₆-cycloalkyl and C₄-C₆-cycloalkenyl groups are optionally substituted, one or two times, wherein each substituent is independently a halogen atom or a C₁-C₄-alkyl group,
wherein said phenyl and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁵ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group gr 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group aa optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₆-cycloalkyl and C₄-C₆-cycloalkenyl groups are optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁶ is a hydrogen atom, or a fluorine atom or a C₁-C₄-alkyl group;
R⁷ is a hydrogen atom, or a fluorine atom or a C₁-C₄-alkyl group;
R⁸ is methyl or ethyl;
R⁹ and R¹⁰ are each independently selected from the group consisting of a hydrogen atom, C₁-C₄-alkyl, (C₁-C₄-alkoxy)-(C₂-C₄-alkyl)-, C₃-C₄-cycloalkyl, and C₂-C₄-haloalkyl, or
R⁹ and R¹⁰ together with the nitrogen to which they are attached form a nitrogen containing 4- to 7-membered heterocycloalkyl group,
wherein said nitrogen containing 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, form a 4- to 7-membered heterocycloalkyl group,
wherein said 4- to 7-membered heterocycloalkyl group is optionally substituted, one or two times, wherein each substituent is independently selected from the group consisting of a halogen atom, C₁-C₄-alkyl, C₃-C₄-cycloalkyl, C₁-C₄-haloalkyl, hydroxy, and oxo;
R¹¹ represents is selected from the group consisting of a hydrogen atom, C₁-C₄-alkyl, C₁-C₄-hydroxyalkyl, C₁-C₄-haloalkyl, phenyl, and 5- or 6-membered heteroaryl,
wherein said phenyl group and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹² is a hydrogen atom or a C₁-C₄-alkyl group;
R¹³ is selected from the group consisting of a hydrogen atom, C₁-C₆-alkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁴ is a selected from the group consisting of C₁-C₆-alkyl, C₁-C₆-haloalkyl, C₃-C₆-cycloalkyl, phenyl, and 5- or 6-membered heteroaryl,
wherein said phenyl group and 5- or 6-membered heteroaryl group is optionally substituted, one or two times, wherein each substituent is independently selected from a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁵ is a hydrogen atom or a C₁-C₄-alkyl group;
R¹⁶ is selected from the group consisting of a hydrogen atom, C₁-C₄-alkyl, C₃-C₄-cycloalkyl, and C₂-C₄-haloalkyl;
R¹⁷ is a 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, C₁-C₄-alkoxy, hydroxy, and oxo,
wherein said 4- to 7-membered heterocycloalkyl group is connected to the rest of the molecule via a carbon atom of the 4- to 7-membered heterocycloalkyl group; and
m, n, o, and p are independently selected from the group consisting of 1, 2, and 3,
or a stereoisomer, a tautomer, an N-oxide, a hydrate, a solvate, or a salt thereof, or a mixture of any of the foregoing.

2: The compound according to claim 1, wherein:
R¹ is selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² is selected from the group consisting of phenyl, naphthyl, and 5- to 10-membered heteroaryl,
wherein phenyl, naphthyl, and 5- to 10-membered heteroaryl group are optionally substituted, one, two, three, or four times, wherein each substituent is independently selected from the group consisting of a halogen atom, C₁-C₄-alkyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
or two substituents of said phenyl group, when they are attached to adjacent ring atoms, are linked to one another in such a way that they jointly form a group selected from the group consisting of —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂—O—CH₂—, —O—CH₂—O—, and —O—(CH₂)₂—O—,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl and C₁-C₄-alkoxy groups are optionally substituted with a group selected from C₃-C₄-cycloalkyl, phenyl, and 4- to 7-membered heterocycloalkyl,
wherein said 4- to 7-membered heterocycloalkyl group,
is connected to the rest of the molecule via a carbon atom of said 4-to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl group is optionally substituted, one or two times, wherein each substituent is independently a halogen atom or a C₁-C₄-alkyl group,
wherein said phenyl, phenoxy, and 5- or 6-membered heteroaryl group aa optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and
5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl, C₂-C₄-alkenyl, C₂-C₄-alkynyl, and C₁-C₄-alkoxy group is optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁴ is selected from the group consisting of a hydrogen atom, or a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, N(R⁹)(R¹⁰), N(R⁶)(R⁷), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl, C₂-C₄-alkenyl, C₂-C₄-alkynyl, and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁵ is selected from the group consisting of a hydrogen atom, a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, S(═O)₂R¹⁴, cyano, hydroxy, N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group gr 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R⁰), and oxo,
wherein said C₁-C₄-alkyl, C₂-C₄-alkenyl, C₂-C₄-alkynyl, and C₁-C₄-alkoxy groups are optionally substituted with a group selected from C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁶ is a hydrogen atom, a fluorine atom, or a C₁-C₄-alkyl group;
R⁷ is a hydrogen atom, a fluorine atom, or a C₁-C₄-alkyl group;
R⁸ is methyl or ethyl;
R⁹ and R¹⁰ are each independently selected from the group consisting of a hydrogen atom, C₁-C₄-alkyl, (C₁-C₄-alkoxy)-(C₂-C₄-alkyl)-, C₃-C₄-cycloalkyl and C₂-C₄-haloalkyl, or
R⁹ and R¹⁰ together with the nitrogen to which they are attached form a nitrogen containing 4- to 7-membered heterocycloalkyl group,
wherein said nitrogen containing 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, form a 4- to 7-membered heterocycloalkyl group,
wherein said 4- to 7-membered heterocycloalkyl group is optionally substituted, one or two times, wherein each substituent is independently selected from the group consisting of a halogen atom, C₁-C₄-alkyl, C₃-C₄-cycloalkyl, C₁-C₄-haloalkyl, hydroxy, and oxo;
R¹¹ is selected from the group consisting of a hydrogen atom, C₁-C₄-alkyl, C₁-C₄-hydroxyalkyl, C₁-C₄-haloalkyl, phenyl, and 5- or 6-membered heteroaryl,
wherein said phenyl group and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹² is a hydrogen atom or a C₁-C₄-alkyl group;
R¹³ is selected from the group consisting of a hydrogen atom, C₁-C₄-alkyl, phenyl, and 5- or 6-membered heteroaryl,
wherein said phenyl group and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁴ is a group selected from the group consisting of C₁-C₄-alkyl, C₁-C₄-haloalkyl, C₃-C₅-cycloalkyl, phenyl, and 5- or 6-membered heteroaryl,
wherein said phenyl group and 5- or 6-membered heteroaryl group arg optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁵ is a hydrogen atom or a C₁-C₄-alkyl group;
R¹⁶ is selected from the group consisting of a hydrogen atom, C₁-C₄-alkyl, C₃-C₄-cycloalkyl, and C₂-C₄-haloalkyl;
R¹⁷ is a 4- to 7-membered heterocycloalkyl group,
wherein said 4- to 7-membered heterocycloalkyl group is optionally substituted, one, two or three times, wherein each substituent is independently selected from the group consisting of a halogen atom, C₁-C₄-alkyl, C₃-C₄-cycloalkyl, C₁-C₄-alkoxy, hydroxy, and oxo,
wherein said 4- to 7-membered heterocycloalkyl group is connected to the rest of the molecule via a carbon atom of the 4- to 7-membered heterocycloalkyl group; and
m, n, o, and p are each independently selected from the group consisting of 1, 2, and 3,
or a stereoisomer, a tautomer, an N-oxide, a hydrate, a solvate, or a salt thereof, or a mixture of any of the foregoing.

3: The compound according to claim 1, wherein:
R¹ selected from is selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² selected from the group consisting of phenyl, naphthyl, and 5- to 10-membered heteroaryl,
wherein phenyl, naphthyl, and 5- to 10-membered heteroaryl group is optionally substituted, one, two, three, or four times, wherein each substituent is independently selected from the group consisting of a halogen atom, C₁-C₄-alkyl, C₁-C₄-haloalkyl, (C₁-C₂-alkoxy)-(C₁-C₄-alkyl)-, C₁-C₄-alkoxy, C₁-C₄-haloalkoxy, and —N(R⁹)(R¹⁰),
wherein said C₁-C₄-alkyl and C₁-C₄-alkoxy groups are optionally substituted with a group selected from C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy;
R³ is selected from the group consisting of a hydrogen atom, a halogen atom, C₁-C₄-alkyl, C₁-C₄-haloalkyl, (C₁-C₂-alkoxy)-(C₁-C₄-alkyl)-, C₁-C₄-alkoxy, and —P(═O)(R¹⁴)₂,
wherein said C₁-C₄-alkyl and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy;
R⁴ is selected from the group consisting of a hydrogen atom, a halogen atom, C₁-C₄-alkyl, C₁-C₄-haloalkyl, (C₁-C₂-alkoxy)-(C₁-C₄-alkyl)-, C₁-C₄-alkoxy, N(R⁹)(R¹⁰), N(R¹⁶)(R¹⁷), —P(═O)(R¹⁴)₂, and (4- to 7-membered heterocycloalkyl)oxy,
wherein said (4- to 7-membered heterocycloalkyl)oxy group is optionally substituted, one or two times, wherein each substituent is independently selected from the group consisting of a halogen atom, C₁-C₂-alkyl, C₁-C₂-haloalkyl, C₁-C₂-alkoxy, C₃-C₄-cycloalkyl, and oxo,
wherein said C₁-C₄-alkyl and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹¹), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy;
R⁵ is selected from the group consisting of a hydrogen atom, a halogen atom, C₁-C₄-alkyl, C₁-C₄-haloalkyl, (C₁-C₂-alkoxy)-(C₁-C₄-alkyl)-, C₁-C₄-alkoxy, N(R⁹)(R¹⁰), and —P(═O)(R¹⁴)₂,
wherein said C₁-C₄-alkyl and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy;
R⁶ is a hydrogen atom;
R⁷ is a hydrogen atom;
R⁸ is methyl or ethyl;
R⁹ and R¹⁰ are independently selected from the group consisting of a hydrogen atom, C₁-C₄-alkyl, (C₁-C₄-alkoxy)-(C₂-C₄-alkyl)-, C₃-C₄-cycloalkyl, and C₂-C₄-haloalkyl, or
R⁹ and R¹⁰ together with the nitrogen to which they are attached form a nitrogen containing 4- to 7-membered heterocycloalkyl group,
wherein said nitrogen containing 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, form a 4- to 7-membered heterocycloalkyl group,
wherein said 4- to 7-membered heterocycloalkyl group is optionally substituted, one or two times, wherein each substituent is independently selected from the group consisting of a halogen atom, C₁-C₄-alkyl, C₃-C₄-cycloalkyl, C₁-C₄-haloalkyl, hydroxy, and oxo;
R¹⁴ is selected from the group consisting of C₁-C₄-alkyl, C₁-C₄-haloalkyl, C₃-C₅-cycloalkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁵ is a hydrogen atom or a C₁-C₄-alkyl group;
R¹⁶ is selected from the group consisting of a hydrogen atom, C₁-C₄-alkyl, C₃-C₄-cycloalkyl, and C₂-C₄-haloalkyl;
R¹⁷ is a 4- to 7-membered heterocycloalkyl group,
wherein said 4- to 7-membered heterocycloalkyl group is optionally substituted, one or two times, with a C₁-C₄-alkyl group,
wherein said 4- to 7-membered heterocycloalkyl group is connected to the rest of the molecule via a carbon atom of the 4- to 7-membered heterocycloalkyl group; and
m, n, o, and p are each independently selected from the group consisting of 1, 2, and 3,
or a stereoisomer, a tautomer, an N-oxide, a hydrate, a solvate, or a salt thereof, or a mixture of any of the foregoing.

4: The compound according to claim 1, wherein:
R¹ is selected from the group consisting of cyano, —C(═O)NH₂, —C(═O)N(H)CH₃, and —C(═O)N(CH₃)₂;
R² is a phenyl group,
wherein the phenyl group is optionally substituted, one, two, three, or four times, wherein each substituent is independently selected from the group consisting of a halogen atom, C₁-C₄-alkoxy, C₁-C₄-haloalkoxy, and —N(R⁹)(R¹⁰);
R³ is a hydrogen atom, a halogen atom, or a —P(═O)(R¹⁴)₂ group;
R⁴ is selected from the group consisting of a hydrogen atom, a halogen atom, N(R⁹)(R¹⁰), N(R¹⁶)(R¹⁷), —P(═O)(R¹⁴)₂, and (4- to 7-membered heterocycloalkyl)oxy,
wherein said (4- to 7-membered heterocycloalkyl)oxy group is optionally substituted, one or two times, wherein each substituent is independently selected from the group consisting of a halogen atom, C₁-C₂-alkyl, C₁-C₂-haloalkyl, C₁-C₂-alkoxy, C₃-C₄-cycloalkyl, and oxo;
R⁵ is a hydrogen atom, a halogen atom, or a —P(═O)(R¹⁴)₂ group;
R⁶ is a hydrogen atom;
R⁷ is a hydrogen atom;
R⁸ is methyl or ethyl;
R⁹ and R¹⁰ are independently selected from the group consisting of a hydrogen atom, C₁-C₄-alkyl, and (C₁-C₄-alkoxy)-(C₂-C₄-alkyl)-,
or
R⁹ and R¹⁰ together with the nitrogen to which they are attached form a nitrogen containing 4- to 7-membered heterocycloalkyl group,
wherein said nitrogen containing 4- to 7-membered heterocycloalkyl group is optionally substituted, one, two, or three times, wherein each substituent is independently selected from the group consisting of a halogen atom, C₁-C₄-alkyl, and oxo;
R¹⁴ is selected from the group consisting of C₁-C₄-alkyl, C₁-C₄-haloalkyl, C₃-C₅-cycloalkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁶ is selected from the group consisting of a hydrogen atom, C₁-C₄-alkyl, C₃-C₄-cycloalkyl, and C₂-C₄-haloalkyl;
R¹⁷ is a 4- to 7-membered heterocycloalkyl group,
wherein said 4- to 7-membered heterocycloalkyl group is optionally substituted, one or two times, with a C₁-C₄-alkyl group,
wherein said 4- to 7-membered heterocycloalkyl group is connected to the rest of the molecule via a carbon atom of the 4- to 7-membered heterocycloalkyl group;
m, n, o, and p are independently selected from the group consisting of 1, 2, and 3,
or a stereoisomer, a tautomer, an N-oxide, a hydrate, a solvate, or a salt thereof, or a mixture of any of the foregoing.

5: The compound according to claim 1, wherein:
R¹ is cyano or —C(═O)NH₂;
R² is a phenyl group,
wherein the phenyl group is optionally substituted, one, two, three, or four times, wherein each substituent is independently selected from the group consisting of a halogen atom, C₁-C₄-alkoxy, C₁-C₄-haloalkoxy, and —N(R⁹)(R¹⁰);
R³ is a hydrogen atom,
R⁴ is a hydrogen atom, a halogen atom, N(R⁹)(R¹⁰), N(R¹⁶)(R¹⁷), and (4- to 7-membered heterocycloalkyl)oxy;
R⁵ is a hydrogen atom;
R⁶ is a hydrogen atom;
R⁷ is a hydrogen atom;
R⁸ is a methyl group;
R⁹ and R¹⁰ are each independently selected from the group consisting of a hydrogen atom, C₁-C₄-alkyl, and (C₁-C₄-alkoxy)-(C₂-C₄-alkyl)-,
or
R⁹ and R¹⁰ together with the nitrogen to which they are attached form a nitrogen containing 4- to 7-membered heterocycloalkyl group,
wherein said nitrogen containing 4- to 7-membered heterocycloalkyl group is optionally substituted, one, two, or three times, wherein each substituent is independently selected from the group consisting of a halogen atom, C₁-C₄-alkyl, and oxo;
R¹⁶ is a hydrogen atom or a C₁-C₄-alkyl group,
R¹⁷ is a 4- to 7-membered heterocycloalkyl group,
wherein said 4- to 7-membered heterocycloalkyl group is connected to the rest of the molecule via a carbon atom of the 4- to 7-membered heterocycloalkyl group,
m, n, o, and p are each independently selected from the group consisting of 1, 2, and 3,
or a stereoisomer, a tautomer, an N-oxide, a hydrate, a solvate, or a salt thereof, or a mixture of any of the foregoing.

6: The compound according to claim 1, wherein:
R¹ is cyano or —C(═O)NH₂;
R² is a phenyl group,
wherein the phenyl group is optionally substituted, one or two times, wherein each substituent is independently selected from the group consisting of a fluorine atom, a chlorine atom, methoxy, trifluoromethoxy, morpholin-4-yl, N,N-dimethylamino, and 2-oxopyrrolidin-1-yl;
R³ is a hydrogen atom;
R⁴ is selected from the group consisting of a hydrogen atom, a bromine atom, 4-methylpiperazin-1-yl, (2-methoxyethyl)(methyl)amino, methyl(tetrahydrofuran-3-yl)amino, (tetrahydrofuran-3-yl)oxy, (tetrahydro-2H-pyran-3-yl)oxy, and (tetrahydro-2H-pyran-4-yl)oxy;
R⁵ is a hydrogen atom;
R⁶ is a hydrogen atom;
R⁷ is a hydrogen atom;
R⁸ is a methyl group;
m and n are each independently 1 or 2;
o is 1;
p is selected from the group consisting of 1, 2, and 3,
or a stereoisomer, a tautomer, an N-oxide, a hydrate, a solvate, or a salt thereof, or a mixture of any of the foregoing.

7: The compound according to claim 1, wherein the compound is selected from the group consisting of:
1-methyl-2-oxo-4-(2-phenyl-2,8-diazaspiro[4.5]decan-8-yl)-1,2-dihydroquinoline-3-carbonitrile;
4-[2-(4-fluorophenyl)-2,8-diazaspiro[4.5]decan-8-yl]-1-methyl-2-oxo-1,2-dihydroquinoline-3-carbonitrile;
4-[2-(3,4-difluorophenyl)-2,8-diazaspiro[4.5]decan-8-yl]-1-methyl-2-oxo-1,2-dihydroquinoline-3-carbonitrile;
4-[2-(4-fluorophenyl)-2,6-diazaspiro[3.4]octan-6-yl]-1-methyl-2-oxo-1,2-dihydroquinoline-3-carbonitrile;
4-[2-(4-methoxyphenyl)-2,6-diazaspiro[3.4]octan-6-yl]-1-methyl-2-oxo-1,2-dihydroquinoline-3-carbonitrile;
4-[2-(4-chlorophenyl)-2,6-diazaspiro[3.4]octan-6-yl]-1-methyl-2-oxo-1,2-dihydroquinoline-3-carbonitrile;
4-[6-(4-fluorophenyl)-2,6-diazaspiro[3.4]octan-2-yl]-1-methyl-2-oxo-1,2-dihydroquinoline-3-carbonitrile;
1-methyl-2-oxo-4-{2-[4-(trifluoromethoxy)phenyl]-2,8-diazaspiro[4.5]decan-8-yl}-1,2-dihydroquinoline-3-carbonitrile;
4-[2-(3-methoxyphenyl)-2,8-diazaspiro[4.5]decan-8-yl]-1-methyl-2-oxo-1,2-dihydroquinoline-3-carbonitrile;
4-[2-(2-methoxyphenyl)-2,8-diazaspiro[4.5]decan-8-yl]-1-methyl-2-oxo-1,2-dihydroquinoline-3-carbonitrile;
4-{2-[3-(dimethylamino)phenyl]-2,8-diazaspiro[4.5]decan-8-yl}-1-methyl-2-oxo-1,2-dihydroquinoline-3-carbonitrile;
1-methyl-2-oxo-4-{2-[3-(trifluoromethoxy)phenyl]-2,8-diazaspiro[4.5]decan-8-yl}-1,2-dihydroquinoline-3-carbonitrile;
4-{2-[2-(dimethylamino)phenyl]-2,8-diazaspiro[4.5]decan-8-yl}-1-methyl-2-oxo-1,2-dihydroquinoline-3-carbonitrile;
1-methyl-2-oxo-4-{2-[2-(trifluoromethoxy)phenyl]-2,8-diazaspiro[4.5]decan-8-yl}-1,2-dihydroquinoline-3-carbonitrile;
4-[2-(4-methoxyphenyl)-2,8-diazaspiro[4.5]decan-8-yl]-1-methyl-2-oxo-1,2-dihydroquinoline-3-carbonitrile;
4-[6-(4-fluorophenyl)-2,6-diazaspiro[3.5]nonan-2-yl]-1-methyl-2-oxo-1,2-dihydroquinoline-3-carbonitrile;
1-methyl-2-oxo-4-{2-[4-(trifluoromethoxy)phenyl]-2,6-diazaspiro[3.4]octan-6-yl}-1,2-dihydroquinoline-3-carbonitrile;
1-methyl-2-oxo-4-{6-[4-(trifluoromethoxy)phenyl]-2,6-diazaspiro[3.4]octan-2-yl}-1,2-dihydroquinoline-3-carbonitrile;
1-methyl-2-oxo-4-{7-[4-(trifluoromethoxy)phenyl]-2,7-diazaspiro[4.4]nonan-2-yl}-1,2-dihydroquinoline-3-carbonitrile;
4-[2-(3-methoxyphenyl)-2,6-diazaspiro[3.4]octan-6-yl]-1-methyl-2-oxo-1,2-dihydroquinoline-3-carbonitrile;
4-{2-[4-(dimethylamino)phenyl]-2,6-diazaspiro[3.4]octan-6-yl}-1-methyl-2-oxo-1,2-dihydroquinoline-3-carbonitrile;
1-methyl-2-oxo-4-{2-[4-(2-oxopyrrolidin-1-yl)phenyl]-2,6-diazaspiro[3.4]octan-6-yl}-1,2-dihydroquinoline-3-carbonitrile;
4-[7-(4-fluorophenyl)-2,7-diazaspiro[4.4]nonan-2-yl]-1-methyl-2-oxo-1,2-dihydroquinoline-3-carbonitrile;
1-methyl-4-{2-[4-(morpholin-4-yl)phenyl]-2,6-diazaspiro[3.4]octan-6-yl}-2-oxo-1,2-dihydroquinoline-3-carbonitrile;
1-methyl-2-oxo-4-{2-[4-(trifluoromethoxy)phenyl]-2,8-diazaspiro[4.5]decan-8-yl}-1,2-dihydroquinoline-3-carboxamide;
4-[2-(4-chlorophenyl)-2,6-diazaspiro[3.4]octan-6-yl]-1-methyl-2-oxo-1,2-dihydroquinoline-3-carboxamide;
4-[2-(4-fluorophenyl)-2,6-diazaspiro[3.4]octan-6-yl]-1-methyl-2-oxo-1,2-dihydroquinoline-3-carboxamide;
4-{2-[4-(dimethylamino)phenyl]-2,6-diazaspiro[3.4]octan-6-yl}-1-methyl-2-oxo-1,2-dihydroquinoline-3-carboxamide;
7-bromo-1-methyl-2-oxo-4-{2-[4-(trifluoromethoxy)phenyl]-2,6-diazaspiro[3.4]octan-6-yl}-1,2-dihydroquinoline-3-carbonitrile;
7-bromo-4-[2-(4-chlorophenyl)-2,6-diazaspiro[3.4]octan-6-yl]-1-methyl-2-oxo-1,2-dihydroquinoline-3-carbonitrile;
1-methyl-7-(4-methylpiperazin-1-yl)-2-oxo-4-{2-[3-(trifluoromethoxy)phenyl]-2,8-diazaspiro[4.5]decan-8-yl}-1,2-dihydroquinoline-3-carbonitrile;
7-[(2-methoxyethyl)(methyl)amino]-1-methyl-2-oxo-4-{2-[3-(trifluoromethoxy)phenyl]-2,8-diazaspiro[4.5]decan-8-yl}-1,2-dihydroquinoline-3-carbonitrile;
4-[2-(3-chlorophenyl)-2,8-diazaspiro[4.5]decan-8-yl]-1-methyl-7-(4-methylpiperazin-1-yl)-2-oxo-1,2-dihydroquinoline-3-carbonitrile;
4-[2-(3-chlorophenyl)-2,8-diazaspiro[4.5]decan-8-yl]-7-[(2-methoxyethyl)(methyl)amino]-1-methyl-2-oxo-1,2-dihydroquinoline-3-carbonitrile;
7-[(2-methoxyethyl)(methyl)amino]-1-methyl-2-oxo-4-{2-[3-(trifluoromethoxy)phenyl]-2,8-diazaspiro[4.5]decan-8-yl}-1,2-dihydroquinoline-3-carboxamide; and
4-[2-(3-chlorophenyl)-2,8-diazaspiro[4.5]decan-8-yl]-7-[(2-methoxyethyl)(methyl)amino]-1-methyl-2-oxo-1,2-dihydroquinoline-3-carboxamide,
or a stereoisomer, a tautomer, an N-oxide, a hydrate, a solvate, or a salt thereof, or a mixture of any of the foregoing.

8: A method for treatment or prophylaxis of a disease, comprising administering a therapeutically effective amount of the compound of claim 1, or a pharmaceutically acceptable salt thereof, to a mammal in need thereof.

9: A pharmaceutical composition, comprising the compound of claim 1 and one or more pharmaceutically acceptable excipients.

10: A pharmaceutical combination comprising:
one or more compounds of claim 1, and
one or more further active ingredients.

11-14: (canceled)

15: The method of claim 8, wherein the disease is cancer, a condition with dysregulated immune response, or a disorder associated with aberrant DGKα signalling.

16: The pharmaceutical combination of claim 10, wherein the one or more further active ingredients comprises an immune checkpoint inhibitor.

17: The pharmaceutical combination of claim 16, wherein the immune checkpoint inhibitor is an aPD-1/L-1 axis antagonist.

18: The pharmaceutical combination of claim 16, wherein the immune checkpoint inhibitor is an inhibitor of DKGζ.


SEQUENCE LISTING (5 sequences)

SEQ ID NO: 1 - protein, 743 aa, Homo sapiens
  human DGKa M1 to S735 plus C-terminal Flag-Tag
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSDYKDDDDK

SEQ ID NO: 2 - protein, 793 aa, Homo sapiens
  human DGKa M1 to S735 plus N-terminal Avi-Tag and C-terminal Flag-Tag, DGKa_hu_1Avi
MTSHHHHHHSSMGSRGIEGRGGLNDIFEAQKIEWHEGSRTSLYKKAGSATMAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSDYKDDDDK

SEQ ID NO: 3 - protein, 8 aa, Homo sapiens
  SIINFEKL amino acid sequence
SIINFEKL

SEQ ID NO: 4 - protein, 8 aa, Homo sapiens
  Flag-Tag sequence
DYKDDDDK

SEQ ID NO: 5 - protein, 30 aa, Homo sapiens
  OVA-30 peptide sequence
SMLVLLPDEVSGLEQLESIINFEKLTEWTS